(19) United States
(12) Patent Application Publication
(10) Pub. No.: US 2003/0068788 A1
(43) Pub. Date: Apr. 10, 2003

(54) Methods and compositions for making emamectin

(76) Inventors: Thomas Gunther Buckel, Freiburg (DE); Philip Eugene Hammer, Cary, NC (US); Dwight Steven Hill, Cary, NC (US); James Madison Ligon, Apex, NC (US); Istvan Molnar Durham, Durham, NC (US); Johannes Paul Pachlatko, Seltisberg (CH); Ross Eric Zirkle, Raleigh, NC (US)

Correspondence Address: HALE AND DORR, LLP, 60 STATE STREET, BOSTON, MA, 02109

(21) Appl. No.: 10/145,415

(22) Filed: May 14, 2002

Related U.S. Application Data
(60) Provisional application No. 60/291,149, filed on May 16, 2001.

(51) Int. Cl. C12P 19/62; C12P 21/02; C12N 1/21; C12N 9/04; C12N 9/06; C12N 15/74

(52) U.S. Cl. 435/76; 514/29; 435/69.1; 435/190; 435/252.3; 435/320.1; 536/23.2; 435/191

(57) ABSTRACT

Disclosed is a family of P450 monooxygenases, each member of which regioselectively oxidizes avermectin to 4″-keto-avermectin. The P450 monooxgenases find use in methods and formulations for making emamectin from avermectin. Also disclosed are methods for purifying the P450 monooxygenases of the invention, binding agents that specifically bind to the P450 monooxygenases of the invention, and genetically engineered cells that express the P450 monooxygenases of the invention. Also disclosed are ferredoxins and ferredoxin reductases that are active with the P450 monooxygenases of the invention.

### CROSS-REFERENCE TO RELATED APPLICATIONS

This application claims priority to U.S. Provisional Patent Application Serial No. 60/291,149 filed May 16, 2001, the entire contents of which are hereby incorporated by reference.

### BACKGROUND OF THE INVENTION

1. Field of the Invention

The invention relates to the field of agrochemicals, and in particular, to insecticides. More specifically, this invention relates to the derivatization of avermectin, particularly for the synthesis of emamectin.

1. Summary of the Related Art

Emamectin is a potent insecticide and controls many pests such as thrips, leafminers, and worm pests including alfalfa caterpillar, beet armyworm, cabbage looper, corn earworm, cutworms, diamondback moth, tobacco budworm, tomato fruitworm, and tomato pinworm. Emamectin (4″-deoxy-4″-epi-N-methylamino avermectin B1a/B1b) is described in U.S. Pat. No. 4,874,749 and in Cvetovich, R. J. et al, J. Organic Chem. 59:7704-7708, 1994 (as MK-244).

U.S. Pat. No. 5,288,710 describes salts of emamectin that are especially valuable agrochemically. These salts of emamectin are valuable pesticides, especially for combating insects and representatives of the order Acarina. Some pests for which emamectin is useful in combating are listed in European Patent Application EP-A 736,252.

One drawback to the use of emamectin is the difficulty of its synthesis from avermectin. This is due to the first step of the process, which is the most costly and time-consuming step of producing emamectin, in which the 4″-carbinol group of avermectin must be oxidized to a ketone. The oxidation of the 4″-carbinol group is problematic due to the presence of two other hydroxyl groups on the molecule that must be chemically protected before oxidation and deprotected after oxidation. Thus, this first step, significantly increases the overall cost and time of producing emamectin from avermectin.

Because of the efficacy and potency of emamectin as an insecticide, there is a need to develop a cost and time effective method and/or reagent for regioselectively oxidizing the 4″-carbinol group of avermectin to produce 4″-keto-avermectin, which is a necessary intermediate for producing emamectin from avermectin.

### BRIEF SUMMARY OF THE INVENTION

The invention provides a novel family of P450 monooxygenases, each member of which is able to regioselectively oxidize the 4″-carbinol group of unprotected avermectin, thereby resulting in a cheap, effective method to produce 4″-keto-avermectin, a necessary intermediate in the production of emamectin. The invention allows elimination of the costly, time-consuming steps of (1) chemically protecting the two other hydroxyl groups on the avermectin molecule prior to oxidation of the 4″-carbinol group that must be chemically protected before oxidation; and (2) chemically deprotecting these two other hydroxyl groups after oxidation. The invention thus provides reagents and methods for significantly reducing the overall cost of producing emamectin from avermectin.

Accordingly, in one aspect, the invention provides a purified nucleic acid molecule encoding a polypeptide that exhibits an enzymatic activity of a P450 monooxygenase and is capable of regioselectively oxidizing the alcohol at position 4″ of a compound of formula (II), in free form or in salt form

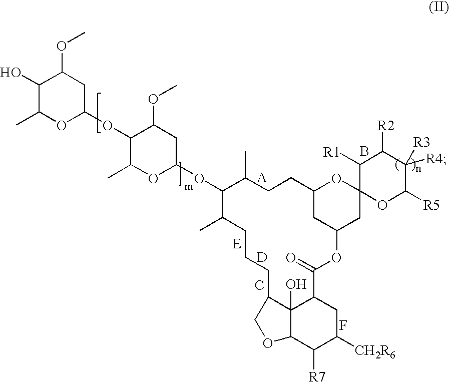

wherein R₁-R₇ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula

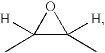

or a single bond and a methylene bridge of the formula

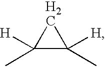

including, where applicable, an E/Z isomer, a mixture of E/Z isomers, and/or a tautomer thereof, in order to produce a compound of the formula (III), in free form or in salt form

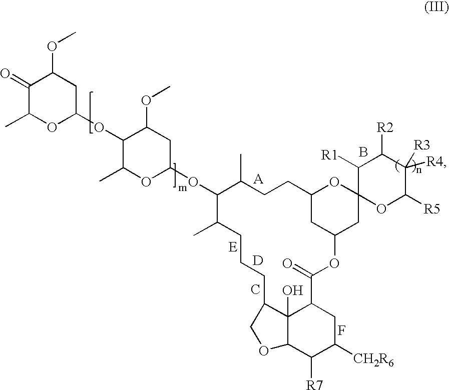

in which R1, R2, R3, R4, R5, R6, R7, m, n, A, B, C, D, E and F have the meanings given for formula (II).

In another aspect, the invention provides a purified nucleic acid molecule encoding a P450 monooxygenase that regioselectively oxidizes avermectin to 4″-keto-avermectin. In certain embodiments, the nucleic acid molecule comprises or consists essentially of a nucleic acid sequence that is at least 66% identical to SEQ ID NO: 1. In certain embodiments, the nucleic acid molecule comprises or consists essentially of a nucleic acid sequence that is at least 70% identical to SEQ ID NO: 1, SEQ ID NO: 3, SEQ ID NO: 5, SEQ ID NO: 7, SEQ ID NO:9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31,SEQ ID NO: 33, or SEQ ID NO: 94. In some embodiments, the nucleic acid molecule comprises or consists essentially of a nucleic acid sequence that is at least 80% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO:23, SEQ ID NO: 25, SEQ ID NO: 27, SEQ ID NO: 29, SEQ ID NO: 31, SEQ ID NO: 33, or SEQ ID NO: 94. In certain embodiments, the nucleic acid molecule comprises or consists essentially of a nucleic acid sequence that is at least 90% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94. In certain embodiments, the nucleic acid molecule comprises or consists essentially of a nucleic acid sequence that is at least 95% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94.

In some embodiments, the nucleic acid molecule comprises or consists essentially of a nucleic acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94.

In particular embodiments, the nucleic acid molecule is isolated from a Streptomyces strain. In certain embodiments, the Streptomyces strain is selected from the group consisting of Streptomyces tubercidicus, Streptomyces lydicus, Streptomyces platensis, Streptomyces chattanoogensis, Streptomyces kasugaensis, Streptomyces rimosus, and Streptomyces albofaciens.

In some embodiments of this aspect, the nucleic acid molecule further comprises a nucleic acid sequence encoding a tag which is linked to the P450 monooxygenase via a covalent bond. In certain embodiments, the tag is selected from the group consisting of a His tag, a GST tag, an HA tag, a HSV tag, a Myc-tag, and VSV-G-Tag.

In another aspect, the invention provides a purified polypeptide that exhibits an enzymatic activity of a P450 monooxygenase and is capable of regioselectively oxidizing the alcohol at position 4″ of a compound of formula (II), in free form or in salt form

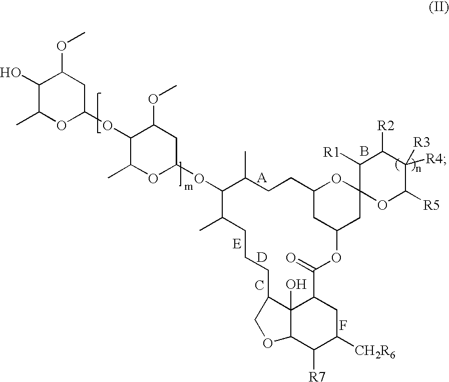

wherein R₁-R₇ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula

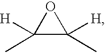

or a single bond and a methylene bridge of the formula

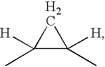

including, where applicable, an E/Z isomer, a mixture of E/Z isomers, and/or a tautomer thereof, in order to produce a compound of the formula (III), in free form or in salt form

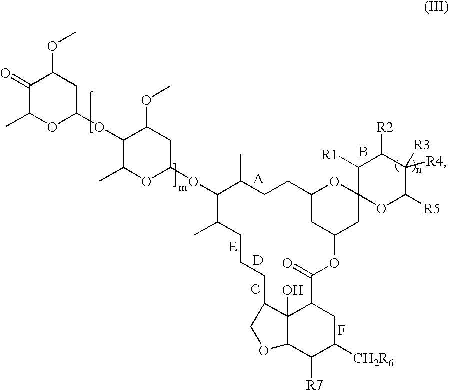

in which R1, R2, R3, R4, R5, R6, R7, m, n, A, B, C, D, E and F have the meanings given for formula (II).

In another aspect, the invention provides a purified P450 monooxygenase that regioselectively oxidizes avermectin to 4″-keto-avermectin. In some embodiments, the P450 monooxygenase comprises or consists essentially of an amino acid sequence that is at least 50% identical to SEQ ID NO: 2. In some embodiments, the P450 monooxygenase comprises or consists essentially of an amino acid sequence that is at least 60% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In certain embodiments, the P450 monooxygenase comprises or consists essentially of an amino acid sequence that is at least 70% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In some embodiments, the P450 monooxygenase comprises or consists essentially of an amino acid sequence that is at least 80% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In some embodiments, the P450 monooxygenase comprises or consists essentially of an amino acid sequence that is at least 90% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In certain embodiments, the P450 monooxygenase comprises or consists essentially of an amino acid sequence that is at least 95% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95.

In some embodiments of this aspect of the invention, the P450 monooxygenase comprises or consists essentially of an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, and SEQ ID NO: 95.

In certain embodiments, the P450 monooxygenase further comprises a tag. In some embodiments, the tag is selected from the group consisting of a His tag, a GST tag, an HA tag, a HSV tag, a Myc-tag, and VSV-G-Tag.

In another aspect, the invention provides a binding agent that specifically binds to a P450 monooxygenase that regioselectively oxidizes avermectin to 4″-keto-avermectin. In some embodiments, the binding agent is an antibody. In certain embodiments, the antibody is a polyclonal antibody or a monoclonal antibody.

In yet another aspect, the invention provides a family of P450 monooxygenase polypeptides, wherein each member of the family regioselectively oxidizes avermectin to 4″-keto-avermectin. In certain embodiments, each member of the family comprises or consists essentially of an amino acid sequence that is at least 50% identical to SEQ ID NO: 2. In certain embodiments, each member of the family comprises or consists essentially of an amino acid sequence that is at least 60% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In some embodiments, each member of the family comprises or consists essentially of an amino acid sequence that is at least 70% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In certain embodiments, each member of the family comprises or consists essentially of an amino acid sequence that is at least 80% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In some embodiments, each member of the family comprises or consists essentially of an amino acid sequence that is at least 90% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In certain embodiments, each member of the family comprises or consists essentially of an amino acid sequence that is at least 95% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In some embodiments of this aspect of the invention, each member of the family comprises or consists essentially of an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, and SEQ ID NO: 95.

In still another aspect, the invention provides a cell genetically engineered to comprise a nucleic acid molecule encoding a P450 monooxygenase that regioselectively oxidizes avermectin to 4″-keto-avermectin. In some embodiments, the nucleic acid molecule is positioned for expression in the cell. In certain embodiments, the cell further comprises a nucleic acid molecule encoding a ferredoxin protein. In some embodiments, the cell further comprises a nucleic acid molecule encoding a ferredoxin reductase protein.

In certain embodiments, the cell is a genetically engineered Streptomyces strain. In some embodiments, the cell is a genetically engineered Streptomyces lividans strain. In particular embodiments, the genetically engineered Streptomyces lividans strain has NRRL Designation No. B-30478. In particular embodiments, the cell is a genetically engineered Pseudomonas strain. In some embodiments, the cell is a genetically engineered Pseudomonas putida strain. In certain embodiments, the genetically engineered Pseudomonas putida strain has NRRL Designation No. B-30479. In some embodiments, the cell is a genetically engineered Escherichia coli strain.

In another aspect, the invention provides a purified nucleic acid molecule encoding a ferredoxin, wherein the nucleic acid molecule is isolated from a Streptomyces strain comprising a P450 monooxygenase that regioselectively oxidizes avermectin to 4″-keto-avermectin. In certain embodiments, the nucleic acid molecule encoding a ferredoxin of the invention comprises or consists essentially of a nucleic acid sequence that is at least 81% identical to SEQ ID NO: 35 or SEQ ID NO: 37. In some embodiments, the nucleic acid molecule comprises or consists essentially of a nucleic acid sequence that is at least 85%, or at least 90%, or at least 95%, or at least 99% identical to SEQ ID NO: 35 or SEQ ID NO: 37. In certain embodiments, the nucleic acid molecule encoding a ferredoxin of the invention comprises or consists essentially of the nucleic acid sequence of SEQ ID NO: 35 or SEQ ID NO: 37.

In yet another aspect, the invention provides a purified ferredoxin protein, wherein the ferredoxin protein is isolated from a Streptomyces strain comprising a P450 monooxygenase that regioselectively oxidizes avermectin to 4″-keto-avermectin. In certain embodiments, the ferredoxin of the invention comprises or consists essentially of an amino acid sequence that is at least 80% identical to SEQ ID NO: 36 or SEQ ID NO: 38. In some embodiments, the nucleic acid molecule comprises or consists essentially of an amino acid sequence that is at least 85%, or at least 90%, or at least 95%, or at least 99% identical to SEQ ID NO: 36 or SEQ ID NO: 38. In particular embodiments, the ferredoxin of the invention comprises or consists essentially of the amino acid sequence of SEQ ID NO: 36 or SEQ ID NO: 38.

In another aspect, the invention provides a purified nucleic acid molecule encoding a ferredoxin reductase, wherein the nucleic acid molecule is isolated from a Streptomyces strain comprising a P450 monooxygenase that regioselectively oxidizes avermectin to 4″-keto-avermectin. In certain embodiments, the nucleic acid molecule encoding a ferredoxin reductase of the invention comprises or consists essentially of the nucleic acid sequence of SEQ ID NO: 98, SEQ ID NO: 100, SEQ ID NO: 102, or SEQ ID NO: 104.

In yet another aspect, the invention provides a purified ferredoxin reductase protein, wherein the ferredoxin reductase protein is isolated from a Streptomyces strain comprising a P450 monooxygenase that regioselectively oxidizes avermectin to 4″-keto-avermectin. In certain embodiments, the ferredoxin reductase of the invention comprises or consists essentially of the amino acid sequence of SEQ ID NO: 99, SEQ ID NO: 101, SEQ ID NO: 103, or SEQ ID NO: 105.

In another aspect, the invention provides a process for the preparation a compound of the formula (I) in free form or in salt form

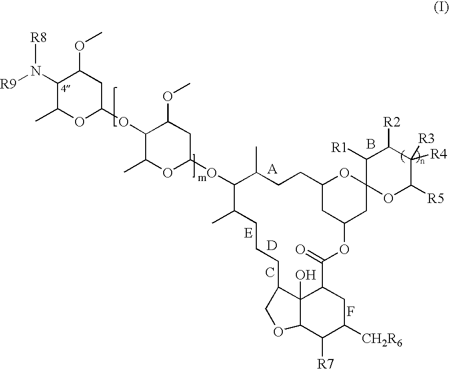

in which R₁-R₉ represent, independently of each other hydrogen or a substituent; m is 0, 1 or 2; n is 0, 1, 2, or 3; and the bonds marked with A, B, C, D, E, and F indicate, independently of each other, that two adjacent carbon atoms are connected by a double bond, a single bond, a single bond and a epoxide bridge of the formula

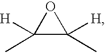

or a single bond and a methylene bridge of the formula

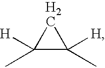

including, where applicable, an E/Z isomer, a mixture of E/Z isomers, and/or a tautomer thereof, which process comprises

1) bringing a compound of the formula (II), in free form or in salt form

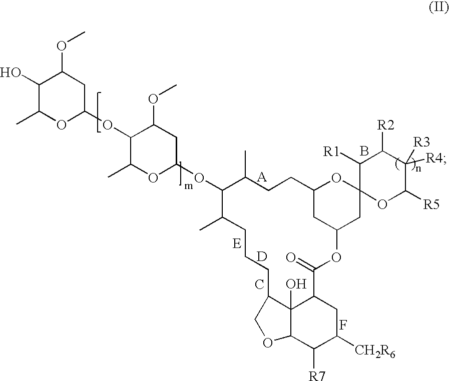

wherein R₁-R₇ represent, independently of each other hydrogen or a substituent; and m, n, A, B, C, D, E and F have the same meanings as given for formula (I) above, into contact with a polypeptide according to the invention that regioselectively oxidizes the alcohol at position 4″in order to form a compound of the formula (III), in free form or in salt form

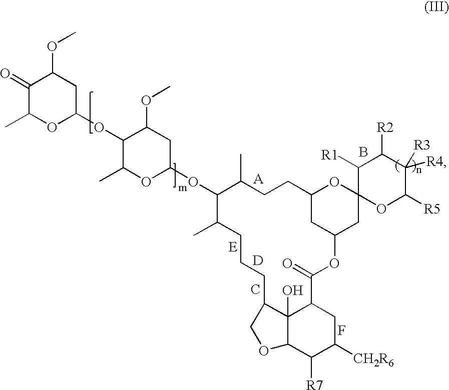

in which R₁-R₇ represent, independently of each other hydrogen or a substituent; and m, n, A, B, C, D, E and F have the meanings given for formula (I); and

2) reacting the compound of the formula (III) with an amine of the formula HN(R₈)R₉, wherein R₈ and R₉ have the same meanings as given for formula (I), and which is known, in the presence of a reducing agent; and, in each case, if desired, converting a compound of formula (I) obtainable in accordance with the process or by another method, or an E/Z isomer or tautomer thereof, in each case in free form or in salt form, into a different compound of formula (I) or an E/Z isomer or tautomer thereof, in each case in free form or in salt form, separating a mixture of E/Z isomers obtainable in accordance with the process and isolating the desired isomer, and/or converting a free compound of formula (I) obtainable in accordance with the process or by another method, or an E/Z isomer or tautomer thereof, into a salt or converting a salt, obtainable in accordance with the process or by another method, of a compound of formula (I) or of an E/Z isomer or tautomer thereof into the free compound of formula (I) or an E/Z isomer or tautomer thereof or into a different salt.

In some embodiments, the compound of formula (II) is further brought into contact with a polypeptide according to the invention exhibiting an enzymatic activity of a ferredoxin. In certain embodiments, the compound of formula (II) is further brought into contact with a polypeptide according to the invention exhibiting an enzymatic activity of a ferredoxin reductase. In some embodiments, the compound of formula (II) is further brought into contact with a reducing agent (e.g., NADH or NADPH).

In still a further embodiment, the invention provides a process for the preparation of a compound of the formula (III), in free form or in salt form

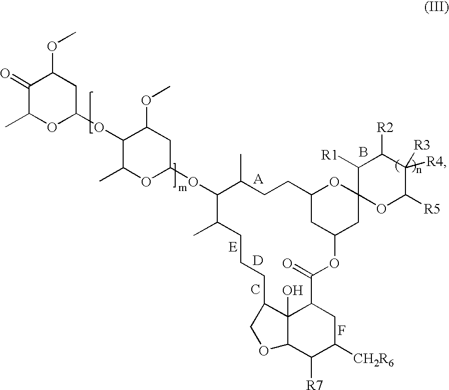

in which R₁-R₇ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula

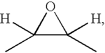

or a single bond and a methylene bridge of the formula

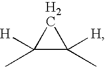

including, where applicable, an E/Z isomer, a mixture of E/Z isomers, and/or a tautomer thereof, which process comprises bringing a compound of the formula (II), in free form or in salt form

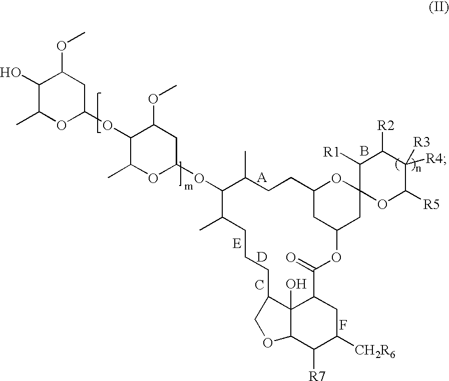

wherein R₁-R₇ represent, independently of each other hydrogen or a substituent; and m, n, A, B, C, D, E and F have the same meanings as given for formula (III) above, into contact with a polypeptide according to the invention that regioselectively oxidizes the alcohol at position 4″, and maintaining said contact for a time sufficient for the oxidation reaction to occur and isolating and purifying the compound of formula (III).

In yet another embodiment, the invention provides a process according to the invention for the preparation of a compound of the formula (I), in which n is 1; m is 1; A is a double bond; B is single bond or a double bond; C is a double bond; D is a single bond; E is a double bond; F is a double bond, or a single bond and a epoxy bridge, or a single bond and a methylene bridge; R₁, R₂ and R₃ are H; R₄ is methyl; R₅ is C₁-C₁₀ alkyl, C₃-C₈-cycloalkyl or C₂-C₁₀-alkenyl; R₆ is H; R₇ is OH; R₈ and R₉ are independently of each other H; C₁-C₁₀-alkyl or C₁-C₁₀-acyl, or together form —(CH₂)_{q}—, where q is 4, 5 or 6.

In still another embodiment, the invention provides a process according to the invention for the preparation of a compound of the formula (I), in which n is 1; m is 1; A, B, C, E and F are double bonds; D is a single bond; R₁, R₂, and R₃ are H; R₄ is methyl; R₅ is s-butyl or isopropyl; R₆ is H; R₇ is OH; R₈ is methyl; and R₉ is H.

In still another embodiment, the invention provides a process according to the invention for the preparation of 4″-deoxy-4″-N-methylamino avermectin B₁ₐ/B_{1b} benzoate salt.

In another aspect, the invention provides a method for making emamectin. The method comprises adding a P450 monooxygenase, that regioselectively oxidizes avermectin to 4″-keto-avermectin, to a reaction mixture comprising avermectin and incubating the reaction mixture under conditions that allow the P450 monooxygenase to regioselectively oxidize avermectin to 4″-keto-avermectin. In some embodiments, the reaction mixture further comprises a ferredoxin. In certain embodiments, the reaction mixture further comprises a ferredoxin reductase. In some embodiments, the reaction mixture further comprises a reducing agent (e.g., NADH or NADPH).

In still another aspect, the invention provides a formulation for making a compound of formula (I) comprising a polypeptide according to the invention exhibiting a P450 monooxygenase activity that is capable of regioselectively oxidising the alcohol at position 4″ in order to form a compound of formula (II). In some embodiments, the formulation further comprises a polypeptide according to the invention exhibiting an enzymatic activity of a ferredoxin (e.g., a ferredoxin from cell or strain from which the P450 monooxygenase was isolated or derived).

In still another aspect, the invention provides a formulation for making emamectin comprising a P450 monooxygenase that regioselectively oxidizes avermectin to 4″-keto-avermectin. In some embodiments, the formulation further comprises a ferredoxin (e.g., a ferredoxin from cell or strain from which the P450 monooxygenase was isolated or derived). In certain embodiments, the formulation further comprises a ferredoxin reductase (e.g., a ferredoxin reductase from cell or strain from which the P450 monooxygenase was isolated or derived). In some embodiments, the formulation further comprises a reducing agent (e.g., NADH or NADPH). dr

### BRIEF DESCRIPTION OF THE DRAWINGS

FIGS. 1A and 1B are schematic representations of an HPLC chromatogram (FIG. 1A) and data (FIG. 1B) showing the conversion of avermectin B1a to 4″-hydroxy-avermectin B1a and 4″-keto-avermectin B1a (also called 4″-oxo-avermectin B1a) and a side product from the biocatalysis reaction by a non-limiting P450 monooxygenase of the invention, P450_{Ema1}. The HPLC chromatogram using HPLC protocol I to resolve the products is shown in FIG. 1A, and the peaks are identified in FIG. 1B by their retention times. The Y-axis of FIG. 1A shows the milli-absorbance units (mAU) at 243 nm.

FIG. 2 is a representation of an HPLC chromatogram showing the increased biocatalysis activity (ie., the ability to regioselectively oxidize avermectin to 4″-keto-avermectin) by Streptomyces tubercidicus R-922 UV Mutant as compared to wild-type Streptomyces tubercidicus R-922. The Y-axis shows the milli-absorbance units (mAU) at 243 nm.

FIG. 3 is a schematic representation of the alignment of the deduced amino acid sequences of P450 gene-specific PCR fragments derived from Streptomyces tubercidicus strain R-922 and the P450 monooxygenase from S. thermotolerans that is involved in the synthesis of carbomycin (Stol-ORFA) (GenBank Accession No. D30759) by the program Pretty from the University of Wisconsin Package version 10.1 (Altschul et al., Nucl. Acids Res. 25:3389-3402). Only the portion of Stol-ORFA that is homologous to the PCR fragments is shown. The O₂ and heme binding sites at each end are indicated. The consensus sequence is shown on the bottom line and includes only those residues that are completely conserved in all sequences compared.

FIG. 4 is a schematic representation of the alignment of the deduced amino acid sequences of P450 gene-specific PCR fragments derived from Streptomyces strain I-1529 and the P450 monooxygenase from S. thermotolerans that is involved in the synthesis of carbomycin (Stol-ORFA) (GenBank Accession No. D30759) by Pretty. Only the portion of Stol-ORFA that is homologous to the PCR fragments is shown. The O₂ and heme binding sites at each end are indicated. The consensus sequence is shown on the bottom line and includes only those residues that are completely conserved in all sequences compared.

FIG. 5 is a schematic representation of the alignment of the deduced amino acid sequence of the 600 bp P450 gene fragment from Example VIII with the amino acid sequences of peptide fragments derived from purified P450_{Ema1} enzyme from Example VII.

FIG. 6 is a schematic representation of the alignment of the deduced amino acid sequence of two non-limiting P450 monooxygenases of the invention, namely from Streptomyces strains R-922 and I-1529, that are involved in emamectin biosynthesis. These are compared to the amino acid sequence of a P450 monooxygenase from S. thermotolerans that is involved in the synthesis of carbomycin (Carb-P450) (GenBank Accession No. D30759). Conserved residues in all three P450's are shown on the bottom line of the figure as the “consensus” sequence.

FIG. 7 is a diagrammatic representation showing a map of plasmid pTBBKA. Recognition sites by the indicated restriction endonucleases are shown, along with the location of the site in the nucleotide sequence of the plasmid. Also shown are genes (e.g., kanamycin resistance “KanR”), and other functional aspects (e.g., Tip promoter) contained in the plasmid.

FIG. 8 is a diagrammatic representation showing a map of plasmid pTUA1A. Recognition sites by the indicated restriction endonucleases are shown, along with the location of the site in the nucleotide sequence of the plasmid. Also shown are genes (e.g., ampicillin resistance “AmpR”) and other functional aspects (e.g., Tip promoter) contained in the plasmid.

FIG. 9 is a representation of an HPLC chromatogram showing the oxidation of avermectin to 4″-keto-avermectin by S. lividans transformed with the pTBBKA-ema1, following induction of ema1 expression with 0, 0,5, or 5.0 μg/ml thiostrepton. The Y-axis shows the milli-absorbance units (mAU) at 243 nm.

FIG. 10 is a diagrammatic representation of a phylogenetic tree showing the relationships between the seventeen ema genes described herein based on the deduced amino acid sequences of their protein products.

FIG. 11 is a diagrammatic representation showing a map of plasmid pRK-ema1/fd233. This plasmid was derived by ligating a Bg1II fragment containing the ema1 and fd233 genes organized on a single transcriptional unit into the Bg1II site of the broad host-range plasmid pRK290. The ema1/fd233 genes are expressed by the tac promoter (Ptac), and they are followed by the tac terminator (Ttac). Restriction endonuclease recognition sites shown are Bg1II “B”; EcoRI “E”; PacI “Pc”; PmeI “Pm”; and Sa1I “S.”

### DETAILED DESCRIPTION OF THE PREFERRED EMBODIMENTS

The patent and scientific literature referred to herein establishes knowledge that is available to those with skill in the art. The issued patents, applications, and references, including GenBank database sequences, that are cited herein are hereby incorporated by reference to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

More particularly, the family of polypeptides according to the invention may be used in a process for the preparation a compound of the formula (I), in free form or in salt form

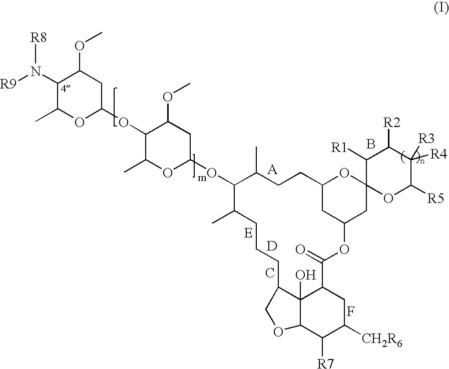

in which R₁-R₉ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula

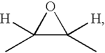

or a single bond and a methylene bridge of the formula

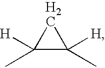

including, where applicable, an E/Z isomer, a mixture of E/Z isomers, and/or a tautomer thereof, which process comprises

1) bringing a compound of the formula (II), in free form or in salt form

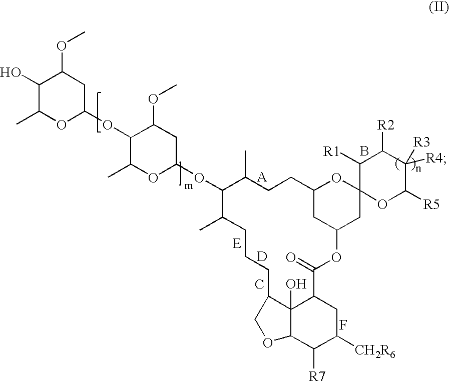

wherein R₁-R₇ represent, independently of each other hydrogen or a substituent; and m, n, A, B, C, D, E and F have the same meanings as given for formula (I) above, into contact with a polypeptide according to the invention which exhibits an enzymatic activity of a P450 monooxygenases and is capable of regioselectively oxidizing the alcohol at position 4″ of formula (II) in order to produce a compound of the formula (III), in free form or in salt form

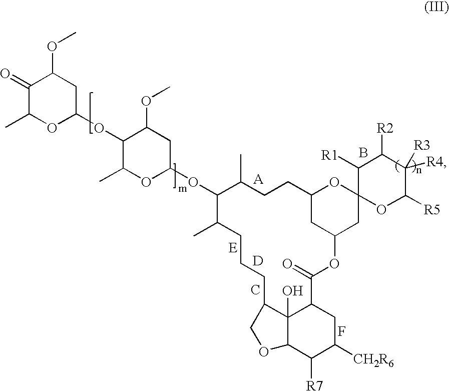

in which R₁-R₇ represent, independently of each other hydrogen or a substituent; and m, n, A, B, C, D, E and F have the meanings given for formula (I); and

2) reacting the compound of the formula (III) with an amine of the formula HN(R₈)R₉, wherein R₈ and R₉ have the same meanings as given for formula (I), and which is known, in the presence of a reducing agent; and, in each case, if desired, converting a compound of formula (I) obtainable in accordance with the process or by another method, or an E/Z isomer or tautomer thereof, in each case in free form or in salt form, into a different compound of formula (I) or an E/Z isomer or tautomer thereof, in each case in free form or in salt form, separating a mixture of E/Z isomers obtainable in accordance with the process and isolating the desired isomer, and/or converting a free compound of formula (I) obtainable in accordance with the process or by another method, or an E/Z isomer or tautomer thereof, into a salt or converting a salt, obtainable in accordance with the process or by another method, of a compound of formula (I) or of an E/Z isomer or tautomer thereof into the free compound of formula (I) or an E/Z isomer or tautomer thereof or into a different salt.

Methods of synthesis for the compounds of formula (I) are described in the literature. It has been found, however, that the processes known in the literature cause considerable problems during production basically on account of the low yields and the tedious procedures which have to be used. Accordingly, the known processes are not satisfactory in that respect, giving rise to the need to make available improved preparation processes for those compounds.

The compounds (I), (II) and (III) may be in the form of tautomers. Accordingly, hereinbefore and hereinafter, where appropriate the compounds (I), (II) and (III) are to be understood to include corresponding tautomers, even if the latter are not specifically mentioned in each case.

The compounds (I), (II), and (III) are capable of forming acid addition salts. Those salts are formed, for example, with strong inorganic acids, such as mineral acids, for example perchloric acid, sulfuric acid, nitric acid, nitrous acid, a phosphoric acid or a hydrohalic acid, with strong organic carboxylic acids, such as unsubstituted or substituted, for example halo-substituted, C₁-C₄alkanecarboxylic acids, for example acetic acid, saturated or unsaturated dicarboxylic acids, for example oxalic, malonic, succinic, maleic, fumaric or phthalic acid, hydroxycarboxylic acids, for example ascorbic, lactic, malic, tartaric or citric acid, or benzoic acid, or with organic sulfonic acids, such as unsubstituted or substituted, for example halo-substituted, C₁-C₄alkane- or aryl-sulfonic acids, for example methane- or p-toluene-sulfonic acid. Furthermore, compounds of formula (I), (II), and (III) having at least one acidic group are capable of forming salts with bases. Suitable salts with bases are, for example, metal salts, such as alkali metal or alkaline earth metal salts, for example sodium, potassium or magnesium salts, or salts with ammonia or an organic amine, such as morpholine, piperidine, pyrrolidine, a mono-, di- or tri-lower alkylamine, for example ethyl-, diethyl-, triethyl- or dimethyl-propyl-amine, or a mono-, di- or tri-hydroxy-lower alkylamine, for example mono-, di- or tri-ethanolamine. In addition, corresponding internal salts may also be formed. Particularly useful, within the scope of the invention, are agrochemically advantageous salts. In view of the close relationship between the compounds of formula (I), (II) and (III) in free form and in the form of their salts, any reference hereinbefore or hereinafter to the free compounds of formula (I), (II) and (III) or to their respective salts is to be understood as including also the corresponding salts or the free compounds of formula (I), (II) and (III), where appropriate and expedient. The same applies in the case of tautomers of compounds of formula (I), (II) and (III) and the salts thereof. The free form is generally useful in each case.

Useful, within the scope of this invention, is a process for the preparation of compounds of the formula (I), in which n is 1; m is 1; A is a double bond; B is single bond or a double bond; C is a double bond; D is a single bond; E is a double bond; F is a double bond, or a single bond and a epoxy bridge, or a single bond and a methylene bridge; R₁, R₂ and R₃ are H; R₄ is methyl; R₅ is C₁-C₁₀-alkyl, C₃-C₈-cycloalkyl or C₂-C₁₀-alkenyl; R₆ is H; R₇ is OH; R₈ and R₉ are independently of each other H; C₁-C₁₀-alkyl or C₁-C₁₀-acyl, or together form —(CH₂)_{q}—; and q is 4, 5 or 6.

Especially useful within the scope of this invention is a process for the preparation of a compound of the formula (I) in which n is 1; m is 1; A, B, C, E and F are double bonds; D is a single bond; R₁, R₂, and R₃ are H; R₄ is methyl; R₅ is s-butyl or isopropyl; R₆ is H; R₇ is OH; R8 is methyl; and R₉ is H.

Very especially useful is a process for the preparation of emamectin, more particularly the benzoate salt of emamectin. Emamectin is a mixture of 4″-deoxy-4″-N-methylamino avermectin B₁ₐ/B_{1b} and is described in U.S. Pat. No. 4,4874,749 and as MK-244 in J. Organic Chem. 59:7704-7708, 1994. Salts of emamectin that are especially valuable agrochemically are described in U.S. Pat. No. 5,288,710. Each member of this family of peptides exhibiting an enzymatic activity of a P450 monooxygenases as described hereinbefore is able to oxidize unprotected avermectin regioselectively at position 4″, thus opening a new and more economical route for the production of emamectin.

The family members each catalyze the following reaction:

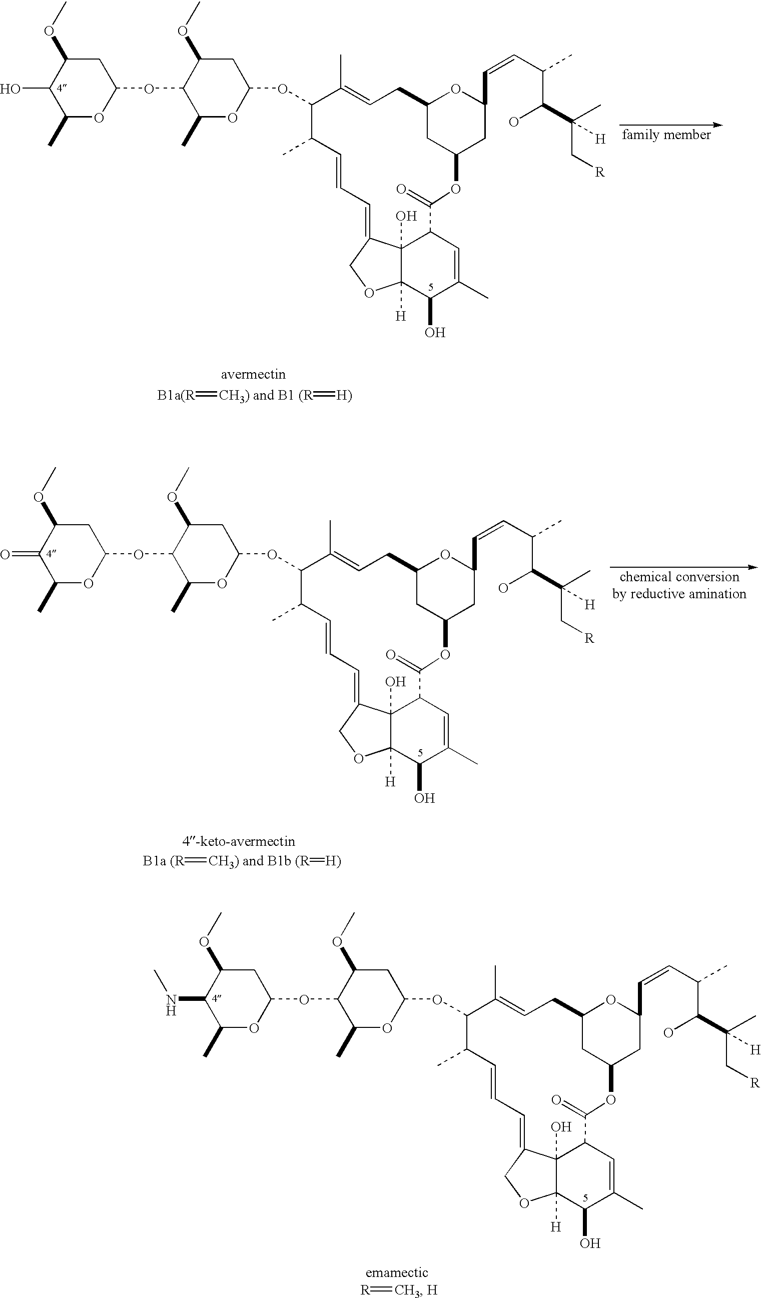

Accordingly, the invention provides a purified nucleic acid molecule encoding a polypeptide that exhibits an enzymatic activity of a P450 monooxygenase and is capable of regioselectively oxidizing the alcohol at position 4″ of a compound of formula (II) such as avermectin in order to produce a compound of formula (III), but especially 4″-keto-avermectin.

In particular, the invention provides a purified nucleic acid molecule encoding a P450 monooxygenase that regioselectively oxidizes avermectin to 4″-keto-avermectin. A “nucleic acid molecule” refers to single-stranded or double-stranded polynucleotides, such as deoxyribonucleic acid (DNA), ribonucleic acid (RNA), or analogs of either DNA or RNA.

The invention also provides a purified P450 monooxygenase that regioselectively oxidizes avermectin to 4″-keto-avermectin. As used herein, by “purified” is meant a nucleic acid molecule or polypeptide (e.g., an enzyme or antibody) that has been separated from components which naturally accompany it. For example, in the case of a nucleic acid molecule, the purified nucleic acid molecule is separated from nucleotide sequences, such as promoter or enhancer sequences, that flank the nucleic acid molecule as it naturally occurs in the chromosome. In the case of a protein, the purified protein is separated from components, such as other proteins or fragments of cell membrane, that accompany it in the cell. Of course, those of ordinary skill in molecular biology will understand that water, buffers, and other small molecules may additionally be present in a purified nucleic acid molecule or purified protein preparation. A purified nucleic acid molecule or purified polypeptide (e.g., enzyme) of the invention that is at least 95% by weight, or at least 98% by weight, or at least 99% by weight, or 100% by weight free of components which naturally accompany the nucleic acid molecule or polypeptide.

According to the invention, a purified nucleic acid molecule may be generated, for example, by excising the nucleic acid molecule from the chromosome. It may then be ligated into an expression plasmid. Other methods for generating a purified nucleic acid molecule encoding a P450 monooxygenase of the invention are available and include, without limitation, artificial synthesis of the nucleic acid molecule on a nucleic acid synthesizer.

Similarly, a purified P450 monooxygenase of the invention may be generated, for example, by recombinant expression of a nucleic acid molecule encoding the P450 monooxygenase in a cell in which the P450 monooxygenase does not naturally occur. Of course, other methods for obtaining a purified P450 monooxygenase of the invention include, without limitation, artificial synthesis of the P450 monooxygenase on a peptide synthesizer and isolation of the P450 monooxygenase from a cell in which it naturally occurs using, e.g., an antibody that specifically binds the P450 monooxygenase.

Biotransformations of secondary alcohols to ketones by Streptomyces bacteria are known to be catalyzed by dehydrogenases or oxidases. However, prior to the present discovery of the cytochrome P450 monooxygenase from Streptomyces tubercidicus strain R-922 responsible for the regioselective oxidation of avermectin to 4″-keto-avermectin, no experimental data of another cytochrome P450 monooxygenase from Streptomyces to oxidize a secondary alcohol to a ketone had been reported.

According to some embodiments of the invention, the nucleic acid molecule and/or the polypeptide encoded by the nucleic acid molecule are isolated from a Streptomyces strain. Thus, the nucleic acid molecule (or polypeptide encoded thereby) may be isolated from, without limitation, Streptomyces tubercidicus, Streptomyces lydicus, Streptomyces platensis, Streptomyces chattanoogensis, Streptomyces kasugaensis, Streptomyces rimosus, or Streptomyces albofaciens.

As described below, an entire family of P450 monooxygenases capable of regioselectively oxidizing avermectin to 4″-keto-avermectin has been discovered. All of these family members are related by at least 60% identity at the amino acid level. A useful nucleic acid molecule encoding a P450 monooxygenase of the invention comprises or consists essentially of a nucleic acid sequence that is at least 70% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94. In certain embodiments, the nucleic acid molecule encoding a P450 monooxygenase of the invention comprises or consists essentially of a nucleic acid sequence that is at least 80% identical; or at least 85% identical; or at least 90% identical; or at least 95% identical; or at least 98% identical to SEQ ID NO: 1, SEQ ID NO: 3, SEQ ID NO: 5, SEQ ID NO: 7, SEQ ID NO: 9, SEQ ID NO:I 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94.

Similarly, the invention provides a purified P450 monooxygenase that regioselectively oxidizes avermectin to 4″-keto-avermectin which, in some embodiments, comprises or consists essentially of an amino acid sequence that is at least 60% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In certain embodiments, the purified P450 monooxygenase of the invention comprises or consists essentially of an amino acid sequence that is at least 70% identical; or at least 80% identical; or at least 90% identical; or at least 95% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95.

In some embodiments, the nucleic acid molecule encoding a P450 monooxygenase of the invention comprises or consists essentially of the nucleic acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94. Similarly, the P450 monooxygenase of the invention, in some embodiments, comprises or consists essentially of the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95.

One non-limiting source of a purified P450 monooxygenase that regioselectively oxidizes avermectin to 4″-keto-avermectin is the cell-free extract described in the examples below. Another method for purifying a P450 monooxygenase in accordance with the invention is to attach a tag to the protein, thereby facilitating its purification. Accordingly, the invention provides a P450 monooxygenase which regioselectively oxidizes avermectin to 4″-keto-avermectin, wherein the P450 monooxygenase is covalently bound to a tag. The invention further provides a nucleic acid molecule encoding such a tagged P450 monooxygenase.

As used herein, a “tag” is meant a peptide or other molecule covalently bound to a polypeptide of the invention, whereby a binding agent (e.g., a polypeptide or molecule) specifically binds the tag. In accordance with the invention, by “specifically binds” is meant that the binding agent (e.g., an antibody or Ni²⁺resin) recognizes and binds to a particular polypeptide or chemical but does not substantially recognize or bind to other molecules in the sample. In some embodiments, a binding agent that specifically binds a ligand forms an association with that ligand with an affinity of at least 10⁶M⁻¹, or at least 10⁷M⁻¹, or at least 10⁸M⁻¹, or at least 10⁹M⁻¹ either in water, under physiological conditions, or under conditions which approximate physiological conditions with respect to ionic strength, e.g., 140 mM NaCl, 5 mM MgCl₂. For example, a His tag is specifically bound by nickel (e.g., the Ni²⁺-charged column commercially available as His•Bind® Resin from Novagen Inc, Madison, Wis.). Likewise, a Myc tag is specifically bound by an antibody that specifically binds Myc.

As described below, a his tag is attached to the P450 monooxygenases of the invention by generating a nucleic acid molecule encoding the His-tagged polypeptide, and expressing the polypeptide in E. coli. These polypeptides, once expressed by E. coli, are readily purified by standard techniques (e.g., using one of the His•Bind® Kits commercially available from Novagen or using the TALON™ Resin (and manufacturer's instructions) commercially available from Clontech Laboratories, Inc., Palo Alto, Calif.).

Additional tags may be attached to any or all of the P450 monooxygenases of the invention to facilitate purification. These tags include, without limitation, the HA-Tag (amino acid sequence: YPYDVPDYA (SEQ ID NO: 39)), the Myc-tag (amino acid sequence: EQKLISEEDL (SEQ ID NO: 40)), the HSV tag (amino acid sequence: QPELAPEDPED (SEQ ID NO: 41)), and the VSV-G-Tag (amino acid sequence: YTDIEMNRLGK (SEQ ID NO: 42)). Covalent attachment (e.g., via a peptide bond) of these tags to a polypeptide of the invention allows purification of the tagged polypeptide using, respectively, an anti-HA antibody, an anti-Myc antibody, an anti-HSV antibody, or an anti-VSV-G antibody, all of which are commercially available (for example, from MBL International Corp., Watertown, Mass.; Novagen Inc.; Research Diagnostics Inc., Flanders, N.J.).

The tagged P450 monooxygenases of the invention may also be tagged by a covalent bond to a chemical, such as biotin, which is specifically bound by streptavidin, and thus may be purified on a streptavidin column. Similarly, the tagged P450 monooxygenases of the invention may be covalently bound (e.g., via a peptide bond) to the constant region of an antibody. Such a tagged P450 monooxygenase may be purified, for example, on protein A sepharose.

The tagged P450 monooxygenases of the invention may also be tagged to a GST (glutathione-S-transferase) or the constant region of an immunoglobulin. For example, a nucleic acid molecule of the invention (e.g., comprising SEQ ID NO: 1) can be cloned into one of the pGEX plasmids commercially available from Amersham Pharmacia Biotech, Inc. (Piscataway N.J.), and the plasmid expressed in E. coli. The resulting P450 monooxygenase encoded by the nucleic acid molecule is covalently bound to a GST (glutathione-S-transferase). These GST fusion proteins can be purified on a glutathione agarose column (commercially available from, e.g., Amersham Pharmacia Biotech), and thus purified. Many of the pGEX plasmids enable easy removal of the GST portion from the fusion protein. For example, the pGEX-2T plasmid contains a thrombin recognition site between the inserted nucleic acid molecule of interest and the GST-encoding nucleic acid sequence. Similarly, the pGES-3T plasmid contains a factor Xa site. By treating the fusion protein with the appropriate enzyme, and then separating the GST portion from the P450 monooxygenase of the invention using glutathione agarose (to which the GST specifically binds), the P450 monooxygenase of the invention can be purified.

Yet another method to obtain a purified P450 monooxygenase of the invention is to use a binding agent that specifically binds to the P450 monooxygenase. Accordingly, the invention provides a binding agent that specifically binds to a P450 monooxygenase of the invention. This binding agent of the invention may be a chemical compound (e.g., a protein), a metal ion, or a small molecule.

In particular embodiments, the binding agent is an antibody. The term “antibody” encompasses, without limitation, polyclonal antibody, monoclonal antibody, antibody fragments (e.g., Fab, Fv, or Fab′ fragments), single chain antibody, chimeric antibody, bi-specific antibody, antibody of any isotype (e.g., IgG, IgA, and IgE), and antibody from any specifies (e.g., rabbit, mouse, and human).

In one non-limiting example, the binding agent of the invention is a polyclonal antibody. In another non-limiting example, the binding agent of the invention is a monoclonal antibody. Methods for making both monoclonal and polyclonal antibodies are well known (see, e.g., Current Protocols in Immunology, ed. John E. Coligan, John Wiley & Sons, Inc. 1993; Current Protocols in Molecular Biology, eds. Ausubel et a., John Wiley & Sons, Inc. 2000).

The P450 monooxygenases described herein that regioselectively oxidize avermectin to 4″-keto-avermectin belong to a family of novel P450 monooxygenases. Accordingly, the invention also provides a family of P450 monooxygenase polypeptides, wherein each member of the family regioselectively oxidizes avermectin to 4″-keto-avermectin. In some embodiments, each member of the family comprises or consists of an amino acid sequence that is at least 50% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 95. In particular embodiments, each member of the family is encoded by a nucleic acid molecule comprising or consisting of a nucleic acid sequence that is at least 66% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 94.

The present invention, which provides an entire family of P450 monooxygenases, each member of which is able to regioselectively oxidize avermectin to 4″-keto-avermectin, allowed for the generation of an improved P450 monooxygenase, which may not be naturally occurring, but which regioselectively oxidizes avermectin to 4″-keto-avermectin with efficiency and with reduced undesirable side product. For instance, one of the members of the P450 monooxygenase family of the invention, P450_{Ema1} enzyme catalyzes a further oxidation that is not desirable, since the formation of 3″-O-demethyl-4″-keto-avermectin has been detected in the reaction by Streptomyces tubercidicus strain R-922 and by Streptomyces lividans containing the ema1 gene. The formation of 3″-O-demethyl-4″-keto-avermectin is brought about by the oxidation of the 3″-O-methyl group, whereby the hydrolytically labile 3″-O-hydroxymethyl group is formed which hydrolyzes to form formaldehyde and the 3″-hydroxyl group.

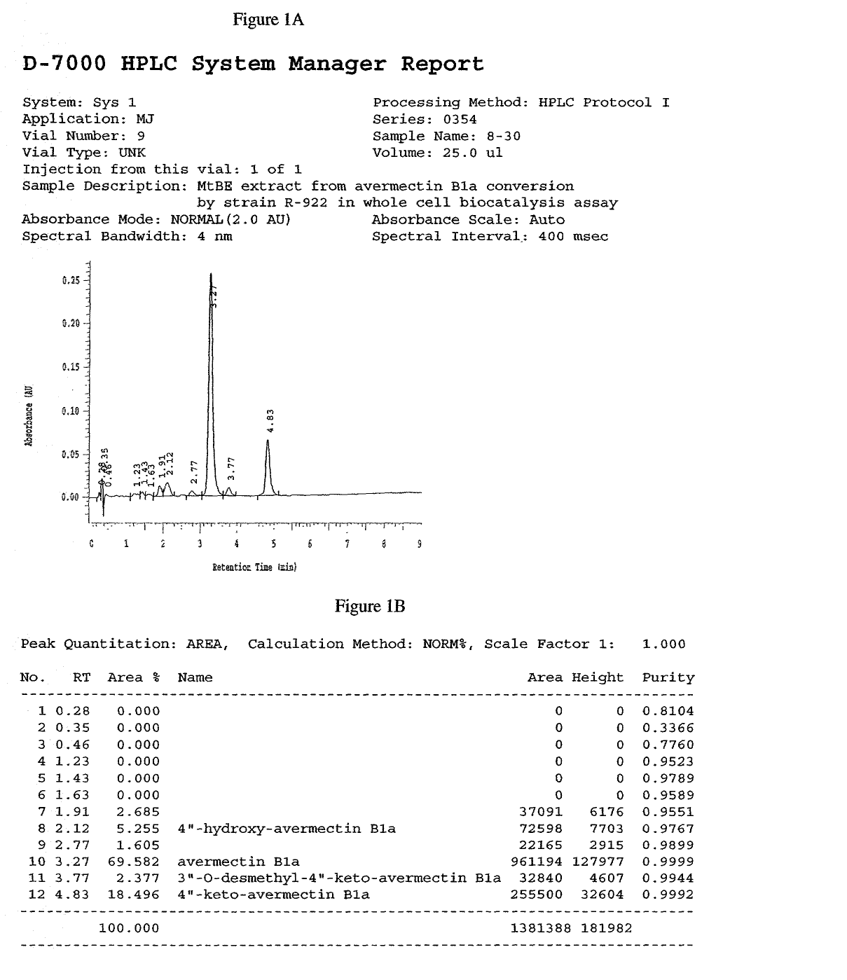

An HPLC chromatogram showing product and side product from the reaction is shown in FIGS. 1A and 1B.

By providing a family of P450 monooxygenases that regioselectively oxidize avermectin to 4″-keto-avermectin (see, e.g., Table 3 below), individual members of the family can be subjected to family gene shuffling efforts in order to produce new hybrid genes encoding optimized P450 monooxygenases of the invention. In one non-limiting example, a portion of the ema1 gene encoding the O₂ binding site of the P450_{Ema1} protein can be swapped with the portion of the ema2 gene encoding the O₂ binding site of the P450_{Ema2} protein. Such a chimeric ema1/2 protein is within definition of a P450 monooxygenase of the invention.

Site-directed mutagenesis or directed evolution technologies may also be employed to generate derivatives of the ema1 gene that encode enzymes with improved properties, including higher overall activity and/or reduced side product formation. One method for deriving such a mutant is to mutate the Streptomyces strain itself, in a manner similar to the UV mutation of Streptomyces tubercidicus strain R-922 described below.

Additional derivatives may be made by making conservative or non-conservative changes to the amino acid sequence of a P450 monooxygenase. Conservative and non-conservative amino acid substitutions are well known (see, e.g., Stryer, Biochemistry, 3^{rd} Ed., W. H. Freeman and Co., NY 1988). Similarly, truncations of a P450 monooxygenase of the invention may be generated by truncating the protein at its N-terminus (e.g., see the ema1A gene described below), at its C-terminus, or truncating (i.e., removing amino acid residues) from the middle of the protein.

Such a mutant, derivative, or truncated P450 monooxygenase is a P450 monooxygenase of the invention as long as the mutant, derivative, or truncated P450 monooxygenase is able to regioselectively oxidize avermectin to 4″-keto-avermectin.

In another aspect, the invention provides a cell genetically engineered to comprise a nucleic acid molecule encoding a P450 monooxygenase that regioselectively oxidizes avermectin to 4″-keto-avermectin. By “genetically engineered” is meant that the nucleic acid molecule is heterologous to the cell into which it is introduced. Introduction of the heterologous nucleic acid molecule into the genetically engineered cell may be accomplished by any means, including, without limitation, transfection, transduction, and transformation.

In certain embodiments, the nucleic acid molecule is positioned for expression in the genetically engineered cell. By “positioned for expression” is meant that the heterologous nucleic acid molecule encoding the polypeptide is linked to a regulatory sequence in such a way as to permit expression of the nucleic acid molecule when introduced into a cell. By “regulatory sequence” is meant nucleic acid sequences, such as initiation signals, polyadenylation (polyA) signals, promoters, and enhancers, which control expression of protein coding sequences with which they are operably linked. By “expression” of a nucleic acid molecule encoding a protein or polypeptide fragment is meant expression of that nucleic acid molecule as protein and/or mRNA.

A genetically engineered cell of the invention may be a prokaryotic cell (e.g., E. coli) or a eukaryotic cell (e.g., Saccharomyces cerevisiae or mammalian cell (e.g., HeLa)). According to some embodiments of the invention, the genetically engineered cell is a cell wherein the wild-type (i.e., not genetically engineered) cell does not naturally contain the inserted nucleic acid molecule and does not naturally express the protein encoded by the inserted nucleic acid molecule. Accordingly, the cell may be a genetically engineered Streptomyces strain, such as a Streptomyces lividans or a Streptomyces avermitilis strain. Alternatively, the cell may be a genetically engineered Pseudomonas strain, such as a Pseudomonas putida strain or a Pseudomonas fluorescens strain. In another alternative, the cell may be a genetically engineered Escherichia coli strain.

Note that in some types of cells genetically engineered to comprise a nucleic acid molecule encoding a P450 monooxygenase that regioselectively oxidizes avermectin to 4″-keto-avermectin, the actual genetically engineered cell, itself, may not be able to convert avermectin into 4″-keto-avermectin. Rather, the P450 monooxygenase heterologously expressed by such a genetically engineered cell may be purified from that cell, where the purified P450 monooxygenase of the invention is able to regioselectively oxidize avermectin to 4″-keto-avermectin. Thus, the genetically engineered cell of the invention need not, itself, be able to regioselectively convert avermectin to 4″-keto-avermection; rather, the genetically engineered cell of the invention need only comprise a nucleic acid molecule encoding a P450 monooxygenase that regioselectively oxidizes avermectin to 4″-keto-avermectin, regardless of whether the P450 monooxygenase is active inside that cell.

In addition, a cell (e.g., E. coli) geneticially engineered to comprise a nucleic acid molecule encoding P450 monooxygenase of the invention may not be able to regioselectively oxidize avermectin to 4″-keto-avermection, although the P450 monooxygenase purified from the genetically engineered cell is able to regioselectively oxidize avermectin to 4″-keto-avermectin. However, if the same cell were genetically engineered to comprise a P450 monooxygenase of the invention, a ferredoxin of the invention, and/or a ferredoxin reductase of the invention, then the P450 monooxygenase together with the ferredoxin and the ferredoxin reductase, all purified from that cell, and in the presence of a reducing agent (e.g., NADH or NADPH), would be able to regioselectively oxidize avermectin to 4″-keto-avermectin. Furthermore,-the genetically engineered cell comprising a P450 monooxygenase of the invention, a ferredoxin of the invention, and/or a ferredoxin reductase of the invention, itself would be able to carry out this oxidation.

Moreover, in a non-limiting example where a cell (e.g., E. coli) is genetically engineered to express P450 monooxygenase, a ferredoxin, and a ferredoxin reductase proteins of the invention, all three of these proteins, when purified from the genetically engineered E. coli, are active and together are able to regioselectively oxidize avermectin to 4″-keto-avermectin (e.g., in the presence of a reducing agent, such as NADH or NADPH), and so are useful in a method for making emamectin.

In accordance with the present invention, the following material has been deposited with the Agricultural Research Service, Patent Culture Collection (NRRL), 1815 North University Street, Peoria, Ill. 61604, under the terms of the Budapest Treaty on the International Recognition of the Deposit of Microorganisms for the Purposes of Patent Procedure: (1) Streptomyces lividans ZX7 (ema1/fd233-TUA1A) NRRL Designation No. B-30478; and (2) Pseudomonas putida NRRL B-4067 containing plasmid pRK290-ema1/fd233, NRRL Designation No.B-30479.

In identifying the novel family of P450 monooxygenases that regioselectively oxidize avermectin to 4″-keto-avermectin, novel ferredoxins and novel ferredoxin reductases were also discovered in the same strains of bacteria in which the P450 monooxygenases were found. Accordingly, in a further aspect, the invention provides purified nucleic acid molecule encoding a ferredoxin, wherein the nucleic acid molecule is isolated from a Streptomyces strain comprising a polypeptide that regioselectively oxidizes avermectin to 4″-keto-avermectin. Similarly, the invention provides a purified nucleic acid molecule encoding a ferredoxin reductase, wherein the nucleic acid molecule is isolated from a Streptomyces strain comprising a polypeptide that regioselectively oxidizes avermectin to 4″-keto-avermectin. The invention also provides a purified ferredoxin protein, as well as a purified ferredoxin reductase protein, wherein the ferredoxin protein and the ferredoxin reductase protein are isolated from a Streptomyces strain comprising a polypeptide that regioselectively oxidizes avermectin to 4″-keto-avermectin.

A useful nucleic acid molecule encoding a ferredoxin of the invention comprises or consists essentially of a nucleic acid sequence that is at least 81% identical to SEQ ID NO: 35 or SEQ ID NO: 37. Alternatively, the nucleic acid molecule comprises or consists essentially of a nucleic acid sequence that is at least 85%, or at least 90%, or at least 95%, or at least 99% identical to SEQ ID NO: 35 or SEQ ID NO: 37. The nucleic acid molecule encoding a ferredoxin of the invention may comprise or consist essentially of the nucleic acid sequence of SEQ ID NO: 35 or SEQ ID NO: 37.

The ferredoxin of the invention may comprise or consist essentially of an amino acid sequence that is at least 80% identical to SEQ ID NO: 36 or SEQ ID NO: 38. In some embodiments, the nucleic acid molecule comprises or consists essentially an amino acid sequence that is at least 85%, or at least 90%, or at least 95%, or at least 99% identical to SEQ ID NO: 36 or SEQ ID NO: 38. The ferredoxin of the invention may comprise or consist essentially of the amino acid sequence of SEQ ID NO: 36 or SEQ ID NO: 38.

A useful nucleic acid molecule encoding a ferredoxin reductase of the invention comprises or consists essentially of a nucleic acid sequence that is at least 85%, or at least 90%, or at least 95%, or at least 99% identical to SEQ ID NO: 98, SEQ ID NO: 100, SEQ ID NO: 102, or SEQ ID NO: 104. The nucleic acid molecule encoding a ferredoxin reductase of the invention may comprise or consist essentially of the nucleic acid sequence of SEQ ID NO: 98, SEQ ID NO: 100, SEQ ID NO: 102, or SEQ ID NO: 104. The ferredoxin reductase of the invention may comprise or consist essentially of an amino acid sequence that is at least 85%, or at least 90%, or at least 95%, or at least 99% identical to SEQ ID NO: 99, SEQ ID NO: 101, SEQ ID NO: 103,or SEQ ID NO: 105. The ferredoxin reductase of the invention may comprise or consist essentially of the amino acid sequence of SEQ ID NO: 99, SEQ ID NO: 101, SEQ ID NO: 103, or SEQ ID NO: 105.

Methods for purifying ferredoxin and ferredoxin reductase proteins and nucleic acid molecules encoding such ferredoxin and ferredoxin reductase proteins are known in the art and are the same as those described above for purifying P450 monooxygenases of the invention and nucleic acid molecules encoding P450 monooxygenases of the invention.

In one non-limiting example to obtain a purified P450 monooxygenase of the invention with a purified ferredoxin, a S. lividans strain (or P. putida strain, or any other cell in which the P450 monooxygenase of the invention does not naturally occur) may be genetically engineered to contain a first nucleic acid molecule encoding a P450 monooxygenase of the invention and a second nucleic acid molecule encoding a ferredoxin protein, where both the first and second nucleic acid molecules are positioned for expression in the genetically engineered cell. The first and the second nucleic acid molecules can be on separate plasmids, or can be on the same plasmid. Thus, the same engineered cell or strain will produce both the P450 monooxygenase of the invention and the ferredoxin protein of the invention.

In a further non-limiting example to obtain a purified P450 monooxygenase of the invention with a purified ferredoxin and with a purified ferredoxin reductase of the invention, a S. lividans strain (or P. putida strain, or any other cell in which the P450 monooxygenase of the invention does not naturally occur) may be genetically engineered to contain a first nucleic acid molecule encoding a P450 monooxygenase of the invention and a second nucleic acid molecule encoding a ferredoxin protein of the invention and a third nucleic acid molecule encoding a ferredoxin reductase protein of the invention, where all the first and second and third nucleic acid molecules are positioned for expression in the genetically engineered cell. The first and the second and the third nucleic acid molecules can be on separate plasmids, or can be on the same plasmid. Thus, the same engineered cell or strain will produce all the P450 monooxygenase of the invention and the ferredoxin protein and the ferredoxin reductase proteins of the invention.

As described above for the P450 monooxygenases of the invention, the ferredoxin protein and/or the ferredoxin reductase protein may further comprise a tag. Moreover, the invention contemplates binding agents (e.g., antibodies) that specifically bind to the ferredoxin protein, and binding agents that specifically bind to the ferredoxin reductase proteins of the invention. Methods for generating tagged ferredoxin protein, tagged ferredoxin reductase protein, and binding agents (e.g., antibodies) that specifically bind to ferredoxin or ferredoxin reductase are the same as those as described above for generating tagged P450 monooxygenases of the invention and generating binding agents that specifically bind P450 monooxygenases of the invention.

The invention also provides a method for making emamectin. In this method, a P450 monooxygenase that regioselectively oxidizes avermectin to 4″-keto-avermectin is added to a reaction mixture containing avermectin. The reaction mixture is then incubated under conditions that allow the P450 monooxygenase to regioselectively oxidize avermectin to 4″-keto-avermectin. The reaction mixture may further comprise a ferredoxin, such as a ferredoxin of the present invention. In particular embodiments, the reaction mixture further comprises a ferredoxin reductase, such as a ferredoxin reductase of the present invention. The reaction mixture may further comprise a reducing agent, such as NADH or NADPH.

Additionally, the invention provides a method for making 4″-keto-avermectin. The method comprises adding a P450 monooxygenase that regioselectively oxidizes avermectin to 4″-keto-avermectin to a reaction mixture comprising avermectin and incubating the reaction mixture under conditions that allow the P450 monooxygenase to regioselectively oxidize avermectin to 4″-keto-avermectin. In some embodiments, the reaction mixture further comprises a ferredoxin, such as a ferredoxin of the present invention. The reaction mixture may also further comprise a ferredoxin reductase, such as a ferredoxin reductase of the present invention. In particular embodiments, the reaction mixture further comprises a reducing agent, such as NADH or NADPH.

The invention also provides a formulation for making emamectin comprising a P450 monooxygenase that regioselectively oxidizes avermectin to 4″-keto-avermectin. In some embodiments, the formulation further comprises a ferredoxin, such as a ferredoxin of the present invention. In particular embodiments, the ferredoxin is isolated from the same species of cell or strain from which the P450 monooxygenase was isolated or derived. The formulation may further comprise a ferredoxin reductase, such as a ferredoxin reductase of the present invention. In particular embodiments, the ferredoxin reductase is isolated from the same species of cell or strain from which the P450 monooxygenase was isolated or derived. In some embodiments, the formulation further comprises a reducing agent, such as NADH or NADPH.

In addition, the invention provides a formulation for making 4″-keto-avermectin comprising a P450 monooxygenase that regioselectively oxidizes avermectin to 4″-keto-avermectin. In some embodiments, the formulation further comprises a ferredoxin, such as a ferredoxin of the present invention. In particular embodiments, the ferredoxin is isolated from the same species of cell or strain from which the P450 monooxygenase was isolated or derived. In some embodiments, the formulation further comprises a ferredoxin reductase, such as a ferredoxin reductase of the present invention. In particular embodiments, the ferredoxin reductase is isolated from the same species of cell or strain from which the P450 monooxygenase was isolated or derived. The formulation may further comprise a reducing agent, such as NADH or NADPH.

The following examples are intended to further illustrate certain preferred embodiments of the invention and are not limiting in nature.

### EXAMPLE I

### Optimized Growth Conditions for Streptomyces Tubercidicus Strain R-922

In one non-limiting example, the fermentation conditions needed to provide a steady supply of cells of Streptomyces tubercidicus strain R-922 highly capable of regioselectively oxidizing avermectin to 4″-keto-avermectin were optimized.

First, the following solutions were made. For ISP-2 agar, 4 g of yeast extract (commercially available from Oxoid Ltd, Basingstoke, UK), 4 g of D(+)-glucose, 10 g of bacto malt extract (Difco No. 0186-17-7 (Difco products commercially available from, e.g., Voigt Global Distribution, Kansas City, Mo.)), and 20 g of agar (Difco No. 0140-01) were dissolved in one liter of demineralized water, and the pH is adjusted to 7.0. The solution was sterilized at 121° C. for 20 min., cooled down, and kept at 55° C. for the time needed for the immediate preparation of the agar plates.

For PHG medium, 10 g of peptone (Sigma 0521; commercially available from Sigma Chemical Co., St. Louis, Mo.), 10 g of yeast extract (commercially available from Difco), 10 g of D-(+)-glucose, 2 g of NaCl, 0.15 g of MgSO₄×7 H₂O, 1.3 g of NaH₂PO₄×H₂O, and 4.4 g of K₂HPO₄ were dissolved in 1 liter of demineralized water, and the pH was adjusted to 7.0.

Streptomyces tubercidicus strain R-922 was grown in a Petri dish on ISP-2 agar at 28° C. This culture was used to inoculate four 500 ml shaker flasks with baffle, each containing 100 ml PHG medium. These pre-cultures were grown on an orbital shaker with 120 rpm at 28° C. for 72 hours and then used to inoculate a 10-liter fermenter equipped with a mechanical stirrer and containing 8 liters PHG medium. This main culture was grown at 28° C. with stirring at 500 rpm and with aeration of 1.75 vvm (14 l/min.) and a pressure of 0.7 bar. At the end of the exponential growth, after about 20 hours, the cells were harvested by centrifugation. The yield of wet cells was 70-80 g/l culture.

### EXAMPLE II

### Whole Cell Biocatalysis Assay

As determined in accordance with the present invention, the following whole cell biocatalysis assay was employed to determine that the activity from Streptomyces cells capable of regioselectively oxidizing avermectin to 4″-keto-avermectin is catalyzed by a P450 monooxygenase.

Streptomyces tubercidicus strain R-922 was grown in PHG medium, and Streptomyces tubercidicus strain I-1529 was grown in M-17 or PHG medium. PHG medium contains 10 g/l Peptone (Sigma, 0.521), 10 g/l Yeast Extract (Difco, 0127-17-9), 10 g/l D-Glucose, 2 g/l NaCl, 0.15 g/l MgSO₄×7 H₂O, 1.3 g/l NaH₂PO₄×1 H₂O, and 4.4 g/l K₂HPO₄ at pH 7.0. M-17 medium contains 10 g/l glycerol, 20 g/l Dextrin white, 10 g/l Soytone (Difco 0437-17), 3 g/l Yeast Extract (Difco 0127-17-9), 2 g/l (NH₄)₂SO₄, and 2 g/l CaCO₃ at pH 7.0

To grow the cells, an ISP2 agar plate (not older than 1-2 weeks) was inoculated and incubated for 3-7 days until good growth was achieved. Next, an overgrown agar piece was transferred (with an inoculation loop) to a 250 ml Erlenmeyer flask with 1 baffle containing 50 ml PHG medium. This pre-culture is incubated at 28° C. and 120 rpm for 2-3 days. Next, 5 ml of the pre-culture were transferred to a 500 ml Erlenmeyer flask with 1 baffle containing 100 ml PHG medium. The main culture was incubated at 28° C. and 120 rpm for 2 days. Next, the culture was centrifuged for 10 min. at 8000 rpm in a Beckman Rotor JA-14. The cells were next washed once with 50 mM potassium phosphate buffer, pH 7.0.

To perform the whole cell biocatalysis assay, 500 mg wet cells were placed into a 25 ml Erlenmeyer flask, to which were added 10 ml of 50 mM potassium phosphate buffer, pH 7.0. The cells were stirred with a magnetic stir bar to distribute the cells. Next, 15 μl of a solution of avermectin B1a in isopropanol (30 mg/ml) were added, and the mixture shaken on an orbital shaker at 160 rpm and 28° C. Strain R-922 was reacted for 2 hours, and strain I-1529 was reacted for 30 hours.

To work up the cultures in the whole cell biocatalysis assay, 10 ml methyl-t-butyl-ether was added to an Erlenmeyer flask containing the resting cells and the entire cell mixture was transferred to a 30 ml-centrifuge tube, shaken vigorously, and then centrifuged at 16000 rpm for 10 min. The ether phase was pipetted into a 50 ml pear flask, and evaporated in vacuo by means of a rotary evaporator (≦0.1 mbar). The residue was re-dissolved in 1.2 ml acetonitrile and transferred to an HPLC-sample vial. Formation of 4″-keto-avermectin B1a could be observed by HPLC analysis using HPLC protocol I.

For HPLC protocol I, the following parameters were used:

Hardware

| | | | |
|---|---|---|---|
| Pump: | L-6250 Merck-Hitachi | | |
| Autosampler: | AS-2000A Merck-Hitachi | | |
| Interface | D-6000 Merck-Hitachi | | |
| Module: | | | |
| Channel 1- | L-7450A UV-Diode Array | | |
| Detector: | Merck-Hitachi | | |
| Colunm Oven: | none | | |
| Column: | 70 mm × 4 mm | | |
| Adsorbent: | Kromasil 100 Å-3.5 μ-C18 | | |
| Gradient Mode: | Low | | |
| Pressure Limit: | 5-300 bar | | |
| Column | ambient (≈20° C.) | | |
| Temperature | | | |
| Solvent A: | acetonitrile | | |
| Solvent B: | water | | |
| Flow: | 1.5 ml/min | | |
| Detection: | 243 nm | | |
| Pump Table: | 0.0 min | 75% A | 25% B |
| linear gradient | 7.0 min | 100% A | 0% B |
| | 9.0 min | 100% A | 0% B |
| jump | 9.1 min | 75% A | 25% B |
| | 12.0 min | 75% A | 25% B |
| Stop time: | 12 min | | |
| Sampling | every 200 msec | | |
| Period: | | | |
| Retention time | time | References | |
| table: | 2.12 min | 4″-hydroxy- | |
| | | avermectin B1a | |
| | 3.27 min | avermectin B1a | |
| | 3.77 min | 3″-O-demethyl- | |
| | | 4″-keto- | |
| | | avermectin B1a | |
| | 4.83 min | 4″-keto- | |
| | | avermectin B1a | |

### EXAMPLE III

### Biotransformation With Cell-Free Extract From Streptomyces Strain R-922

To prepare an active cell-free extract from Streptomyces tubercidicus strain R-922 capable of regioselective oxidation of avermectin to 4″-keto-avermectin, the following solutions were made, stored at 4° C., and kept on ice when used.

| Solution | Formula |
|---|---|
| PP-buffer | 50 mM K₂HPO₄/KH₂PO₄ (pH 7.0) |
| Disruption buffer | 50 mM K₂HPO₄/KH₂PO₄ (pH 7.0), 5 mM benzamidine, 2 mM |
| | dithiothreitol, and 0.5 mM Pefabloc (from Roche |
| | Diagnostics) |
| Substrate | 10 mg avermectin were dissolved in 1 ml isopropanol |

Six grams of wet cells from Streptomyces strain R-922 were washed in PP-buffer and then resuspended in 35 ml disruption buffer and disrupted in a French press at 4° C. The resulting suspension was centrifuged for 1 hour at 35000×g. The supernatant of the cell free extract was collected. One μl substrate was added to 499 μl of cleared cell free extract and incubated at 30° C. for 1 hour. Then, 1 ml methyl-t-butyl ether was added to the reaction mixture and thoroughly mixed. The mixture was next centrifuged for 2 min. at 14000 rpm, and the methyl-t-butyl ether phase was transferred into a 10 ml flask and evaporated in vacuo by means of a rotary evaporator. The residue was dissolved in 200 μl acetonitrile and transferred into an HPLC-sample vial.

For HPLC, the HPLC protocol I was used.

When 1 μt substrate was added to 499 μl of cleared cell free extract and incubated at 30° C., no conversion of avermectin to 4″-keto-avermectin was observed by HPLC analysis using HPLC protocol I.

However, the possibility of addition of spinach ferredoxin and spinach ferredoxin 5 reductase and NADPH to the cell free extract to restore the biocatalytic activity was explored (see, generally, D. E. Cane and E. I. Graziani, J. Amer. Chem. Soc. 120:2682, 1998).

Accordingly, the following solutions were made:

| Solution | Formula |
|---|---|
| Substrate | 10 mg avermectin were dissolved in 1 ml isopropanol |
| Ferredoxin | 5 mg ferredoxin (from spinach), solution 1-3 mg/ml in Tris/ |
| | HCl-buffer (from Fluka) |
| | or 5 mg ferredoxin (from Clostridium pasteurianum), solution |
| | of 1-3 mg/ml in Tris/HCl-buffer (from Fluka) |
| | or 5 mg ferredoxin (from Porphyra umbilicalis), solution |
| | of 1-3 mg/ml in Tris/HCl-buffer (from Fluka) |
| Ferredoxin | 1 mg freeze-dried ferredoxin reductase (from spinach), |
| Reductase | solution of 3.9 U/mg in 1 ml H₂O (from Sigma) |
| NADPH | 100 mM NADPH in H₂O (from Roche Diagnostics) |

The substrate solution was stored at 4° C., the other solutions were stored at −20° C., and kept on ice when used.

Thus, to 475 μl of cleared cell free extract the following solutions were added: 10 μl ferredoxin, 10 μl ferredoxin reductase and 1 μl substrate. After the addition of substrate to the cells, the mixture was immediately and thoroughly mixed and aerated. Then, 5 μl of NADPH were added and the mixture incubated at 30° C. for 30 min. Then, 1 ml methyl-t-butyl ether was added to the reaction mixture and thoroughly mixed. The mixture was next centrifuged for 2 min. at 14000 rpm, and the methyl-t-butyl ether phase was transferred into a 10 ml flask and evaporated in vacuo by means of a rotary evaporator. The residue was dissolved in 200 μl acetonitrile and transferred into an HPLC-sample vial, and HPLC analysis performed using HPLC protocol I.

Formation of 4″-keto-avermectin was observable by HPLC analysis. Thus, addition of spinach ferredoxin and spinach ferredoxin reductase and NADPH to the cell free extract restored the biocatalytic activity.

Upon injection of a 30 μl sample, a peak appeared at 4.83 min., indicating the presence of 4″-keto-avermectin B la. A mass of 870 D was assigned to this peak by HPLC-mass spectrometry which corresponds to the molecular weight of 4″-keto-avermectin B1a.

Note that when analyzing product formation by HPLC and HPLC-mass spectrometry, in addition to the 4″-keto-avermectin, the corresponding ketohydrate 4″-hydroxy-avermectin was also found giving a peak at 2.12 min. This finding indicated that the P450 monooxygenase converts avermectin by hydroxylation to 4″-hydroxy-avermectin, from which 4″-keto-avermectin is formed by dehydration. Interestingly, when the spinach ferredoxin was replaced by ferredoxin from the bacterium Clostridium pasteurianum or from the red alga Porphyra umbilicalis, the biocatalytic conversion of avermectin to 4″-keto-avermectin still took place, indicating that the enzyme does not depend on a specific ferredoxin for receiving reduction equivalents.

### EXAMPLE IV

### Isolation of a Mutant Streptomyces Strain R-922 With Enhanced Activity

To obtain strains of Streptomyces strain R-922 that have an enhanced ability to regioselectively oxidize avermectin to 4″-keto-avermectin, UV mutants were generated. To do this, spores of Streptomyces strain R-922 were collected and stored in 15% glycerol at −20° C. This stock solution contained 2×10⁹ spores.

The spore stock solution was next diluted and transferred to petri plates containing 10 ml of sterile water, and the suspension was exposed to UV light in a Stratalinker UV crosslinker 2400 (commercially available from Stratagene, La Jolla, Calif.). The Stratalinker UV crosslinker uses a 254-nm light source and the amount of energy used to irradiate a sample can be set in the “energy mode.”

Through experimentation, it was determined that an exposure of 8000 microjoules of UV irradiation (254 nm) was required to kill 99.9% of the spores. This level of UV exposure was used in the mutagenesis.

Surviving UV-mutagenized spores were plated, cultured, and transferred to minimal media. Approximately 0.3-0.4% of the viable spores were determined to be auxotrophic, indicating a good level of mutagenesis in the population.

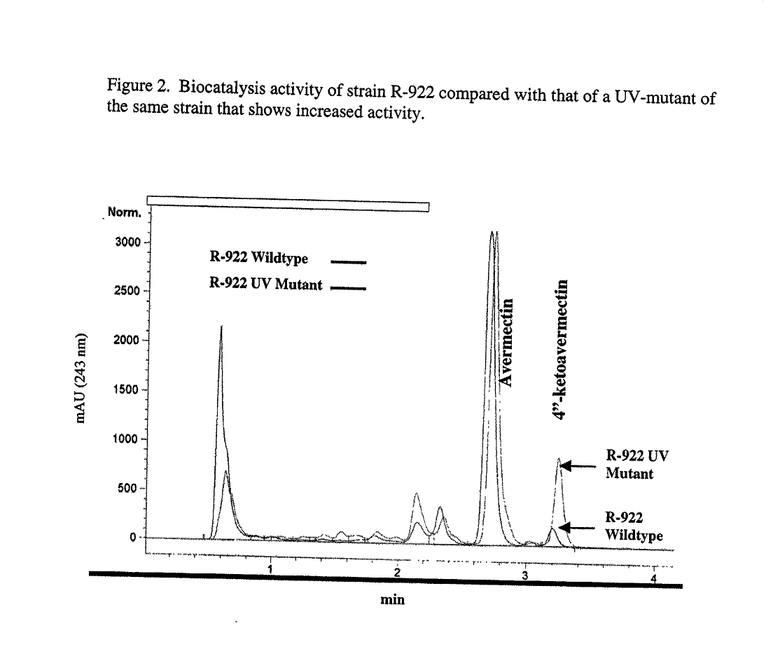

The mutagenized clones were screened for activity in the whole cell biocatalysis assay described in Example II. As shown in FIG. 2, one mutant (“R-922 UV mutant”) showed a two to three fold increase in an ability to regioselectively oxidize avermectin to 4″-keto-avermectin as compared to wild-type strain R-922. Although the gene encoding the P450 monooxygenase responsible for the regioselectively oxidation activity, ema1, is not mutated in the R-922 UV mutant, this mutant nonetheless provides an excellent source for a cell-free extract containing ema1 protein.

### EXAMPLE V

### Isolation of the P450 Monooxygenase from Streptomyces Strain R-922

To enrich the P450 enzyme, 35 ml of active cell free extract were filtered through a 45 μm filter and fractionated by anion exchange chromatography. Anion exchange chromatography conditions were as follows:

| | | | |
|---|---|---|---|
| FPLC instrument: | Ä kta prime (from Pharmacia | | |
| | Biotech) | | |
| FPLC-column: | HiTrap ™Q (5 ml) stacked | | |
| | onto Resource ®Q (6 ml) | | |
| | (from Pharmacia Biotech) | | |
| eluents | buffer A: 25 mM Tris/HCl | | |
| | (pH 7.5) | | |
| | buffer B: 25 mM Tris/HCl | | |
| | (pH 7.5) containing 1 M KCl | | |
| temperature | eluent bottles and fractions in | | |
| | ice bath, | | |
| flow | 3 ml/min | | |
| detection | UV 280 nm | | |
| Pump table: | 0.0 min | 100% A | 0% B |
| linear gradient to | 2.0 min | 90% A | 10% B |
| | 5.0 min | 90% A | 10% B |
| linear gradient to | 30.0 min | 50% A | 50% B |
| linear gradient to | 40.0 min | 0% A | 100% B |
| | 50.0 min | 0% A | 100% B |

Enzyme activity eluted with 35%-40% buffer B. The active fractions were pooled and concentrated by centrifugal filtration through Biomax™ filters with an exclusion limit of 5 kD (commercially available from Millipore Corp., Bedford, Mass.) at 5000 rpm and then rediluted in disruption buffer containing 20% glycerol to a volume of 5 ml containing 3-10 mg/ml protein. This enriched enzyme solution contained at least 25% of the original enzyme activity.

The enzyme was further purified by size exclusion chromatography. Size exclusion chromatography conditions were as follows:

| | |
|---|---|
| FPLC instrument: | Ä kta prime (from Pharmacia Biotech) |
| FPLC-column: | HiLoad 26/60 Superdex ® 200 prep grade (from |
| | Pharmacia Biotech) |
| sample: | 3-5 ml enriched enzyme solution from the anion |
| | chromatography step |
| sample preparation: | filtered through 45 μm filter |
| eluent buffer: | PP-buffer (pH 7.0) + 0.1 M KCl |
| temperature: | 4° C. |
| flow: | 2 ml/min |
| detection: | UV 280 nm |

Enzyme activity eluted between 205-235 ml eluent buffer. The active fractions were pooled, concentrated by centrifugal filtration through Biomax™ filters with an exclusion limit of 5 kD (from Millipore) at 5000 rpm, and rediluted in disruption buffer containing 20% glycerol to form a solution of 0.5-1 ml containing 2-5 mg/ml protein. This enriched enzyme solution contained 10% of the original enzyme activity. This enzyme preparation, when checked for purity by SDS page, (see, generally, Laemmli, U. K., Nature 227:680-685, 1970 and Current Protocols in Molecular Biology, supra) and stained with Coomassie blue, showed one dominant protein band with a molecular weight of 45-50 kD, according to reference proteins of known molecular weight.

### EXAMPLE VI

### Attempted Isolation of P450 Monooxygenase Genes From Streptomyces Strains R-922 and I-1529

Based on results described above that suggested the enzyme from strain R-922 that is responsible for the regiospecific oxidation of avermectin to 4″-keto-avermectin is a P450 monooxygenase, a direct PCR-based approach to clone P450 monooxygenase genes from this strain was initiated (see, generally, Hyun et al., J. Microbiol. Biotechnol. 8(3):295-299, 1998). This approach is based on the fact that all P450 monooxygenase enzymes contain highly conserved oxygen-binding and heme-binding domains that are also conserved at the vii, nucleotide level. PCR primers were designed to prime to these conserved domains and to amplify the DNA fragment from P450 genes using R-922 or I-1529 genomic DNA as a template. The PCR primers used are shown in Table 1.

TABLE 1

| | | SEQ |
|---|---|---|
| | Degen- | ID |
| | eracy | NOs |
| +TL,1 O₂-Binding Domain Primers (5′ to 3′)* | | |
| I A G H E T T | | 43 |
| ATC GCS GGS CAC GAG ACS AC | 8 | 44 |
| V A G H E T T | | 45 |
| GTS GCS GGS CAC GAG ACS AC | 16 | 46 |
| L A G H E T T | | 47 |
| CTS GCS GGS CAC GAG ACS AC | 16 | 48 |
| L L L I A G H E T | | 49 |
| TS CTS CTS ATC GCS GGS CAC GAG AC^{&} | 32 | 50 |
| Heme-Binding Domain Primers (3′ to 5′)* | | |
| H Q C L G Q N L A | | 51 |
| GTG GTC ACG GAS CCS TGC TTG GAS CG^{&} | 8 | 52 |
| F G H G V H Q C | | 53 |
| AAG CCS GTG CCS CAS GTG GTC ACG | 8 | 54 |
| F G F G V H Q C | | 55 |
| AAG GCS AAG CCS CAS GTG GTC ACG | 8 | 56 |
| F G H G I H Q C | | 57 |
| AAG CCS GTG CCS TAG GTG GTC ACG | 4 | 58 |
| F G H G V H F C | | 59 |
| AAG CCS GTG CCS CAS GTG AAG ACG | 8 | 60 |

* The amino acid sequence is shown on the top line and the corresponding nucleotide sequence is shown below on the second line; S = G or C.
^{&}This primer was described by Hyun et al., supra

PCR amplification using any of the primers specific to nucleotide sequences encoding the O₂-binding domain with any of the primers specific to nucleotide sequences encoding the heme-binding domain and genomic DNA from Streptomyces strains R-922 or I-1529 resulted in the amplification of an approximately 350 bp DNA fragment. This is exactly the size that would be expected from this PCR amplification due to the approximately 350 bp separation in P450 genes of the gene segments encoding the O₂-binding and heme-binding sites.

The 350 bp PCR fragments were cloned into the pCR2.1-TOPO TA cloning plasmid (commercially available Invitrogen, Carlsbad, Calif.) and transformed into E. coli strain TOP10 (Invitrogen, Carlsbad, Calif.). Approximately 150 individual clones from strains R-922 and I-1529 were sequenced to determine how many unique P450 gene fragments were represented. Analysis of the sequences revealed that they included 8 unique P450 gene fragments from strain R-922 and 7 unique fragments from 1-1529.

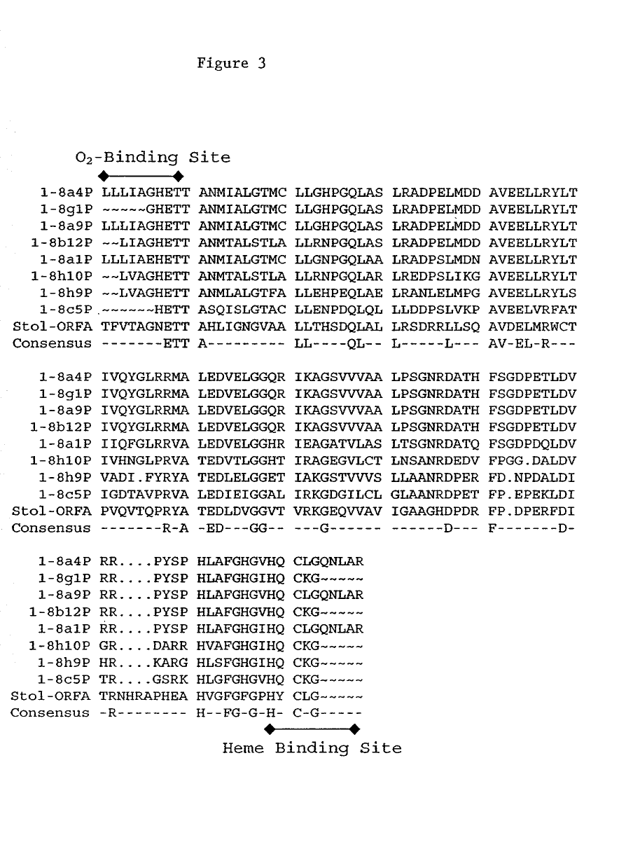

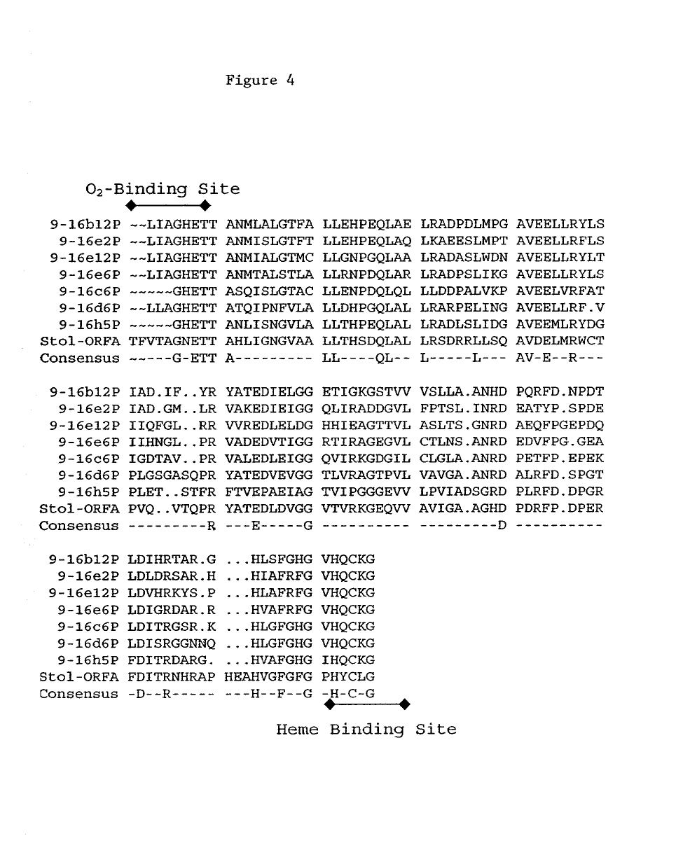

Blast analysis (Altschul et al., J. Mol. Biol. 215:403-410, 1990) demonstrated that all of the unique P450 gene fragments from both the R-922 and I-1529 strains were derived from P450 genes and encoded the region between the O₂-binding and heme-binding domains (see FIG. 3 for strain R-922 and FIG. 4 for strain I-1529).

Next, in order to clone the full-length genes from which the PCR fragments were derived, the DNA fragments cloned by PCR were used as hybridization probes to gene libraries containing genomic DNA from strains R-922 and I-1529. To do this, genomic DNA from the R-922 and I-1529 strains was partially digested with Sau3A I, dephosphorylated with calf intestinal alkaline phosphatase (CIP) and ligated into the cosmid plasmid pPEH215, a modified version of SuperCos 1 (commercially available from Stratagene, La Jolla, Calif.). Ligation products were packaged using the Gigapack III XL packaging extract and transfected into E. coli XL1 Blue MR host cells. Twelve cosmids that strongly hybridized to the PCR-generated P450 gene fragments were identified from the R-922 library, from which three unique P-450 genes were subcloned and sequenced. The hybridizations were performed at high stringency conditions according to the protocol of Church and Gilbert (Church and Gilbert, Proc. Natl. Acad. Sci. USA 81:1991-1995, 1984). In brief, these high stringency conditions include Hybrid Buffer containing 500 mM Na-phosphate, 1 mM EDTA, 7% SDS, 1% BSA; Wash Buffer 1 containing 40 mM Na-phosphate, 1 mM EDTA, 5% SDS, 0.5% BSA; and Wash Buffer 2 containing 40 mM Na-phosphate, 1 mM EDTA, 1% SDS (Note that other high stringency hybridizations conditions are described, for example, in Current Protocols in Molecular Biology, supra.) Nineteen strongly hybridizing cosmids were identified from the I-1529 library, and from these, four unique P-450 genes were subcloned and sequenced.

In yet a further approach to isolate diverse P450 monooxygenase genes from strains R-922 and I-1529, a known P450 gene from another bacterium was used as a hybridization probe to identify cosmid clones containing homologous P450 genes from strains R-922 and I-1529. The epoF P450 gene from Sorangium cellulosum strain So ce90 that is involved in the synthesis of epothilones (Molnar et al., Chem Biol. 7(2):97-109, 2000) was used as a probe in this effort. Using the epoF P450 gene probe, one cosmid was identified from strain R-922 (clone LC), and three were identified from strain I-1529 (clones LA, LB, and EA). In each case, the homologous gene fragment was subcloned and sequenced, and found to code for P450 monooxygenase enzymes.

However, a comparison of the 17 peptide sequences identified in Example VII (below) failed to match any of these cloned genes. Two of the peptide sequences (namely, LVKDDPALLPR (SEQ ID NO: 70) and AVHELMR (SEQ ID NO: 76)) mapped to the region between the O₂ and heme binding domains, and so these should have identified any of the partial gene fragments derived by the PCR approach. Thus, the standard approaches based on the known PCR technique of Hyun et al., supra, and using known P450 genes as hybridization probes failed to identify the gene that encodes the specific P450 monooxygenase responsible for the regioselective oxidation of avermectin. Accordingly, it was determined that additional experimentation was required to isolate the gene encoding the P450 monooxygenase of the invention.

### EXAMPLE VII

### Partial Sequencing of the P450 Monooxygenase from Streptomyces Strain R-922

Partial amino acid sequencing of the P450 monooxygenase from Streptomyces strain R-922 was carried out by the Friedrich Miescher Institute, Basel Switzerland. The protein of the dominant band on the SDS page was tryptically digested and the formed peptides separated and sequenced by mass spectrometry and Edman degradation (see, generally, Zerbe-Burkhardt et al., J. Biol. Chem. 273:6508, 1998). The sequence of the following 17 peptides were found:

| Sequence | Sequence I.D. No. |
|---|---|
| HPGEPNVMDPALITDPFTGYGALR | (SEQ ID NO:61) |
| FVNNPASPSLNYAPEDNPLTR | (SEQ ID NO:62) |
| LLTHYPDISLGIAPEHLER | (SEQ ID NO:63) |
| VYLLGSILNYDAPDHTR | (SEQ ID NO:64) |
| TWGADLISMDPDR | (SEQ ID NO:65) |
| EALTDDLLSELIR | (SEQ ID NO:66) |
| FMDDSPVWLVTR | (SEQ ID NO:67) |
| LMEMLGLPEHLR | (SEQ ID NO:68) |
| VEQIADALLAR | (SEQ ID NO:69) |
| LVKDDPALLPR | (SEQ ID NO:70) |
| DDPALLPR | (SEQ ID NO:71) |
| TPLPGNWR | (SEQ ID NO:72) |
| LNSLPVR | (SEQ ID NO:73) |
| ITDLRPR | (SEQ ID NO:74) |
| EQGPVVR | (SEQ ID NO:75) |
| AVHELMR | (SEQ ID NO:76) |
| AFTAR | (SEQ ID NO:77) |
| FEEVR | (SEQ ID NO:78) |

Alignment of these peptides to a selection of actinomycete P450 monooxygenase sequences indicated that all the peptides were fragments of a single P450 mono-oxygenase.

### EXAMPLE VIII

### Cloning the P450 Monooxygenase Gene from Strain R-922 that Encodes the Enzyme Responsible for the Oxidation of Avermectin to 4″-Keto-Avermectin

PCR primers were designed by reverse translation from the amino acid sequences of several of the peptides derived from the P450 enzyme of strain R-922 (see Example VII and Table 2 below). Each of five forward primers (2aF, 2bF, 3F, 1F, and 7F) was paired with one reverse primer (5R) in PCR reactions with R-922 genomic DNA as a template. In each reaction, a DNA fragment of the expected size was produced.

TABLE 2

| | | | | |
|---|---|---|---|---|
| | | | Expected | |
| | Primer sequence and the amino acid | | size | |
| Primer | sequence to which they were designed* | Degeneracy | (bp)** | SEQ ID NO: |

| | | | | |
|---|---|---|---|---|
| 2aF | P G E D N V M | 64 | 600 | 79 |
| | 5′-CCS GGS GAR CCS AAY GTS ATG-3′ | | | 80 |
| 2bF | A L I T D P F | 32 | 580 | 81 |
| | 5′-GCS CTS ATY ACS GAC CCS TTC-3′ | | | 82 |
| 3F | F M D D S P V W | 32 | 549 | 83 |
| | 5′-TTC ATG GAC GAC WSS CCS GTS TGG-3′ | | | 84 |
| 1F | L N Y D A P D H | 32 | 350 | 85 |
| | 5′-CTS AAY TAY GAC GCS CCS GAC CAC-3′ | | | 86 |
| 7F | V E Q I A D A L | 32 | 300 | 87 |
| | 5′-GTS GAR CAG ATY GCS GAC GCS CTS-3′ | | | 88 |
| 5R | D L I S M D P D | 64 | — | 89 |
| | 3′-CTG GAS TAR WSS TAC CTG GGS CTG-5′ | | 90 | |

* Ambiguity codes: Y = C or T; R = A or G; S = C or G; W = A or T
** Expected size of PCR product when the primer is when paired with primer 5R

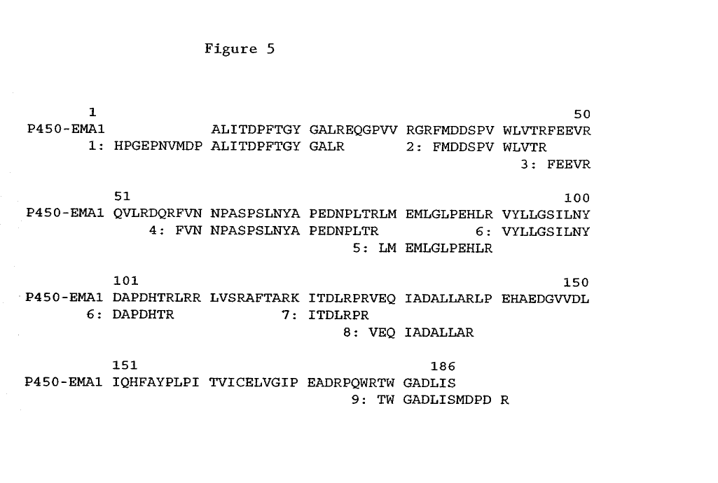

The 580 and 600 bp PCR fragments generated by using primers (2bF and 5R) and (2aF and 5R), respectively, were cloned into the pCR-Blunt II -TOPO cloning plasmid (commercially available from Invitrogen, Carlsbad, Calif.) and transformed into E. coli strain TOP10 (Invitrogen, Carlsbad, Calif.). The inserted DNA fragments were then sequenced. Examination of the sequences revealed that the 600 and 580 bp fragments were identical in the 580 bp of sequence that they have in common. Also, there was a perfect match between the deduced amino acid sequence derived from the nucleotide sequence of the 600 bp and 580 bp fragments and the amino acid sequences of peptides isolated from the purified P450_{Ema1} enzyme that aligned in this region of the isolated gene (see FIG. 5). This result strongly suggested that the gene fragments isolated in these clones are derived from the gene that encodes the P450_{Ema1} enzyme that is responsible for the oxidation of avermectin to 4″-keto-avermectin.

The 600 bp PCR fragment produced using primers 2aF (SEQ ID NO: 80) and 5R(SEQ ID No: 90) was used as a hybridization probe to a cosmid library of genomic DNA isolated from strain R-922 (cosmid library described in Example VI). Two cosmids named pPEH249 and pPEH250 were identified that hybridized strongly with the probe. The portion of each cosmid encoding the P450 enzyme was sequenced and the sequences were found to be identical between the two cosmids. The complete coding sequence of the ema1 gene was identified (SEQ ID NO: 1). The amino acid sequence of all peptide fragments from P450_{Ema1} matched perfectly with the deduced amino acid sequence from the ema1 gene (see FIG. 5). Comparison of the deduced amino acid sequence of the protein encoded by the ema1 gene using BLASTP (Altschul et al., supra) determined that the closest match in the databases is to a P450 monooxygenase from S. thermotolerans that has a role in the biosynthesis of carbomycin (Arisawa et al., Biosci. Biotech. Biochem. 59(4):582-588, 1995) and whose identity with ema1 is only 49% (Identities=202/409 (49%), Positives=271/409 (65%), Gaps=2/409 (0%)). In the Blast analysis, the following settings were employed:

BLASTP 2.0.10

| | | |
|---|---|---|
| Lambda | K | H |

| | | |
|---|---|---|
| 0.322 | 0.140 | 0.428 |

Gapped

| | | |
|---|---|---|
| Lambda | K | H |

| | | |
|---|---|---|
| 0.270 | 0.0470 | 0.230 |

Matrix: BLOSUM62
Gap Penalties: Existence: 11, Extension: 1
Number of Hits to DB: 375001765
Number of Sequences: 1271323
Number of extensions: 16451653
Number of successful extensions: 46738
Number of sequences better than 10.0: 2211
Number of HSP's better than 10.0 without gapping: 628
Number of HSP's successfully gapped in prelim test: 1583
Number of HSP's that attempted gapping in prelim test: 43251
Number of HSP's gapped (non-prelim): 2577
length of query: 430
length of database: 409,691,007
effective HSP length: 55
effective length of query: 375
effective length of database: 339,768,242
effective search space: 127413090750
effective search space used: 127413090750

A similar comparison of the nucleotide sequences of these two genes demonstrated that they are 65% identical at the nucleotide level. These results demonstrate that P450_{Ema1} is a new enzyme.

### EXAMPLE IX

### Heterologous Expression of the ema1 Gene in Strentomyces lividans Strain ZX7

The coding sequence of the ema1 gene was fused to the thiostrepton-inducible promoter (tipA) (Murakami et al., J. Bacteriol. 171:1459-1466, 1989). The tipA promoter was derived from plasmid pSIT151 (Herron and Evans, FEMS Microbiology Letters 171:215-221, 1999).

The fusion of the tipA promoter and the ema1 coding sequence was achieved by first amplifying the ema1 coding sequence with the following primers to introduce a PacI cloning site at the 5′ end and a PmeI compatible end on the 3′ end.

Forward Primer: The underlined sequence is a PacI recognition sequence; the sequence in bold-face type is the start of the coding sequence of ema1.

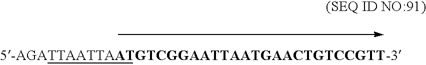

Reverse Primer: The underlined sequence is half of a PmeI recognition sequence; the bold-face type sequence is the reverse complement of the ema1 translation stop codon followed by the 3′ end of the ema1 coding sequence.

(SEQ ID NO:92)

5′-AAACTCACCCCAACCGCACCGGCAGCGAGTTC-3″

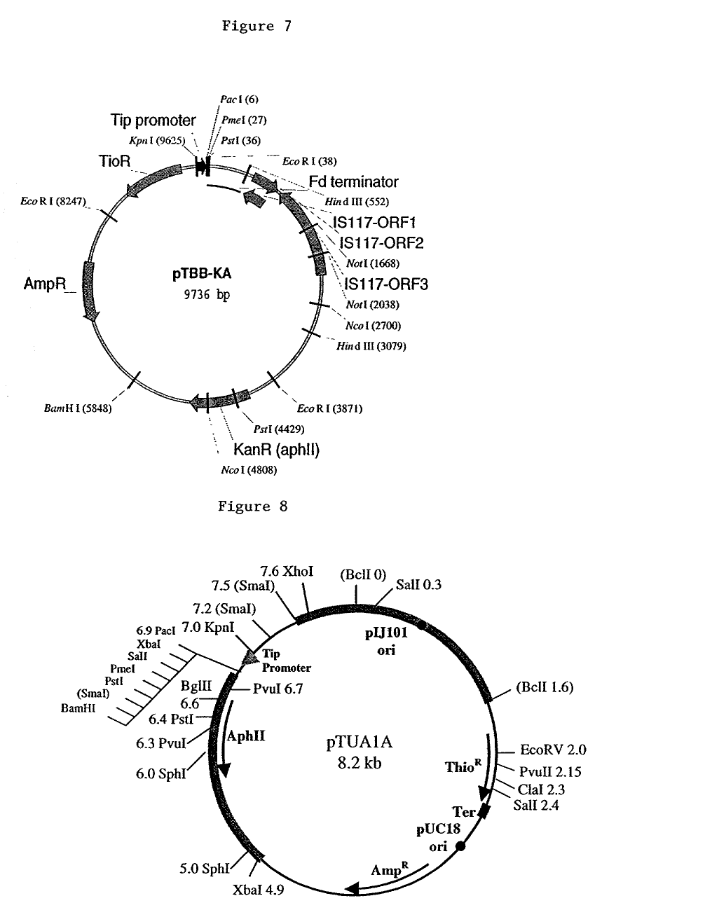

The PacI-digested PCR fragment containing the ema1 coding sequence was cloned into plasmid pTBBKA (see FIG. 7) that was restricted (i.e., digested) with PacI and PmeI, and the ligated plasmid transformed into E. coli. Four clones were sequenced. Three of the four contained the complete and correct ema1 coding sequence. The fourth ema1 gene clone contained a truncated version of the ema1 gene. The full-length ema1 gene encodes a protein that begins with the amino acid sequence MSELMNS (SEQ ID NO: 93). The truncated gene encodes a protein that lacks the first 4 amino acids and begins with the second methionine residue. This gene has been named ema1A. The nucleotide and amino acid sequence of ema1A are provided as SEQ ID NO: 33 and SEQ ID NO: 34, respectively. The ema1 and ema1A genes in these plasmids, pTBBKA-ema1 and pTBBKA-ema1A, are in the correct juxtaposition with the tipA promoter to cause expression of the genes from this promoter.

Plasmid pTBBKA contains a gene from the Streptomyces insertion element IS117 that encodes an integrase that catalyzes site-specific integration of the plasmid into the chromosome of Streptomyces species (Henderson et al., Mol. Microbiol. 3:1307-1318, 1989 and Lydiate et al., Mol. Gen. Genet. 203:79-88, 1986). Since plasmid pTBBKA has only an E. coli replication origin and contains a mobilization site, it can be transferred from E. coli to Streptomyces strains by conjugation where it will not replicate. However, it is able to integrate into the chromosome due to the IS 117 integrase and Streptomyces clones containing chromosomal integrations can be selected by resistance to kanamycin due to the plasmid-borne kanamycin resistance gene.

The ema1 coding sequence was also cloned into other plasmids that are either replicative in Streptomyces or, like pTBBKA, integrate into the chromosome upon introduction into a Streptomyces host. For example, ema1 was cloned into plasmid pEAA, which is similar to plasmid pTBBKA but the KpnI/PacI fragment containing the tipA promoter was replaced with the ermE gene promoter (Schmitt-John and Engels, Appl Microbiol Biotechnol. 36(4):493-498, 1992). In addition, pEAA does not contain the kanamycin resistance gene. The ema1 gene was cloned into pEAA as a PacI/PmeI fragment to create plasmid pEAA-ema1 in which the ema1 gene is expressed from the constitutive ermE promoter.

Plasmid pTUA1A is a Streptomyces-E.coli shuttle plasmid (see FIG. 8) that contains the tipA promoter. The ema1 gene was also cloned into the PacI/PmeI sites in plasmid pTUA1A to create plasmid pTUA-ema1.

The ema1 A gene fragment was also ligated as a PacI/PmeI fragment into plasmids pTUA1A, and pEAA in the same way as the ema1 gene fragment to create plasmids pTUA-ema1A, and pEAA-ema1 A, respectively.

The pTBBKA, pTUA1A, and pEAA-based plasmids containing the ema1 or ema1A genes were introduced into S. lividans ZX7 and in each case transformants were obtained and verified (S. lividans l strains ZX7::pTBBKA-ema1 or ema1A, ZX7 (pTUA-ema1 or -ema1 A), and ZX7::pEAA-ema1 or -ema1A, respectively).

Wild-type Streptomyces lividans strain ZX7 was tested and found to be incapable of the oxidation of avermectin to 4″-keto-avermectin. Transformed S. lividans strains ZX7::pTBBKA-ema1, ZX7::pTBBKA-ema1A, ZX7 (pTUA-ema1), ZX7 (pTUA-ema1A), ZX7::pEAA-ema1, and ZX7::pEAA-ema1A were each tested for the ability to oxidize avermectin to 4″-keto-avermectin using resting cells. To do this, the whole cell biocatalysis assay described above (including analysis method) was performed. Note that for the whole cell biocatalysis assay, transformed Streptomyces lividans, like strain R-922, was grown in PHG medium and, again like strain R-922, had a reaction time of 16 hours (i.e., during which time the 500 mg transformed Streptomyces lividans wet cells in 10 ml of 50 mM potassium phosphate buffer, pH 7.0, were shaken at 160 rpm at 28° C. in the presence of 15 μl of a solution of avermectin in isopropanol (30 mg/ml)).

In the presence of the inducer, thiostrepton (5 ug/ml), the ema1- or ema1A-containing strains ZX7::pTBBKA-ema1, ZX7::pTBBKA-ema1A, ZX7 (pTUA-ema1), ZX7 (pTUA-ema1A) were found to oxidize avermectin to 4″-keto-avermectin as evidenced by the appearance of the oxidized 4″-keto-avermectin compound (see Table 3).

TABLE 3

| | % Conversion of Avermectin | |
|---|---|---|
| Strain | 2 hour | 16 hour |
| Streptomyces lividans ZX7 + | | |
| Plasmid¹ | | |
| None | 0 | 0 |
| pTBBKA-ema1A | 0.5 ± 0.059 | 1.17 ± 0.112 |
| pTBBKA-ema1 | 0.21 ± 0.0.356 | 0.65 ± 0.079 |
| pTUA-ema1 | 20.96 ± 1.044 | 42.0 ± 2.5 |
| pEAA-ema1 | 3.0 ± 0.232 | 24.1 ± 0.358 |
| pTBBKA-ema2 | 4.79 ± 0.096 | 9.57 ± 0.423 |
| pTUA-ema2 | 0.77 ± 0.138 | 2.05 ± 0.537 |
| pEAA-ema2 | 0.0 | 1.73 ± 3.00 |
| pTBBKA-ema1/fd233 | 8.89 ± 0.720 | 30.99 ± 0.880 |
| pTUA-ema1/fd233 | 23.29 ± 0.854 | 61.2 ± 3.548 |
| pEAA-ema1/fd233 | 8.26 ± 0.845 | 10.66 ± 0.858 |
| pTUA-ema2/fd233 | 1.85 ± 0.861 | 6.40 ± 1.918 |
| Pseudomonas putida S12 + | | |
| Plasmid | | |
| None | | 0 |
| pRK-ema1 | ND² | 18 |
| pRK-ema1/fd233 | ND | 32 |

¹pTBBKA = IS117 integrase, tipA promoter; pTUA = replicative plasmid, tipA promoter; pEAA = IS117 integrase, ermE promoter;
²Not Determined

These results conclusively demonstrate that the P450_{Ema1} enzyme encoded by the ema1 gene is responsible for the oxidation of avermectin to 4″-keto-avermectin in S. tubercidicus strain R-922. Furthermore, the data demonstrates that the ema1A gene that is 4 amino acids shorter on the N-terminus than the native ema1 gene also encodes an active P450_{Ema1} enzyme.

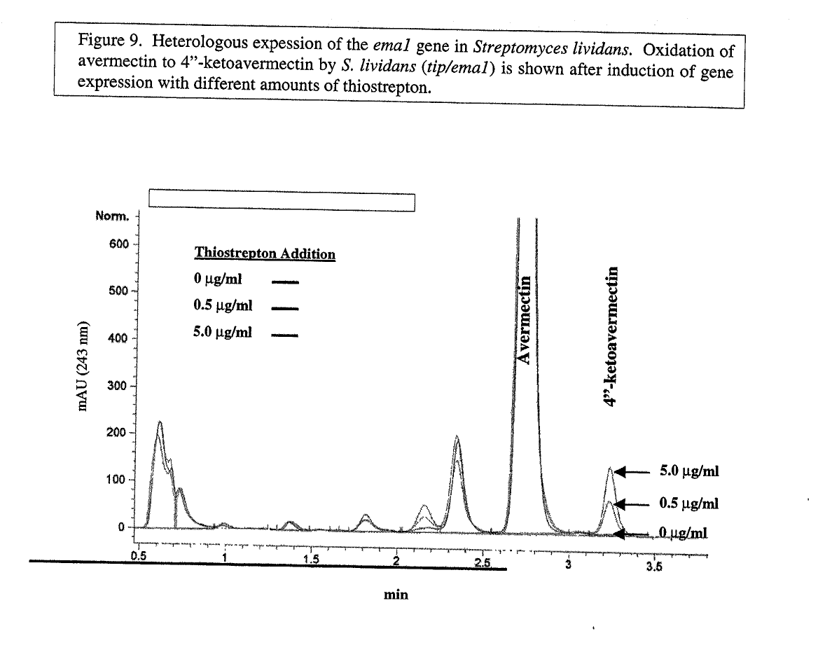

As can be seen in FIG. 9, oxidation of avermectin to 4″-keto-avermectin by S. lividans strain ZX7::pTBBKA-ema1, as detected by HPLC analysis, is variable depending upon the amount of thiostrepton used to induce expression of ema1. Note that S. lividans strains ZX7::pEAA-ema1 and ZX7::pEAA-ema1A (see Table 3) demonstrated this oxidation activity in the absence of thiostrepton since in these strains the ema1 or ema1A genes are expressed from the ermE promoter that does not require induction.

### EXAMPLE X

### Isolation of an ema1-Homolosous Gene From Streptomyces tubercidicus Strain I-1529

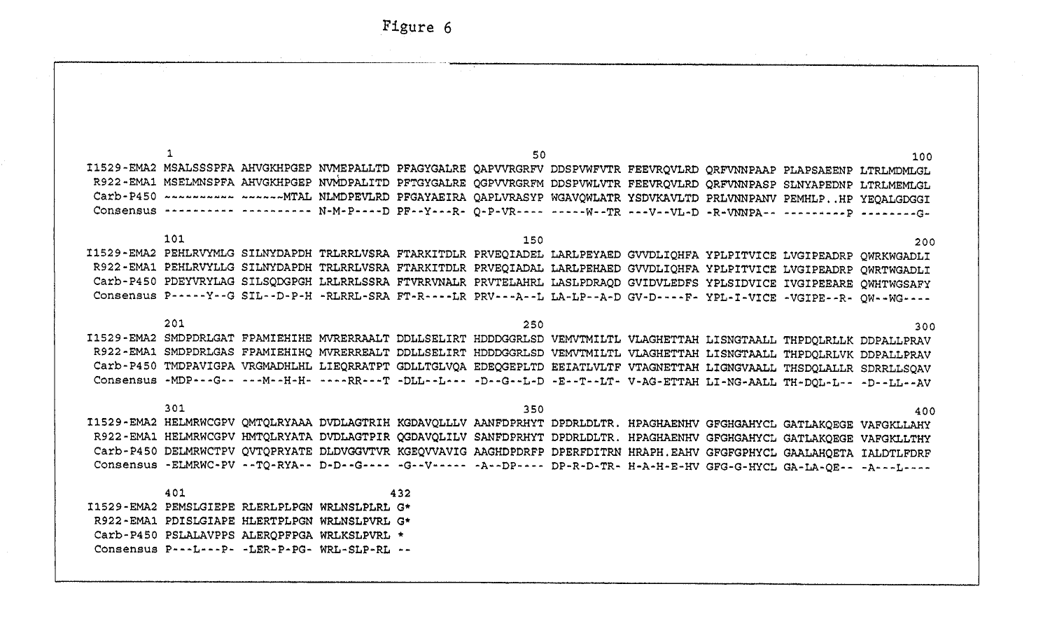

Streptomyces tubercidicus strain I-1529 was also found to be active in biocatalysis of avermectin to form the 4″-keto-avermectin derivative. The cosmid library from strain I-1529, described in Example VI, was probed at the high stringency conditions of Church and Gilbert (Church and Gilbert, Proc. Natl. Acad. Sci. USA 81:1991-1995, 1984) with the 600 bp ema1 PCR fragment produced using primers 2aF (SEQ ID NO: 80) and 5R (SEQ ID NO: 90) described previously to identify clones containing the ema1 homolog from strain I-1529. Three strongly hybridizing cosmids were identified. The P450 gene regions in two of the cosmids, pPEH252 and pPEH253, were sequenced and found to be identical. Analysis of the DNA sequence revealed the presence of a gene with high homology to the ema1 gene of strain R-922. FIG. 6 shows a comparison of the deduced amino acid sequence of Ema2 (i.e., P450_{Ema2}), Ema1 (i.e., P450_{Ema1}), and a P450 monooxygenase from Streptomyces thermotolerans that is involved in the biosynthesis of carbomycin (Carb-450) (GenBank Accession No. D30759).

The gene from Streptomyces tubercidicus strain I-1529, named ema2, encodes an enzyme with 90% identity at the amino acid level and 90.6% identity at the nucleotide level to the P450_{Ema1} enzyme. The nucleotide sequence of the ema2 gene and the deduced amino acid sequence of P450_{Ema2} are provided in SEQ ID NO: 3 and SEQ ID NO: 4, respectively.

The ema2 coding sequence was cloned in the same manner as the ema1 and ema1A genes into plasmids pTBBKA, pTUA1A, and pEAA such that the coding sequence was functionally fused to the tipA or ermE promoter in these plasmids. The resulting plasmids, pTBBKA-ema2, pTUA-ema2, and pEAA-ema2 were transferred from E. coli to S. lividans ZX7 by conjugation to create strains ZX7::TBBKA-ema2 and ZX7 (pTUA-ema2), and ZX7::pEAA-ema2 containing the ema2 gene integrated into the chromosome or maintained on a plasmid.

Strains ZX7::TBBKA-ema2, ZX7 (pTUA-ema2), and ZX7::pEAA-ema2 were next tested for the ability to oxidize avermectin to 4″-keto-avermectin. The ema2 gene was also shown to provide biocatalysis activity, although at a lower level compared to the ema1 gene (see Table 3).

These results demonstrate that the ema2 gene from S. tubercidicus strain I-1529 also encodes a P450 enzyme (P450_{Ema2}) capable of oxidizing avermectin to 4″-keto-avermectin.

### EXAMPLE XI

### Characterization of ema1 Homologs From Other Biocatalysis Strains

Seventeen Streptomyces sp. strains, including strains R-922 and I-1529, were identified that are capable of catalyzing the regiospecific oxidation of the 4″-carbinol of avermectin to a ketone. Next, the isolation and characterization of the genes encoding the biocatalysis enzyme from all of these strains was accomplished.

To do this, genomic DNA was isolated from the strains and was evaluated by restriction with several restriction endonucleases and Southern hybridization with the ema1 gene. A specific restriction endonuclease was identified for each DNA that would generate a single DNA fragment of a defined size to which the ema1 gene hybridizes. For each strain, there was only one strongly hybridizing DNA fragment, thus suggesting that other P450 genes were not detected under the high stringency hybridization conditions used in these experiments. Each DNA was digested with the appropriate restriction endonuclease, and the DNA was subjected to agarose gel electrophoresis. DNA in a narrow size range that included the size of the ema1-hybridizing fragment was excised from the gel. The size-selected DNA was ligated into an appropriate cloning plasmid and this ligated plasmid was used to transform E. coli. The E. coli clones from each experiment were screened by colony hybridization with the ema1 gene fragment to identify clones containing the ema1-homologous DNA fragment.

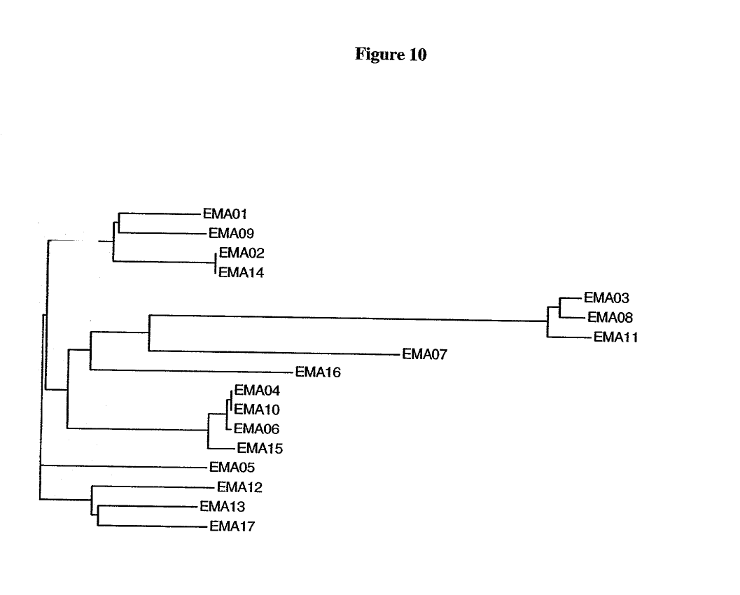

The nucleotide sequence of the cloned DNA in each ema1-homologous clone was determined and examined for the presence of a gene encoding a P450 enzyme with homology to ema1. In this way, ema1-homologous genes were isolated from 14 of the 15 other active strains. The nucleotide and deduced amino acid sequences of these are referenced in Table 4 as SEQ ID NOS: 5-32 and 94-95. A diagram of the relationship of these enzymes in the form of a phylogenetic tree is shown in FIG. 10. This phylogenetic tree was generated using the commercially available GCG Wisconsin software program version 1.0 (Madison, Wis.).

TABLE 4

| | | | SEQ ID NO |
|---|---|---|---|
| | | | (nucleotide and |
| | | | amino acid, |
| Strain Number | Gene | Classification | respectively) |
| R-0922 | ema1 | Strept. tubercidicus | 1 and 2 |
| I-1529 | ema2 | Strept. tubercidicus | 3 and 4 |
| 1053 | ema3 | Streptomyces rimosus | 5 and 6 |
| R-0401 | ema4 | Streptomyces lydicus | 7 and 8 |
| I-1525 | ema5 | Streptomyces sp. | 9 and 10 |
| DSM-40241 | ema6 | Strept. chattanoogensis* | 11 and 12 |
| IHS-0435 | ema7 | Streptomyces sp. | 13 and 14 |
| C-00083 | ema8 | Streptomyces albofaciens | 15 and 16 |
| MAAG-7479 | ema9 | Streptomyces platensis | 17 and 18 |
| A/96-1208710 | ema10 | Strept. kasugaensis | 19 and 20 |
| R-2374 | ema11 | Streptomyces rimosus | 21 and 22 |
| MAAG-7027 | ema12 | Strept. tubercidicus | 23 and 24 |
| Tue-3077 | ema13 | Streptomyces platensis | 25 and 26 |
| I-1548 | ema14 | Streptomyces platensis | 27 and 28 |
| NRRL-2433 | ema15 | Strept. lydicus | 29 and 30 |
| MAAG-0114 | ema16 | Streptomyces lydicus | 31 and 32 |
| DSM-40261 | ema17 | Streptomyces tubercidicus | 94 and 95 |

*This strain was shown to be in the chattanoogensis species by 16s rDNA analysis; however, classical taxonomic methods used by the German culture collection (DSMZ) showed it to be saraceticus.

### EXAMPLE XII

### Construction of His-tagged ema1 and ema1 Homologs to Facilitate Enzyme Purification

In order to purify the P450_{Ema1} enzyme and the P450 enzymes encoded by the ema1 homologs from other biocatalysis strains, each of the P450 genes was cloned into the E. coli expression plasmid pET-28b(+) (commercially available from Novagen, Madison, Wis.). The pET-28 plasmids are designed to facilitate His-tag fusions at either the N-, or C-terminus and to provide strong expression of the genes in E. coli from the T7 phage promoter. In many cases, the coding sequence of the ema genes begins with the sequence ATGT. These genes were amplified by PCR such that the primers on the 5′ end incorporated a PciI recognition site (5′ ATATGT 3′) at the 5′ terminus. The last four bases of the PciI site correspond to the ATGT at the beginning of the ema gene coding sequence.

PCR primers at the 3′ end of the genes were designed to remove the translation stop codon at the end of the ema gene coding sequence and to add an XhoI recognition site to the 3′ terminus. The resulting PCR fragments were restricted with PciI and XhoI to generate PciI ends at the 5′ termini and XhoI ends at the 3′ termini, thereby facilitating cloning of the fragments into pET-28b(+) previously restricted with NcoI and XhoI. Since PciI and NcoI ends are compatible, the fragments were cloned into pET-28b(+) in the proper orientation to the T7 promoter and ribosome binding site in the plasmid to provide expression of the genes.

At the 3′ end of each ema gene, the coding sequence was fused in frame at the XhoI site to the His-tag sequence followed by a translation stop codon. This results in the production of an Ema enzyme with six histidine residues added to the C-terminus to facilitate purification on nickel columns.

In the case of ema genes in which the ATG translation initiation codon is not followed by a T nucleotide, the ema genes were amplified by PCR using a different strategy for the 5′ end. The primers at the 5′ end were designed to incorporate a C immediately preceding the ATG translation initiation codon and the primers at the 3′ end were the same as described above. The PCR fragments that were amplified were restricted with XhoI to create an XhoI end at the 3′ -terminus and the 5′ end was left as a blunt end. These fragments were cloned into pET-28b(+) that had been restricted with NcoI, but the NcoI ends were made blunt-ended by treatment with mung bean exonuclease, and restricted with XhoI.

In this manner, the ema genes were cloned into pET-28b(+) to create a functional fusion with the T7 promoter and the His-tag at the C-terminus as described previously. All His-tagged ema genes were sequenced to ensure that no errors were introduced by PCR.

Large amounts of the His-tagged P450_{Ema1} and P450_{Ema2} enzymes were isolated and purified by standard protocols. E. coli strain BL21 DE3 (commercially available from Invitrogen; Carlsbad, Calif.) containing the T7 RNA polymerase gene under the control of the inducible tac promoter and the appropriate pET-28/ema plasmid was cultured and the cells were harvested and lysed. The lysates were applied to Ni-NTA columns (commercially available from Qiagen Inc., Valencia, Calif.) and the protein was purified according to the procedure recommended by the manufacturer.

Purified His-tagged P450_{Ema1} and P450_{Ema2} were highly active in in vitro activity assays as evidenced by a high rate of conversion of avermectin to 4″-keto-avermectin.

### EXAMPLE XIII

### Expression of ema1 in Pseudomonas

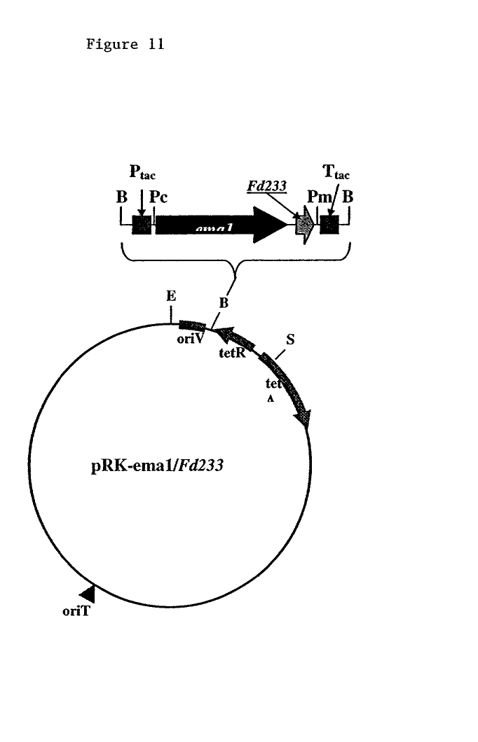

The ema1 gene constructs were next introduced into P. putida (wildtype P. putida commercially available from the American Type Culture Collection, Manassas, Va.; ATCC Nos. 700801 and 17453). The ema1 and ema1/fd233 gene fragments were cloned as PacI/PmeI fragments into the plasmid pUK21 (Viera and Messing, Gene 100:189-194, 1991). The fragments were cloned into a position located between the tac promoter (P_{tac}) and terminator (T_{tac}) on pUK21 in the proper orientation for expression from the tac promoter. The P_{tac-ema}1-T_{tac} and P_{tac}-ema1/fd233-T_{tac} gene fragments were removed from pUK21 as Bg1II fragments and these were cloned into the broad host-range, transmissible plasmid, pRK290 (Ditta et al., Proc. Natl. Acad. Sci. USA 77:7347-7351, 1980) to create plasmids pRK-ema1 and pRK-ema1/fd233 (FIG. 11). These plasmids were introduced into P. putida strains ATCC 700801 and ATCC 17453 by conjugal transfer from E. coli hosts by standard methodology (see, e.g., Ditta et al., Proc. Natl. Acad. Sci. USA 77:7347-7351, 1980).

P. putida ATCC 700801 and ATCC 17453 containing plasmids pRK-ema1 or pRK-ema1/fd233 were tested for the ability to catalyze the oxidation of avermectin. The results shown in Table 3 demonstrate that these strains are able to catalyze this reaction.

### EXAMPLE XIV

### Identification of Genes Encoding Ferredoxins That Are Active With the P450_{Ema1} Monooxygenase

P450 monooxygenases require two electrons for each hydroxylation reaction catalyzed (Mueller et al., “Twenty-five years of P450_{cam} research: Mechanistic Insights into Oxygenase Catalysis.” Cytochrome P450, 2^{nd} Edition, P. R. Ortiz de Montellano (ed.), pp. 83-124; Plenum Press, NY 1995). These electrons are transferred to the P450 monooxygenase one at a time by a ferredoxin. The electrons are ultimately derived from NAD(P)H and are passed to the ferredoxin by a ferredoxin reductase. Specific P-450 monooxygenase enzymes have a higher activity when they interact with a specific ferredoxin. In many cases, the gene encoding a ferredoxin that interacts specifically with a given P450 monooxygenase is located adjacent to the gene encoding the P450 enzyme.

As described above, in addition to the ema1 gene, four P450 genes from strain R-922 and seven P450 genes from strain I-1529 (see Example VI) were isolated and sequenced. In some of these, there was sufficient sequence information about the DNA flanking the P-450 genes to look for the presence of associated ferredoxin genes. By this approach, two unique ferredoxin genes were identified from each of the two strains. Ferredoxin genes fd229 and fd230 were identified from strain R-922, and fd233 and fdEA were identified from strain I-1529. In addition, a ferredoxin reductase gene was found to reside adjacent to the fdEA gene from strain I-1529. In order to test the biological activity of each of these ferredoxins in combination with P450_{Ema1}, each individual ferredoxin gene was amplified by PCR to produce a gene fragment that included a blunt 5′-end, the native ribosome-binding site and ferredoxin gene coding sequence, and a PmeI restriction site on the 3′-end. Each such ferredoxin gene fragment was cloned into the PmeI site located 3′ to the ema1 gene in plasmid pTUA-ema1. In this way, artificial operons consisting of the ema1 gene and one of the ferredoxin genes operably linked to a functional promoter were created.

In the case of the fdEA ferredoxin gene in which a ferredoxin reductase gene, freEA, was found to be located adjacent to the fdEA gene, a DNA fragment containing both the fdEA and freEA genes was generated by a similar PCR strategy. This gene fragment was also cloned in the PmeI site of plasmid pTUA-ema1 as described for the other ferredoxin genes.

Each ema1-ferredoxin gene combination was tested for biological activity by introduction of the individual ema1-ferredoxin gene plasmids into S. lividans strain ZX7. The biocatalysis activity derived from each plasmid in S. lividans was determined. Of the four different constructs, only the ferredoxin gene fd233 derived from strain I-1529 provided increased activity when compared to the expression of ema1 alone in the same plasmid and host background (see Table 3). The pTUA-ema1/fd233 plasmid in S. lividans gave approximately 1.5 to 3 fold higher activity compared to the pTUA-ema1 plasmid. The other three plasmids containing the other ferredoxin genes provided results essentially the same as the plasmid with only the ema1 gene. Likewise, the pTUA-ema1/fdEA/freEA plasmid did not yield results different from those of pTUA-ema1. The nucleotide and deduced amino acid sequences of the fd233 gene are shown in SEQ ID NOs: 35 and 36, respectively.

A BLAST analysis of the nucelotide and amino acid sequences of fd233 revealed that the closest matches were to ferredoxins from S. coelicolor (GenBank Accession AL445945) and S. lividans (GenBank Accession AF072709). At the nucleotide level, fd233 shares 80 and 79.8% identity with the ferredoxin genes from S. coelicolor and S. lividans, respectively. At the peptide level, fd233 shares 79.4 and 77.8% identity with the ferredoxins from S. coelicolor and S. lividans, respectively.

Since fd233 is derived from strain I-1529 and ema1 is from strain R-922, the proteins encoded by the two genes cannot interact with each other in nature. In an approach designed to identify a ferredoxin gene from strain R-922 that is homologous to the fd233 gene and that might encode a ferredoxin that interacts optimally with the P450 _{Ema1}, the fd233 gene was used as a hybridization probe to a gene library of DNA from strain R-922. A strongly hybridizing cosmid, pPEH232, was identified and the hybridizing DNA was cloned and sequenced. Comparison of the deduced amino acid sequences from fd233 and the ferredoxin gene on cosmid pPEH232,fd232, revealed that they differed in only a single amino acid.

In a similar manner, plasmid pTUA-ema1-fd232 was constructed and tested in S. lividans ZX7. This plasmid gave similar results as those obtained with plasmid pTUA-ema1-fd233 (see Table 3). The nucleotide and deduced amino acid sequences of fd232 are shown in SEQ ID NOs: 37 and 38, respectively.

The ema1-fd233 operon was also subcloned, as a PacI-PmeI fragment, into pTBBKA and pEAA that had been digested with the same restriction enzymes. S. lividans ZX7::pTBBKA-ema1-fd233, and S. lividans ZX7::pEAA-ema1-fd233 were tested in the avermectin conversion assay and found to have higher activities than the strains harboring the ema1 gene alone in the comparable plasmids (see Table 3).

### EXAMPLE XV

### Heterologous Expression of P450_{Ema1} and P450_{Ema2} in Other Cells

The expression constructs pRK-ema1 (Example XIII) and pRK-ema2 (created in a way analogous to that described in Example XIII for pRK-ema1) were mobilized by conjugation into three fluorescent soil Pseudomonas strains. Conjugation was performed according to standard methods (see, e.g., Ditta et al., Proc. Natl. Acad. Sci. USA 77:7347-7351, 1980). The strains were: P. fluorescens MOCG134, P. fluorescens Pf-5, and P. fluorescens CHAO. Standard resting cell assays for the conversion of avermectin to 4″-ketoavermectin were conducted for each of the transconjugants. For strains Pf-5 and CHAO, the levels of conversion were below the detection limit. Strain MOCG134 yielded 3% conversion for ema1 and 5% for ema2.

In addition, the constructs listed in the Table 5 were introduced into Streptomyces avermitilis MOS-0001 by protoplast-mediated transformation (see, e.g., Kieser, T.; Bibb, M. J.; Buttner, M. J.; Chater, K. F.; Hopwood, D. A. (eds.): Practical Streptomyces Genetics. The John Innes Foundation, Norwich (England), 2000); Stutzman-Engwall, K. et al. (1999) Streptomyces avermitilis gene directing the ratio of B₂:B₁ avermectins, WO 99/41389).

TABLE 5

| Construct | % Conversion of avermectin, 16 hrs |
|---|---|
| None | 0 |
| pTBBKA-ema1 | 10.90 +/− 3.48 |
| pTUA-ema1 | 5.326 +/− 2.19 |
| pEAA-ema1 | 6.74 +/− 0.08 |
| pTBBKA-ema1A/fd233 | 28.50 +/− 0.20 |
| pTUA-ema1A/fd233 | 23.97 +/− 5.95 |

Wild-type Str. avermitilis MOS-0001 was tested and found to be incapable of the oxidation of avermectin to 4″-ketoavermectin.

Transformed S. avermitilis strains MOS-0001::pTBBKA-ema1, MOS-0001 (pTUA-ema1), MOS-0001::pEAA-ema1, MOS-0001::pTBBKA-ema1A-fd233, and MOS-0001 (pTUA-ema1A-fd233) were each tested for their ability to oxidize avermectin to 4″-keto-avermectin using resting cells. To do this, the whole cell biocatalysis assay described above (including analysis method) was performed. Note that for the whole cell biocatalysis assay, transformed Streptomyces avermitilis, like strain R-922, was grown in PHG medium and, again like strain R-922, had a reaction time of 16 hours (i.e., during which time the 500 mg transformed Streptomyces avermitilis wet cells in 10 ml of 50 mM potassium phosphate buffer, pH 7.0, were shaken at 160 rpm at 28° C. in the presence of 15 μl of a solution of avermectin in isopropanol (30 mg/ml)).

As shown in Table 5, in the presence of the inducer, thiostrepton (5 μg/ml), the ema1- or ema1A-fd233-containing strains MOS-0001::pTBBKA-ema1, MOS-0001::pTBBKA-ema1A-fd233, MOS-0001 (pTUA-ema1), MOS-0001 (pTUA-ema1A-fd233) were found to oxidize avermectin to 4″-keto-avermectin as evidenced by the appearance of the oxidized 4″-keto-avermectin compound. Note that the S. avermitilis strain MOS-0001::pEAA-ema1 demonstrated this oxidation activity in the absence of thiostrepton since in this strain the ema1 gene is expressed from the ermE promoter that does not require induction.

Thus, expression of the ema1 P450 monooxygenase gene in various Streptomyces and Pseudomonas strains provided recombinant cells that were able to convert avermectin to 4″-ketoavermectin in resting cell assays.

Next, expression and activity of P450_{Ema1} monooxygenase was tested in E. coli. To do this, the ema1 gene was cloned into the E. coli expression plasmid pET-28b(+) (commercially available from Novagen, Madison, Wis.) as described previously. E. coli strain BL21 DE3 (commercially available from Invitrogen; Carlsbad, Calif.) that contains the T7 RNA polymerase gene under control of the inducible tac promoter and the pET-28/ema1 plasmid was cultured in 50 ml LB medium containing 5 mg/l kanamycin in a 250-ml flask with one baffle, for 16 hours at 37° C., with shaking at 130 rpm. 0.5 ml of this culture was used to inoculate 500 ml LB medium with 5 mg/l kanamycin in a 2-liter flask with one baffle, and the culture was incubated for 4 hours at 37° C. followed by 4 hours and 30° C., with shaking at 130 rpm throughout. The cells were harvested by centrifugation, washed in 50 mM potassium phosphate buffer, and centrifuged again.

For the resting cell assays, 90 mg wet cells were weighed into deep-well plates in triplicate and resuspended in 0.5 ml 50 mM potassium phosphate buffer. For cell-free extracts, 4 grams wet cells in 8 ml disruption buffer were disrupted in French press.

For the resting cell assays, 5 μl of substrate (2.5 mg/ml in 2-propanol) was added to the cell suspension. The plate was sealed with air permeable foil, and the reaction was incubated on an orbital shaker at 1000 rpm at 28° C. for 22 hours. No conversion of avermectin to 4″-ketoavermectin was detected.

For the cell-free assays, 100 μl cell free extract, 1 μl substrate solution (20 mg/ml) in 2-propanol, 5 μl 100 mM NADPH, 10 μl ferredoxin, 10 μl ferredoxin reductase, and 374 μl potassium phosphate buffer pH 7.0 were added as described in Example III, and the assay was incubated at 30° C. with shaking at 600 rpm for 20 hours. 9.2% +/−0.3% of avermectin was converted to 4″-ketoavermectin.

Thus, expression of the ema1 gene in E. coli resulted in the production of the active Ema1 P450 monooxygenase enzyme which, when purified from the cells, was able to convert avermectin to 4″-ketoavermectin.

### EXAMPLE XVI

### Identification and Cloning of Genes Encoding Ferredoxin Reductases that Support Increased Activity of the P450_{Ema1} Monooxygenase

The electron transport pathway that supports the activity of P450 monooxygenases also includes ferredoxin reductases. These proteins donate electrons to the ferredoxin and, as is the case with ferredoxins and P450 monooxygenases, specific ferredoxin reductases are known to be better electron donors for certain ferredoxins than others.

Accordingly, a number of ferredoxin reductase genes from Streptomyces strains were cloned and were evaluated for their impacts on the biocatalysis reaction. To do this, numerous bacterial ferredoxin reductase (Fre) protein sequences were retrieved from NCBI and aligned with the program Pretty from the GCG package. Two conserved regions, approximately 266 amino acid residues apart, were used to make degenerate oligonucleotides for PCR. The forward primer (CGSCCSCCSCTSWSSAAS (SEQ ID NO: 96; where “S” is C or G; and “W” is A or G)) and the reverse primer (SASSGCSTTSBCCCARTGYTC (SEQ ID NO: 97; where “S” is C or G; “B” is C, G, or T; “R” is A or G; and “Y” is C or T)) were used to amplify 800 bp products from the biocatalytically active Streptomyces strains R-922 and I-1529. These pools of products were cloned into TOPO TA cloning vectors (commercially available from Invitrogen Inc., Carlsbad, Calif.), and 20 clones each from R922 and I-1529 were sequenced according to standard methods (see, e.g., Current Protocols in Molecular Biology, eds. Ausubel et al., John Wiley & Sons, Inc. 2000). Sequencing revealed that 4 unique fre gene fragments were isolated from the strains: three from (fre3,fre12,fre14) and one from I-1529 (fre16). The fre3,fre12,fre14, and fre16 gene fragments were used as probes to identify full-length ferredoxin reductases from genomic clone banks of Streptomyces strains R922 and I-1529. By this approach, the complete coding sequence of each of the 4 different fre genes was cloned and sequenced. The nucleic acid and amino acid sequences are provided as follows:fre3 (SEQ ID NOs: 98 and 99);fre12 (SEQ ID NOs: 100 and 101);fre14 (SEQ ID NOs: 102 and 103); and fre16 (SEQ ID NOs: 104 and 105).

In order to assess the biological activity of each fre gene in relation to the activity of Ema1, each gene was inserted into the ema1/fd233 operon described above, 3′ to the fd233 gene. This resulted in the formation of artificial operons consisting of the ema1,fd233, and individual fre genes that were expressed from the same promoter. The ema1/fd233/fre operons were cloned into the Pseudomonas plasmid pRK290 and introduced into 3 different P. putida strains. These strains were then analysed for Ema1 biocatalysis activity using the whole cell assay and one of the genes, the fre gene fre16 from strain I-1529, was found to increase the activity of P450_{Ema1} monooxygenase by approximately 2-fold. This effect was strain specific, as it was seen only in one of the P. putida strains, ATCC Desposit No. 17453, and not in the other two. In P. putida strain ATCC 17453, the presence of fre gene fre16 resulted in 44% conversion of avermectin to 4″-keto-avermectin, as compared to 23% without this gene. The other fre genes had no impact on the biocatalysis activity in any of the P. putida strains tested.

In a similar approach, each of the ema1/fd233/fre operons were cloned into the Streptomyces plasmids pTUA, pTBBKA, and pEAA, and introduced into S. lividans strain ZX7. In each case there was no impact in S. lividans by any of the fre genes on biocatalysis activity.

### EQUIVALENTS

Those skilled in the art will recognize, or be able to ascertain, using no more than routine experimentation, numerous equivalents to the specific substances and procedures described herein. Such equivalents are considered to be within the scope of this invention.

1. A purified nucleic acid molecule encoding a P450 monooxygenase that regioselectively oxidizes avermectin to 4″-keto-avermectin.

2. The nucleic acid molecule of claim 1, comprising a nucleic acid sequence that is at least 66% identical to SEQ ID NO: 1.

3. The nucleic acid molecule of claim 1, comprising a nucleic acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 94:

4. The nucleic acid molecule of claim 1, wherein the nucleic acid molecule is isolated from a Streptomyces strain.

5. The nucleic acid molecule of claim 4, wherein the Streptomyces strain is selected from the group consisting of Streptomyces lydicus, Streptomyces platensis, Streptomyces chattanoogensis, Streptomyces kasugaensis, and Streptomyces rimosus and Streptomyces albofaciens.

6. The nucleic acid molecule of claim 1 further comprising a nucleic acid sequence encoding a tag which is linked to the P450 monooxygenase via a covalent bond.

7. The nucleic acid molecule of claim 6, wherein the tag is selected from the group consisting of a His tag, a GST tag, an HA tag, a HSV tag, a Myc-tag, and VSV-G-Tag.

8. A purified nucleic acid molecule encoding a polypeptide that exhibits an enzymatic activity of a P450 monooxygenase that regioselectively oxidizes the alcohol at position 4″ of a compound of formula (II), in free form or in salt form 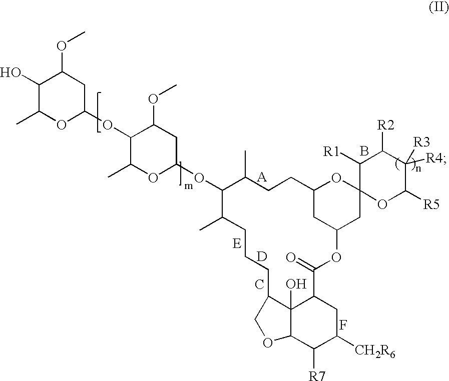
wherein R₁-R₇ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula 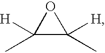
or a single bond and a methylene bridge of the formula 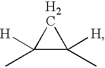
including an E/Z isomer, a mixture of E/Z isomers, and/or a tautomer thereof; in order to produce a compound of the formula (III), in free form or in salt form 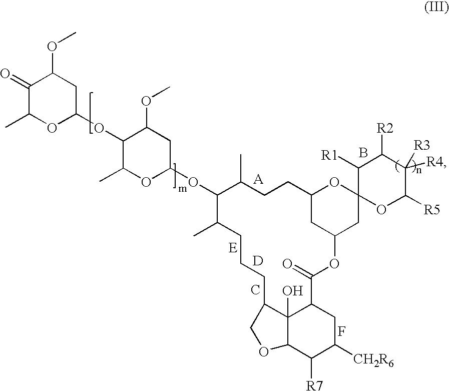
in which R1, R2, R3, R4, R5, R6, R7, m, n, A, B, C, D, E and F have the meanings given for formula (II).

9. A purified P450 monooxygenase that regioselectively oxidizes avermectin to 4″-keto-avermectin.

10. The purified P450 monooxygenase of claim 9, comprising an amino acid sequence that is at least 50% identical to SEQ ID NO: 2.

11. The purified P450 monooxygenase of claim 9, comprising an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, and SEQ ID NO: 95.

12. The purified P450 monooxygenase of claim 9, further comprising a tag.

13. The purified P450 monooxygenase of claim 12, wherein the tag is selected from the group consisting of a His tag, a GST tag, an HA tag, a HSV tag, a Myc-tag, and VSV-G-Tag.

14. A purified polypeptide that exhibits an enzymatic activity of a P450 monooxygenase and that regioselectively oxidizes the alcohol at position 4″ of a compound of formula (II), in free form or in salt form 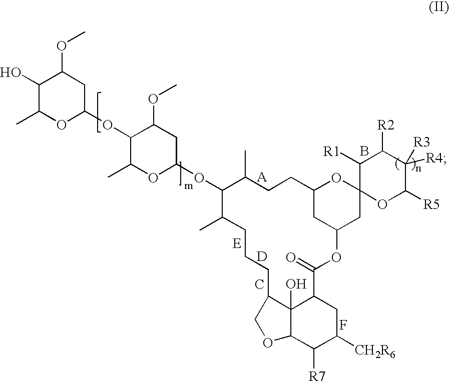
wherein R₁R₇ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula 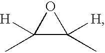
or a single bond and a nethylene bridge of the formula 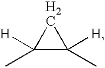
including a E/Z isomer, a mixture of E/Z isomers, and/or a tautomer thereof; in order to produce a compound of the formula (III), in free form or in salt form 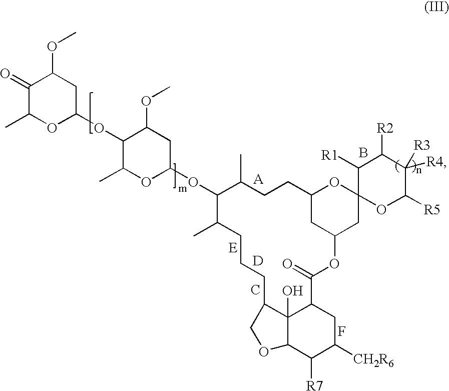
in which R1, R2, R3, R4, R5, R6, R7, m, n, A, B, C, D, E and F have the meanings given for formula (II).

15. A binding agent that specifically binds to the P450 monooxygenase of claim 9,

16. The binding agent of claim 15, wherein the binding agent is an antibody.

17. The binding agent of claim 16, wherein the antibody is selected from the group consisting of a polyclonal antibody and a monoclonal antibody.

18. A family of P450 monooxygenase polypeptides, wherein each member of the family regioselectively oxidizes avermectin to 4″-keto-avermectin.

19. The family of claim 18, wherein each member of the family comprises an amino acid sequence that is at least 50% identical to SEQ ID NO: 2.

20. A family of polypeptides exhibiting an enzymatic activity of a P450 monooxygenase, wherein each member of the family regioselectively oxidizes the alcohol at position 4′ of a compound of formula (II), in free form or in salt form 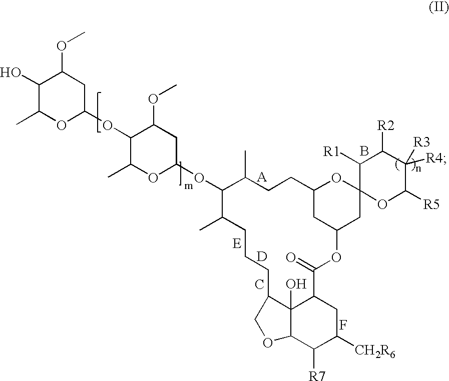
wherein R₁-R₇ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula 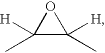
or a single bond and a methylene bridge of the formula 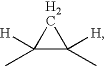
including an E/Z isomer, a mixture of E/Z isomers, and/or a tautomer thereof; in order to produce a compound of the formula (III), in free form or in salt form 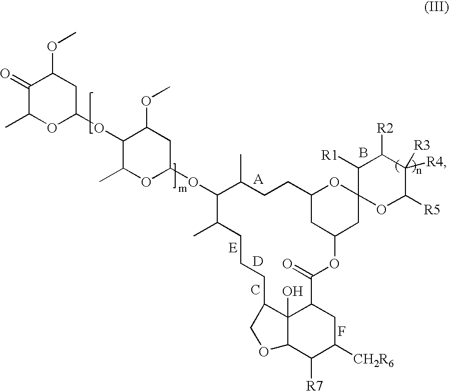
in which R1, R2, R3, R4, R5, R6, R7, m, n, A, B, C, D, E and F have the meanings given for formula (II).

21. A cell genetically engineered to comprise a nucleic acid molecule encoding a P450 monooxygenase that regioselectively oxidizes avermectin to 4″-keto-avermectin.

22. The cell of claim 21, wherein the nucleic acid molecule is positioned for expression in the cell.

23. The cell of claim 21 further comprising a nucleic acid molecule encoding a ferredoxin protein.

24. The cell of claim 21, wherein the cell is a genetically engineered Streptomyces strain cell.

25. The cell of claim 24, wherein the cell is a genetically engineered Streptomyces lividans strain.

26. The cell of claim 25, wherein the cell has NRRL Designation No. B-30478.

27. The cell of claim 21, wherein the cell is a genetically engineered Pseudomonas sstrain.

28. The cell of claim 27, wherein the cell is a genetically engineered Pseudomonas putida strain.

29. The cell of claim 28, wherein the strain has NRRL Designation No.B-30479.

30. The cell of claim 21, wherein the cell is a genetically engineered Escherichia coli strain.

31. The cell of claim 21, further comprising a nucleic acid molecule encoding a ferredoxin reductase protein.

32. The cell of claim 21, further comprising a nucleic acid molecule encoding a ferredoxin protein and a nucleic acid molecule encoding a ferredoxin reductase protein.

33. A purified nucleic acid molecule encoding a ferredoxin, wherein the nucleic acid molecule is isolated from a Streptomyces strain comprising a P450 monooxygenase that regioselectively oxidizes avermectin to 4″-keto-avermectin.

34. The nucleic acid molecule of claim 33, comprising a nucleic acid sequence selected from the group consisting of SEQ ID NO: 35 and SEQ ID NO: 37.

35. A purified ferredoxin protein, wherein the ferredoxin protein is isolated from a Streptomyces strain comprising a P450 monooxygenase that regioselectively oxidizes avermectin to 4″-keto-avermectin.

36. The ferredoxin protein of claim 35, comprising an amino acid sequence selected from the group consisting of SEQ ID NO: 36 and SEQ ID NO: 38.

37. A purified nucleic acid molecule encoding a ferredoxin reductase, wherein the nucleic acid molecule is isolated from a Streptomyces strain comprising a P450 monooxygenase that regioselectively oxidizes avermectin to 4″-keto-avermectin.

38. The nucleic acid molecule of claim 37, comprising a nucleic acid sequence selected from the group consisting of SEQ ID NO: 98, SEQ ID NO: 100, SEQ ID NO: 102, and SEQ ID NO: 104.

39. A purified ferredoxin reductase protein, wherein the ferredoxin reductase protein is isolated from a Streptomyces strain comprising a P450 monooxygenase that regioselectively oxidizes avermectin to 4″-keto-avermectin.

40. The ferredoxin reductase protein of claim 39, comprising an amino acid sequence selected from the group consisting of SEQ ID NO: 99, SEQ ID NO: 101, SEQ ID NO: 103, and SEQ ID NO: 105.

41. A method for making avermectin, comprising adding a P450 monooxygenase that regioselectively oxidizes avermectin to 4″-keto-avermectin to a reaction mixture comprising avermectin and incubating the reaction mixture under conditions that allow the P450 monooxygenase to regioselectively oxidize avermectin to 4″-keto-avermectin.

42. The method of claim 41, wherein the reaction mixture further comprises a ferredoxin protein.

43. The method of claim 41, wherein the reaction mixture further comprises a ferredoxin reductase protein.

44. A method for the preparation a compound of the formula (I), in free form or in salt form 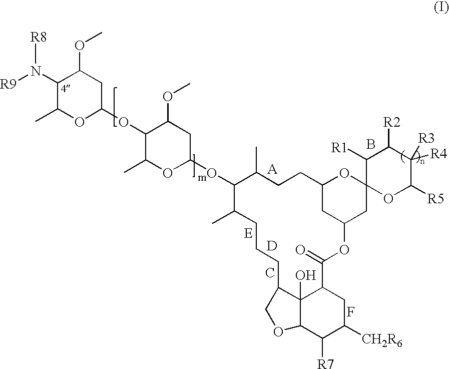
in which R₁-R₉ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula 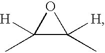
or a single bond and a methylene bridge of the formula 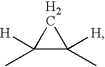
including an E/Z isomer, a mixture of E/Z isomers, and/or a tautomer thereof; which process comprises
1) bringing a compound of the formula (II), in free form or in salt form 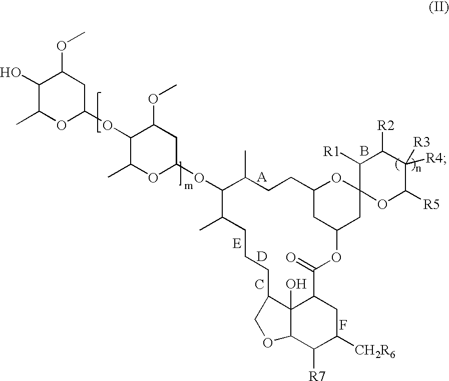
wherein R₁, R₂, R₃, R_{4,} R₅, R₆, and R₇ represent, independently of each other hydrogen or a substituent; and m, n, A, B, C, D, E and F have the same meanings as given for formula (I) above, into contact with a polypeptide that regioselectively oxidizes the alcohol at position 4″ in order to form a compound of the formula (III), in free form or in salt form 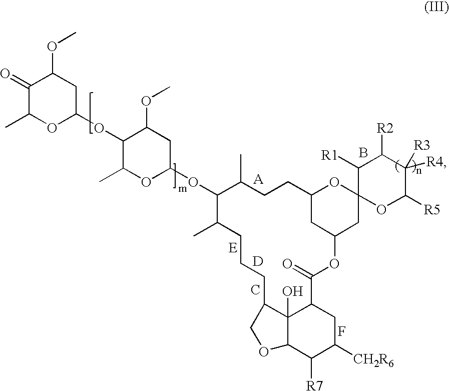
in which R₁, R₂, R₃, R₄, R₅, R₆, and R₇ represent, independently of each other hydrogen or a substituent; and m, n, A, B, C, D, E and F have the meanings given for formula (I); and
2) reacting the compound of the formula (III) with an amine of the formula HN(R₈)R₉, wherein R₈ and R₉ have the same meanings as given for formula (I), in the presence of a reducing agent.

45. The method of claim 44, wherein in the compound of formula (I), n is 1; m is 1; A is a double bond; B is single bond or a double bond; C is a double bond; D is a single bond; E is a double bond; F is a double bond, or a single bond and a epoxy bridge, or a single bond and a methylene bridge; R₁, R₂ and R₃ are H; R₄ is methyl; R₅ is C₁-Cl₁₀-alkyl, C₃-C₈-cycloalkyl or C₂-C₁₀-alkenyl; R₆ is H; R₇ is OH; R₈ and R₉ are independently of each other H; C₁-C₁₀-alkyl or C₁-C₁₀-acyl, or together form —(CH₂)_{q}—, where q is 4, 5 or 6.

46. The method of claim 44, wherein in the compound of formula (I), n is 1; m is 1; A, B, C, E and F are double bonds; D is a single bond; R₁, R₂, and R₃ are H; R₄ is methyl; R₅ is s-butyl or isopropyl; R₆ is H; R₇ is OH; R₈ is methyl; and R₉ is H.

47. A method for the preparation of a compound of the formula (III), in free form or in salt form 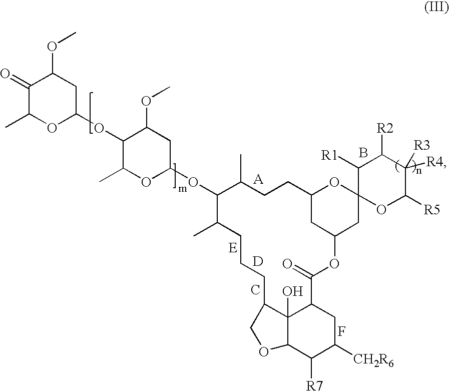
in which R₁-R₉ represent, independently of each other hydrogen or a substituent; m is 0, 1 or 2; n is 0, 1, 2 or 3; and the bonds marked with A, B, C, D, E and F indicate, independently of each other, that two adjacent carbon atoms are connected by a double bond, a single bond, a single bond and a epoxide bridge of the formula 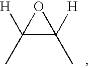
or a single bond and a methylene bridge of the formula 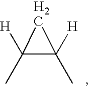
including an E/Z isomer, a mixture of E/Z isomers, and/or a tautomer thereof, which process comprises bringing a compound of the formula (II), in free form or in salt form 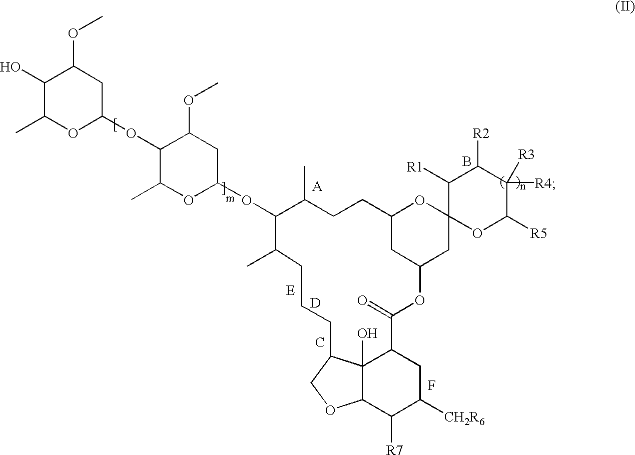
wherein R₁-R₇, m, n, A, B, C, D, E and F have the same meanings as given for formula (III) into contact with a polypeptide that regioselectively oxidizes the alcohol at position 4″, maintaining said contact for a time sufficient for the oxidation reaction to occur and isolating and purifying the compound of formula (III).

48. A formulation for making avermectin comprising a P450 monooxygenase that regioselectively oxidizes avermectin to 4″-keto-avermectin.

49. The formulation of claim 44 further comprising a ferredoxin protein.

50. The formulation of claim 44 further comprising a ferredoxin reductase protein.


SEQUENCE LISTING (105 sequences)

SEQ ID NO: 1 - DNA, 1293 bp, Streptomyces tubercidicus
atgtcggaattaatgaactctccgttcgccgcgcacgtcgggaaacacccgggcgagccgaatgtgatggaccccgccctgatcaccgacccgttcaccggctacggcgcgctgcgtgagcagggcccggtcgtacggggccggttcatggacgactcgcccgtctggctggtgacgcggttcgaggaggtccgccaggtcctgcgcgaccagcggttcgtgaacaatccggcctcgccgtccctgaactacgcgcccgaggacaacccgctgacccggctgatggagatgctgggcctccccgagcacctccgcgtctacctgctcggatcgatcctcaactacgacgcccccgaccacacccggctgcgccgtctggtgtcgcgggcgttcacggcccgcaagatcaccgacctgcggccccgggtcgagcagatcgccgacgcgctgctggcccggctgcccgagcacgccgaggacggcgtcgtcgacctcatccagcacttcgcctaccccctgccgatcaccgtcatctgcgaactggtcggcatacccgaagcggaccgcccgcagtggcgaacgtggggcgccgacctcatctcgatggatccggaccggctcggcgcctcgttcccggcgatgatcgagcacatccatcagatggtccgggaacggcgcgaggcgctcaccgacgacctgctcagcgaactgatccgcacccatgacgacgacggcgggcggctcagcgacgtcgagatggtcaccatgatcctcacgctcgtcctcgccggccacgagaccaccgcccacctcatcagcaacggcacggcggcgctgctcacccaccccgaccagctgcgtctggtcaaggacgatccggccctcctcccccgtgccgtccacgagctgatgcgctggtgcgggccggtgcacatgacccagctgcgctacgccaccgccgacgtcgacctcgccggcacaccgatccgccagggcgatgccgttcaactcatcctggtatcggccaacttcgacccccgtcactacaccgaccccgaccgcctcgatctcacccggcaccccgcgggccacgccgagaaccatgtgggtttcggccatggagcgcactactgcctgggcgccacactcgccaaacaggaaggtgaagtcgccttcggcaaactgctcacgcactacccggacatatcgctgggcatcgccccggaacacctggagcggacaccgctgccgggcaactggcggctgaactcgctgccggtgcggttggggtga

SEQ ID NO: 2 - protein, 430 aa, Streptomyces tubercidicus [sequence omitted]

SEQ ID NO: 3 - DNA, 1293 bp, Streptomyces tubercidicus
atgtcggcattatccagctctccgttcgctgcgcatgtcgggaaacacccgggtgagccgaatgtgatggagccggcgctgctcaccgacccgttcgcgggctacggcgcgctgcgtgagcaggccccggtcgtacggggccggttcgtggacgactcaccggtctggttcgtgacgcgcttcgaggaggtccgccaagtcctgcgcgaccagcggttcgtgaacaatccggccgcgccgcccctggccccatcggccgaggagaacccgctgaccaggctgatggacatgctgggcctccccgagcacctccgcgtctacatgctcgggtcgattctcaactacgacgcccccgaccacacccggctgcgccgtctggtgtcgcgcgcgttcacggcgcggaagatcaccgatctgcgaccgcgtgtcgagcagatcgccgacgagctgctggcccgcctccccgagtacgccgaggacggcgtcgtcgacctcatccagcatttcgcctacccgctgccgatcaccgtcatctgcgagctggtcggcatacccgaagcggaccgcccgcagtggcggaagtggggcgccgacctcatctcgatggacccggaccggctcggcgcaacgttcccggcgatgatcgagcacatccatgagatggtccgggagcggcgcgcggcgctcaccgatgatctgctcagcgagctgatccgtacccatgacgacgatggcggccggctcagcgacgtcgagatggtcaccatgatcctcacgctcgtcctcgccggtcacgagaccaccgcccacctcatcagcaacggcacggcggcgctgctcacccaccccgaccagctgcgcctgctcaaggacgacccggccctgctcccccgggccgtccatgaactgatgcgctggtgcgggccggtgcagatgacgcagctgcgctacgcggccgccgacgtcgacctcgccggtacgcggatccacaagggcgacgccgtacaactcctcctggttgcggcgaacttcgacccccgccactacaccgaccccgaccgtctcgatctgacgcgtcaccccgccggccacgccgagaaccatgtgggtttcggccacggtgcgcattactgcctgggtgccaccctcgccaagcaggagggcgaagtcgcgttcggcaagctgctcgcgcactacccggagatgtccctgggcatcgaaccggaacgtctggagcgattgccgctgcctggcaactggcggctgaattccctgccgttgcggctggggtga

SEQ ID NO: 4 - protein, 430 aa, Streptomyces tubercidicus
MSALSSSPFAAHVGKHPGEPNVMEPALLTDPFAGYGALREQAPVVRGRFVDDSPVWFVTRFEEVRQVLRDQRFVNNPAAPPLAPSAEENPLTRLMDMLGLPEHLRVYMLGSILNYDAPDHTRLRRLVSRAFTARKITDLRPRVEQIADELLARLPEYAEDGVVDLIQHFAYPLPITVICELVGIPEADRPQWRKWGADLISMDPDRLGATFPAMIEHIHEMVRERRAALTDDLLSELIRTHDDDGGRLSDVEMVTMILTLVLAGHETTAHLISNGTAALLTHPDQLRLLKDDPALLPRAVHELMRWCGPVQMTQLRYAAADVDLAGTRIHKGDAVQLLLVAANFDPRHYTDPDRLDLTRHPAGHAENHVGFGHGAHYCLGATLAKQEGEVAFGKLLAHYPEMSLGIEPERLERLPLPGNWRLNSLPLRLG

SEQ ID NO: 5 - DNA, 1413 bp, Streptomyces rimosus
atgaccacatcgcccaccgagtcccgggcggccaccccgcccgactccaccgcctccccctcgaccgcttccgccccggccaccaccccttcggccgccgcctctccggacaccaccgaccgcaccacgctcccctcctacgtcggcctccacccgggcgagccgaacctgatggaaccggagctgctggagaacccgtacaccggctacggcacgctgcgcgagcaggccccgctcgtccgcgcccggttcatcgacgactcgcccatctggctggtgacccgcttcgacgtggtgcgcgaggtgatgcgtgaccagcggttcgtcaacaacccgaccctggtgcccggcatcggcgcggacaaggacccgcgtgcccggctgatcgagctgttcggcatccccgaggacctggccccgtacctcaccgacaacatcctcaccagcgacccgccggaccacacccggctgcgccgcctggtctcccgcgccttcaccgcacgccgtatccaggacctgcggccgcgcgtcgagcggatcaccgacgagctgctggaacggctgccggaccatgccgaggacggcgtcgtcgacctcgtcgagcacttcgcctacccgctgcccatcacggtcatctgcgagctggtcggcatcgacgaggaggatcgggcgctgtggcggcggttcggcgccgacctcgcctcgctgaaccccaagcgcatcggcgccaccatgccggagatgatctcgcacatccacgagctgatcgacgaacggcgcgcggccctgcgggacgacctgctcagcgggctcatccgggcgcaggacgacgacggcggccggctgagcgacgtcgagatggtcaccctggtcctgaccctggtactggccggtcacgagaccaccgcccacctcatcagcaacggcaccctcgccctgctcacccaccccgaccagcggcggctgatcgacgaggacccggcgctgctgccgcgcgcggtccacgagctgatgcgctggtgcgggccgatccaggccacccagcttcggtacgccctggaggacaccgaggtggccggagtccaggtccgccagggcgaggccctgatgttcagcctcgtcgcggccaaccacgacccgcgccactacaccgggccggagcggctcgacctgacgcggcagccggccggccgcgccgaggaccacgtcggcttcggccacggcatgcactactgcctgggtgcctcactcgcccggcaggaggccgaggtggcctacgggaagctgctcacccgctacccggacctggcgctcgccctcaccccggaacagttggaggaccaggaacgcctgcggcagcccggcacctggcgcctgcgacggctgccgctgaggctgcacgcgcagagctga

SEQ ID NO: 6 - protein, 470 aa, Streptomyces rimosus
MTTSPTESRAATPPDSTASPSTASAPATTPSAAASPDTTDRTTLPSYVGLHPGEPNLMEPELLENPYTGYGTLREQAPLVRARFIDDSPIWLVTRFDVVREVMRDQRFVNNPTLVPGIGADKDPRARLIELFGIPEDLAPYLTDNILTSDPPDHTRLRRLVSRAFTARRIQDLRPRVERITDELLERLPDHAEDGVVDLVEHFAYPLPITVICELVGIDEEDRALWRRFGADLASLNPKRIGATMPEMISHIHELIDERRAALRDDLLSGLIRAQDDDGGRLSDVEMVTLVLTLVLAGHETTAHLISNGTLALLTHPDQRRLIDEDPALLPRAVHELMRWCGPIQATQLRYALEDTEVAGVQVRQGEALMFSLVAANHDPRHYTGPERLDLTRQPAGRAEDHVGFGHGMHYCLGASLARQEAEVAYGKLLTRYPDLALALTPEQLEDQERLRQPGTWRLRRLPLRLHAQS

SEQ ID NO: 7 - DNA, 1293 bp, Streptomyces lydicus
atgtcggcatcacccagcaacacgttcaccgagcacgtcggcaagcacccgggcgagccgaacgtgatggatccggcgctgatcggggatccgttcgccggttacggcgcgctgcgcgagcagggcccggtcgtgcgggggcggttcatggacgactcccccgtgtggttcgtgacccgcttcgaggaggtccgcgaggtcctgcgtgacccgcggttccggaacaatccggtctccgcggcgccgggcgcggcccccgaggacaccccgctgtcccggctgatggacatgatgggtttccccgagcacctgcgcgtctatctgctcggctcgatcctcaacaacgacgcccccgaccacacccggctgcgccgcctggtctcccgggccttcaccgcgcggaagatcaccgatctgcggccgcgcgtcacacagatagccgacgagctgctggcccggctgccggagcacgccgaggacggcgtcgtcgacctgatccagcacttcgcctatcccctgccgatcaccgtcatctgcgaactggtcggcatccccgaggaggaccgcccgcagtggcgcacctggggcgccgacctggtctcgctgcagccggaccggatgagccggtccttcccggcgatgatcgaccacatccacgagctgatcgcggcgcggcgccgggcgctcaccgacgatctgctcagcgagctgatccggacccatgacgacgacggcagccggctcagcgacgtcgagatggtcaccatggtcctcaccgtcgtcctggccggccacgagaccaccgcgcacctcatcggcaacggcacggcggccctgctcacccaccccgaccagctgcggctgctcaaggacgacccggcgctgctgccgcgcgcggtgcacgagttgatgcgctggtgcggcccggtgcacatgacccagctgcgctacgccgccgaggacgtcgagctggcgggcgtccggatccgcacgggggacgccgtccagctcatcctggtgtcggcgaaccgcgacccgcgccactacaccgaccccgaccggctggacctgacccggcaccctgccggccacgcggagaaccatgtggggttcggccacggggcgcactactgtctgggcgccacgctcgccaagcaggagggcgaggtcgccctcggcgccctgctcaggcacttccccgagctgtcgctggccgtcgcgccggaggccctggagcgcacaccggtaccgggcagctggcggctgaacgcgctgccgctgcgtctgcgctga

SEQ ID NO: 8 - protein, 430 aa, Streptomyces lydicus
MSASPSNTFTEHVGKHPGEPNVMDPALIGDPFAGYGALREQGPVVRGRFMDDSPVWFVTRFEEVREVLRDPRFRNNPVSAAPGAAPEDTPLSRLMDMMGFPEHLRVYLLGSILNNDAPDHTRLRRLVSRAFTARKITDLRPRVTQIADELLARLPEHAEDGVVDLIQHFAYPLPITVICELVGIPEEDRPQWRTWGADLVSLQPDRMSRSFPAMIDHIHELIAARRRALTDDLLSELIRTHDDDGSRLSDVEMVTMVLTVVLAGHETTAHLIGNGTAALLTHPDQLRLLKDDPALLPRAVHELMRWCGPVHMTQLRYAAEDVELAGVRIRTGDAVQLILVSANRDPRHYTDPDRLDLTRHPAGHAENHVGFGHGAHYCLGATLAKQEGEVALGALLRHFPELSLAVAPEALERTPVPGSWRLNALPLRLR

SEQ ID NO: 9 - DNA, 1299 bp, Streptomyces
atgtcagccttatccagctctccgttcgccgagcacatagggaaacacccgggcgagccgaacgtgatggaaccggctctgatcaacgatccgttcggcggctacggcgcgctgcgcgagcaggggccggttgtgcgtggccggttcatggacgactcgcccgtgtggttcgtgacccgcttcgaggaggtccgccaagtcctgcgcgaccagcggttcgtgaacaatccggcgtcgccgctcctgggcagtcaggtcgaggagatgccgatggtcaagctgctggagcagatgggcctccccgagcaccttcgggtctatctgctcggatcgatcctcaacagtgacgcccccgatcacacccggcttcgccgcctcgtctcgcgggccttcaccgcacgtaagatcaccggtctgcggccgcgcgtcgagcagatcgccgacgagctgctggcccggctccccgagcacgccgaggacggcgtcgtcgacctcatccagcacttcgcctacccgctgccgatcacggtcatctgcgaactggtcggcatacccgaagccgatcgcccgcaatggcgcgcatggggcgccgacctcgtgtcactggagccggacaagctcagcacgtcgttcccggcgatgatcgaccacacccatgaactgatccgccaacggcgcggcgcgctcaccgacgatctgctcagcgagctgatccgtgcccatgacgacgacggcagccggctcagcgacgtcgagatggtcaccatggtgttcgctctcgtcttcgccggtcacgagaccaccgcccacctcataggcaacggcacggcggcgctgctcacccaccccgaccagctgcgcctgctcaaggacgacccggccctgctcccgcgtgccgtccatgagctgatgcgctggtgcgggccggtgcacatgacccagttgcgttacgcctccgaggacatcgacctcgccggtacgccgatccggaagggcgacgccgtccaactcatcctggtatcggcgaacttcgacccccgccactacagcgaccccgatcgcctcgacctgacccgtcaccccgcaggccacgccgagaaccacgtgggcttcggccacgggatgcactactgcttgggcgccgcgctcgccaggcaggaaggcgaagtggcgttcggcaaactgctcgcgcactacccggacgtagcgctgggcgtcgaaccggaagccctggagcgggtgccgatgcccggcagttggcggctgaattccttgccgctgcggttggcgaagcgctaa

SEQ ID NO: 10 - protein, 432 aa, Streptomyces
MSALSSSPFAEHIGKHPGEPNVMEPALINDPFGGYGALREQGPVVRGRFMDDSPVWFVTRFEEVRQVLRDQRFVNNPASPLLGSQVEEMPMVKLLEQMGLPEHLRVYLLGSILNSDAPDHTRLRRLVSRAFTARKITGLRPRVEQIADELLARLPEHAEDGVVDLIQHFAYPLPITVICELVGIPEADRPQWRAWGADLVSLEPDKLSTSFPAMIDHTHELIRQRRGALTDDLLSELIRAHDDDGSRLSDVEMVTMVFALVFAGHETTAHLIGNGTAALLTHPDQLRLLKDDPALLPRAVHELMRWCGPVHMTQLRYASEDIDLAGTPIRKGDAVQLILVSANFDPRHYSDPDRLDLTRHPAGHAENHVGFGHGMHYCLGAALARQEGEVAFGKLLAHYPDVALGVEPEALERVPMPGSWRLNSLPLRLAKR

SEQ ID NO: 11 - DNA, 1293 bp, Streptomyces chattanoogenesis
atgtcggcatcacccagcaacacgttcaccgagcacgtcggcaagcacccgggcgagccgaacgtgatggatccggcgctgatcggtgatccgttcgccggttacggcgcgctgcgcgagcagggcccggtcgtgcgggggcggttcatggacgactcccccgtgtggttcgtgacccgcttcgaggaggtccgcgaggtcctgcgtgacccgcggttccggaacaatccggtctccgcggcgccgggcgcggcccccgaggacaccccgctgtcccggctgatggacatgatgggtttccccgagcacctgcgcgtctatctgctcggctcgatcctcaacaacgacgcccccgaccacacccggctgcgccgcctggtctcccgggccttcaccgcgcggaagatcaccgatctgcggccgcgcgtcacacagatagccgacgagctgctggcccggctgccggagcacgccgaggacggcgtcgtcgacctgatccagcacttcgcctatcccctgccgatcaccgtcatctgcgaactggtcggcatccccgaggaggaccgcccgcagtggcgcacctggggcgccgacctggtctcgctgcagccggaccggatgagccggtccttcccggcgatgatcgaccacatccacgagctgatcgcggcgcggcgccgggcgctcaccgacgacctgctcagcgagctgatccggacccatgacgacgacggcagcaggctcagcgacgtcgagatggtcaccatggtcctcaccgtcgtcctggccggccacgagaccaccgcgcacctcatcggcaacggcacggcggccctgctcacccaccccgaccagctgcggctgctcaaggacgacccggcactgctgccgcgcgcggtgcacgagttgatgcgctggtgcggcccggtgcacatgacccagctgcgctacgccgccgaggacgtcgagctggcgggcgtccggatccgcacgggggacgccgtccagctcatcctggtgtcggcgaaccgcgacccgcgccactacaccgaccccgaccgtctggacctgacccggcaccccgccggtcacgcggagaaccatgtggggttcggccacggggcgcactactgtctgggcgccacgctcgccaagcaggagggcgaggtcgccctcggcgccctgctcaggcacttccccgagctgtcgctggccgtcgcgccggacgccctggagcgcacaccggtaccgggcagctggcggctgaacgcgctgccgctgcgtctgggctga

SEQ ID NO: 12 - protein, 430 aa, Streptomyces chattanoogenesis
MSASPSNTFTEHVGKHPGEPNVMDPALIGDPFAGYGALREQGPVVRGRFMDDSPVWFVTRFEEVREVLRDPRFRNNPVSAAPGAAPEDTPLSRLMDMMGFPEHLRVYLLGSILNNDAPDHTRLRRLVSRAFTARKITDLRPRVTQIADELLARLPEHAEDGVVDLIQHFAYPLPITVICELVGIPEEDRPQWRTWGADLVSLQPDRMSRSFPAMIDHIHELIAARRRALTDDLLSELIRTHDDDGSRLSDVEMVTMVLTVVLAGHETTAHLIGNGTAALLTHPDQLRLLKDDPALLPRAVHELMRWCGPVHMTQLRYAAEDVELAGVRIRTGDAVQLILVSANRDPRHYTDPDRLDLTRHPAGHAENHVGFGHGAHYCLGATLAKQEGEVALGALLRHFPELSLAVAPDALERTPVPGSWRLNALPLRLG

SEQ ID NO: 13 - DNA, 1290 bp, Streptomyces
atgaccgaattagcggactcccccttcagcgagcacgtcggcaaacaccccggcgagccgaacgtgatggaaccggccctgctcaccgatccgttcaccggctacggcgaactgcgcgaacagggcccggtggtccgcggccggttcgcggacgacacccccgtgtggttcatcacccgcttcgaggaggcccgcgaggtgctgcgcgaccaccggttcgccaatgcccccgccttcgcggcgggaggtggaagcggtgacacaccctccaaccggctgatggaaatcatgggcctgcccgagcactaccgggtgtacctcgccaacaccatcctcaccatggacgcccccgaccacacccggatccggcgattggtctcccgggcattcaccgcccgtaagatcaccgatctgcgaccccgggtggaggacatcgcggacgatctgctgaggcggctgcccgagcacgccgaggacggcgtcgtcgacctcatcaagcactacgcctatccgctgcccataacggtcatctgcgaactggtgggaattccggaggaagaccgactgcagtggcgggattgggggtccgcgttcgtctccctgcaaccggatcggctcagcaaagcgttcccggcgatgatcgaacacattcacgcgctgatccgcgaacggcgcgcggcgctcaccgacgatctgctcagcgaactgatccgggtccatgacgacgacggcggccgactcagcgacgtcgaaatggtcacgatggtcctgaccctcgttctcgccggtcatgagaccaccgcccatctcatcggcaacggcactgccgcgcttctcacccaccccgaccagctgcacctgctgaaatccgatccggagctgctcccacgcgccgtgcacgagctgatgcgctggtgcggaccggtgcagatgacgcagttgcggtacgccaccgaggacgtcgaggtggccggggtgcaggtcaagcagggcgaagcggtgctggccatgctggtcgcggcgaaccacgacccccgccacttcgccgaccccgcccggctcgacctcacccgccagccggcgggccgggccgagaaccacgtcggtttcggccacggcatgcactactgcctgggcgccagcctggcccgccaggagggcgaggtcgccttcgggaacctgctcgcgcactacccggacgtgtcgctggcggtggaaccggacgccctccagcgggtcccgctgccgggcaactggcggctggccgcactgccggtccggctgcgctga

SEQ ID NO: 14 - protein, 429 aa, Streptomyces
MTELADSPFSEHVGKHPGEPNVMEPALLTDPFTGYGELREQGPVVRGRFADDTPVWFITRFEEAREVLRDHRFANAPAFAAGGGSGDTPSNRLMEIMGLPEHYRVYLANTILTMDAPDHTRIRRLVSRAFTARKITDLRPRVEDIADDLLRRLPEHAEDGVVDLIKHYAYPLPITVICELVGIPEEDRLQWRDWGSAFVSLQPDRLSKAFPAMIEHIHALIRERRAALTDDLLSELIRVHDDDGGRLSDVEMVTMVLTLVLAGHETTAHLIGNGTAALLTHPDQLHLLKSDPELLPRAVHELMRWCGPVQMTQLRYATEDVEVAGVQVKQGEAVLAMLVAANHDPRHFADPARLDLTRQPAGRAENHVGFGHGMHYCLGASLARQEGEVAFGNLLAHYPDVSLAVEPDALQRVPLPGNWRLAALPVRLR

SEQ ID NO: 15 - DNA, 1428 bp, Streptomyces albofaciens
atgaccacatcgcccaccgagtcccgggcggccaccccgcccgactccaccgcctccccctcgaccgctgccgccccggccaccaccccttcggccgccgcctctccggacaccacctctcccgccaccaccgaccgcaccacgctcccctcctacgtcggcctccacccgggcgagccgaacctgatggaaccggagctgctggacaacccgtacaccggctacggcacgctgcgcgagcaggcgccgctcgtccgcgcccggttcatcgacgactcgcccatctggctggtgacccgcttcgacgtggtgcgcgaggtgatgcgcgaccagcggttcgtcaacaacccgaccctggtgcccggcatcggtgcggaccaggacccgcgcgcccggctgatcgagctgttcggcatccccgaggacctggccccgtacctcaccgacaccatcctcaccagcgacccgccggaccacacccggctgcgccgcctggtctcccgtgccttcaccgcacgccgtatccaggacctgcggccgcgcgtcgagcggatcaccgacgagctgctggcgcggctgccggaccatgccgaggacggcgtcgtcgacctcgtcgagcacttcgcctacccgctgcccatcacggtcatctgcgaactggtcggcatcgacgaggaggaccgggcgctgtggcggcggttcggcgccgacctcgcctcgctgaaccccaagcgcatcggcgccaccatgccggagatgatcgcgcacatccacgaggtgatcgacgagcggcgtgcggacctgcgggacgacctgctcagcgggctcatccgggcgcaggacgacgacggcggccggctgagcgacgtcgagatggtcacgctggtgctgaccctggtgctggccggtcacgagaccaccgcccacctcatcagcaacggcaccctcgccctgctcacccaccccgaccagcggcggctgatcgacgaggacccggcgctgctgccgcgcgcggtccacgagctgatgcgctggtgcgggccgatccaggccacccagctgcggtacgccatggaggacaccgaggtggccggtgtccaggtccgccagggcgaggccctgatgttcagcctcgtcgcggccaaccacgacccgcgccactacaccggcccggagcggctcgacctgacgcggcagccggccggccgcgccgaggaccacgtcggcttcgggcacgggatgcactactgcctgggtgcctcactggcccggcaggaggccgaggtggcgtacggcaagctgctcacccgctacccggacctggcgctcgcgctcaccccggaacagctggaggaccaggaacgcctgcggcagcccggcacctggcgcctgcgacggctgccgctgaggttgcacgcggagagctga

SEQ ID NO: 16 - protein, 475 aa, Streptomyces albofaciens
MTTSPTESRAATPPDSTASPSTAAAPATTPSAAASPDTTSPATTDRTTLPSYVGLHPGEPNLMEPELLDNPYTGYGTLREQAPLVRARFIDDSPIWLVTRFDVVREVMRDQRFVNNPTLVPGIGADQDPRARLIELFGIPEDLAPYLTDTILTSDPPDHTRLRRLVSRAFTARRIQDLRPRVERITDELLARLPDHAEDGVVDLVEHFAYPLPITVICELVGIDEEDRALWRRFGADLASLNPKRIGATMPEMIAHIHEVIDERRADLRDDLLSGLIRAQDDDGGRLSDVEMVTLVLTLVLAGHETTAHLISNGTLALLTHPDQRRLIDEDPALLPRAVHELMRWCGPIQATQLRYAMEDTEVAGVQVRQGEALMFSLVAANHDPRHYTGPERLDLTRQPAGRAEDHVGFGHGMHYCLGASLARQEAEVAYGKLLTRYPDLALALTPEQLEDQERLRQPGTWRLRRLPLRLHAES

SEQ ID NO: 17 - DNA, 1293 bp, Streptomyces
atgtcggcattacccacctcaccgttcgctgcacacgtcgggaaacacccgggcgagccgaatgtgatggacccggcactgatcaccgacccgttcaccggctacggcgcgctgcgcgagcagggcccggtcgtccgcggccgcttcgtggacgactcacccgtctggctggtgacgcgattcgaggaggtccgccaagtcctgcgcgaccagcggttcgtgaacaacccggcggcgccctccctgggccacgcggccgaggacaacccgctcaccaggctgatggacatgctgggcctccccgagcacctccgcccctacctcctcggatcgattctcaattacgacgcccccgaccacacccggctgcgccgcctggtgtcgcgggccttcaccgcccgcaagatcaccgacctgcggccgcgggtcgagcagatcgccgacgccctgctggcccggctgcccgagcacgccgaggacggcgtcgtcgatctcatccggcacttcgcctacccgctgccgatcaccgtcatctgcgaactggtcggcatacccgaagcggaccgcccgcagtggcggacgtggggcgccgacctcgtctcgatggagccggaccggctcaccgcctcgttcccgccgatgatcgagcacatccaccggatggtccgggagcggcgcggcgcgctcaccggcgatctgctcagcgagctgatccgtgcccatgacgacgacggcggccggctcagcgacgtcgagatggtcaccttgatcctcacgctcgtcctcgccggtcacgagaccaccgctcacctcatcagcaacggcacggcggcgctgctcacccaccccgaccaactgcgcctgctccaggacgacccggccctgctcccccgtgccgtccacgagctgatgcgctggtgcgggccggtgcagatgacccagctgcgttacgccgccgccgacgtcgacctggccggcaccacgatccaccggggcgacgccgtccaactcatcctggtgtcggcgaacttcgacccccgccactacaccgaccccgaccgcctcgatctgacccgccaccccgcgggacatgcggagaaccatgtgggtttcggccatggggcgcactactgcctgggcgccacactcgccaagcaggagggcgaagtcgccttcggcaaactgctcgcgcactacccggagatggcgttgggcgtcgcaccggagcgcctggagcggacgcccctgccgggcaactggcggctgaacgcgctgccggtgcggttggggtga

SEQ ID NO: 18 - protein, 430 aa, Streptomyces
MSALPTSPFAAHVGKHPGEPNVMDPALITDPFTGYGALREQGPVVRGRFVDDSPVWLVTRFEEVRQVLRDQRFVNNPAAPSLGHAAEDNPLTRLMDMLGLPEHLRPYLLGSILNYDAPDHTRLRRLVSRAFTARKITDLRPRVEQIADALLARLPEHAEDGVVDLIRHFAYPLPITVICELVGIPEADRPQWRTWGADLVSMEPDRLTASFPPMIEHIHRMVRERRGALTGDLLSELIRAHDDDGGRLSDVEMVTLILTLVLAGHETTAHLISNGTAALLTHPDQLRLLQDDPALLPRAVHELMRWCGPVQMTQLRYAAADVDLAGTTIHRGDAVQLILVSANFDPRHYTDPDRLDLTRHPAGHAENHVGFGHGAHYCLGATLAKQEGEVAFGKLLAHYPEMALGVAPERLERTPLPGNWRLNALPVRLG

SEQ ID NO: 19 - DNA, 1293 bp, Streptomyces kasugaensis; identical to SEQ ID NO: 7

SEQ ID NO: 20 - protein, 430 aa, Streptomyces kasugaensis; identical to SEQ ID NO: 8

SEQ ID NO: 21 - DNA, 1428 bp, Streptomyces
atgaccacatcgcccaccgagtcccgggcggccaccccgaccggctccaccgcctccccctcgaccgcttccgccccggccaccaccccttcggccgccacctcttcggacaccacctatcccgccaccaccgaccgcaccacgctcccctcctacgtcggcctccacccgggcgagccgaacctgatggaaccggagctgctggacaacccgtacaccggctacggcacgctgcgcgagcaggccccgctcgtccgtgcccggttcatcgacgactcgcccatctggctggtgacccgcttcgacgtggtgcgcgaggtgatgcgcgaccagcggttcgtcaacaacccgaccctggtgcccggcatcggtgcggacaaggacccgcgcgcccggctgatcgagctgttcggcatccccgaggacctgaccccgtacctcgccgacaccatcctcaccagcgacccgccggaccacacccggctgcgccgcctggtctcccgtgccttcaccgcgcgccgcatccaggacctgcggccgcgcgtcgagcagatcaccgacgcgctgctggagcgactgccggaccatgccgaggacggcgtcgtcgacctcgtcgagcacttcgcctacccgctgcccatcacggtcatctgcgagctggtcggcatcgacgaggaggaccggacgctgtggcggcggttcggcgccgacctcgcctcactgaaccccaagcgcatcggcgccaccatgccggagatgatcgcgcacatccacgaggtgatcgacgagcggcgcgcggccctgcgggacgacctgctcagcgggctcatccgggcgcaggacgacgacggcggccggctgagcgacgtcgagatggtcaccctggtcctgaccctggtgctggccggtcacgagaccaccgcccacctcatcagcaacggcaccctcgccctgctcacccaccccgaccagcggcggctgatcgacgaggacccggcactgctgccgcgcgcggtccacgagctgatgcgctggtgcgggccgatccaggccacccagctgcggtacgccatggaggacaccgaggtcgccggtgtccaggtccgccagggcgaggccctgatgttcagcctcgtcgcggccaaccacgacccgcgccactacaccgggccggagcggctcgacctgacgcggcagccggccggccgcgccgaggaccacgtcggcttcgggcacgggatgcactactgcctgggtgcctcactcgcccggcaggaggccgaggtggcctacgggaagctgctcacccgctacccggacctggagctcgctctcacaccggaacagctggaggaccaggaacgcctgcggcagcccggcacctggcgcctgcggcggctgccgctgaagctgcacgcgcggagctga

SEQ ID NO: 22 - protein, 475 aa, Streptomyces
MTTSPTESRAATPTGSTASPSTASAPATTPSAATSSDTTYPATTDRTTLPSYVGLHPGEPNLMEPELLDNPYTGYGTLREQAPLVRARFIDDSPIWLVTRFDVVREVMRDQRFVNNPTLVPGIGADKDPRARLIELFGIPEDLTPYLADTILTSDPPDHTRLRRLVSRAFTARRIQDLRPRVEQITDALLERLPDHAEDGVVDLVEHFAYPLPITVICELVGIDEEDRTLWRRFGADLASLNPKRIGATMPEMIAHIHEVIDERRAALRDDLLSGLIRAQDDDGGRLSDVEMVTLVLTLVLAGHETTAHLISNGTLALLTHPDQRRLIDEDPALLPRAVHELMRWCGPIQATQLRYAMEDTEVAGVQVRQGEALMFSLVAANHDPRHYTGPERLDLTRQPAGRAEDHVGFGHGMHYCLGASLARQEAEVAYGKLLTRYPDLELALTPEQLEDQERLRQPGTWRLRRLPLKLHARS

SEQ ID NO: 23 - DNA, 1293 bp, Streptomyces tubercidicus
atgtcggcattatccaactccccgctcgccgcacatgtcgggaaacaccctggcgagccgaatgtgatggacccggcgctgatcaccgacccgttcggcggctacggcgcactgcgcgagcaaggcccggtcgtacggggccggttcatggacgactcgcccgtctggctggtgacgcgcttcgaagaggtccgccaagtcctgcgcgatcagcggttcgtgaacaacccggccgcaccgtccctgggacgctcgatcgacgaaagccccgcggtcagacttttggaaatgttggggttgcccgaccatttccggccgtatctgctcgggtcgatcctcaactacgacgcacccgaccacacccggctccgccgactggtctcgcgcgccttcacggcacgcaagatcaccgacctgcggccgcgggtcgagcagatcaccgacgacctgctgacccggcttcccgagcacgccgaggacggtgtggtcgacctcatccagcacttcgcctaccccctgccgatcaccgtgatctgcgaactggtcggcatcgccgaagcggaccgcccgcaatggcggaagtggggagccgatctcgtctcgctggagccggggcggctgagcaccgcgttcccggcgatggtcgagcacatccatgagctgatccgcgagcggcgcggcgcgctcaccgacgatctgctcagcgagctgatccgcacccatgacgacgacggcggccggctcagcgacatcgagatggtcaccatgatcctcacgatcgtcctggccggccacgagaccaccgcccacctcataggcaacggcacggcggcgctgctcacccaccccgaccagctgcgcctactcaaggacgatccggcgctgctgccgcgcgccgtccacgagctgatgcgctggtgcgggccggtgcacatgacccagctgcggttcgcgtccgaggacgtcgaggtcgccgggacaccgatccacaagggcgacgccgtacaactcatcctggtatcggcgaacttcgacccccgccactacaccgaccccgaccgtctcgacctgacccgccaccccgccggccacgccgagaaccatgtgggcttcggccacggaatgcactactgcctgggtgccaccctcgccaaacaggaaggcgaagtcgccttctcccgcctcttcacgcactacccggaactgtccctgggcgtcgcggcggaccagctggcgcggacacaggtacccggcagctggcggctggacaccctgccgctgcgactggggtga

SEQ ID NO: 24 - protein, 430 aa, Streptomyces tubercidicus
MSALSNSPLAAHVGKHPGEPNVMDPALITDPFGGYGALREQGPVVRGRFMDDSPVWLVTRFEEVRQVLRDQRFVNNPAAPSLGRSIDESPAVRLLEMLGLPDHFRPYLLGSILNYDAPDHTRLRRLVSRAFTARKITDLRPRVEQITDDLLTRLPEHAEDGVVDLIQHFAYPLPITVICELVGIAEADRPQWRKWGADLVSLEPGRLSTAFPAMVEHIHELIRERRGALTDDLLSELIRTHDDDGGRLSDIEMVTMILTIVLAGHETTAHLIGNGTAALLTHPDQLRLLKDDPALLPRAVHELMRWCGPVHMTQLRFASEDVEVAGTPIHKGDAVQLILVSANFDPRHYTDPDRLDLTRHPAGHAENHVGFGHGMHYCLGATLAKQEGEVAFSRLFTHYPELSLGVAADQLARTQVPGSWRLDTLPLRLG

SEQ ID NO: 25 - DNA, 1293 bp, Streptomyces
atgtcggcattatccagctcaccgttcgccgcgcatgtcgggaaacacccgggcgagccgaatgtgatggacccggcgctgatcgccgatccgttcggtggttatggcgcactgcgtgagcaagggccggtcgtacggggccggttcatggacgactcacccgtctggctcgtgacgcgcttcgaggaagtccgccaagtcctgcgcgaccagcggttcctgaacgatccgacggccccctccctggggcgctcattcgacgacagccccacggccaggctgctggagatgatgggactgcccgagcatttccggccgtatctgctcggttcgattctgaacaacgacgcccccgaccacacccggctgcgccgtctggtgtcgcgcgccttcacggcacgcaagatcaccgacctgcggccgcgggtcgagcagatcgccgacgagctgctgacccggcttcccgagtacgccgaggacggcgtggtcgacctcatcaagcacttcgcctaccccctgccgatcgccgtcatctgcgaactggtcggcatagccgaagcggatcgtccgcagtggcggaagtggggtgccgacctcgtctcgctgcagccggaccggctcagcacctcgttcccggcgatgatcgagcacatccatgagctgatccgcgagcggcgcggggcgctcacggacgatctgctcagcgagctgatccgtgcccatgacgacgacggcggccggctcagcgacgtcgagatggtcaccatgatcctcacggtggtgctcgccggccacgagaccaccgcgcacctcataggcaacggcacggcggcgctgctcacccaccccgaccagctgcggctgctcagggacgacccggctctgtttccccgtgccgtccacgagctgttgcgctggtgcgggccggtccacatgacccagatgcggtttgcgtccgaggatgtcgacatcgccgggacgaagatccgtaagggcgacgccgtacaactgatcctggtatcggccaacttcgacccccgccactacaccgaccccgaacgtctcgacctgacccgtcaccccgccggccacgccgagaaccatgtgggcttcggccacgggatgcactactgcctgggcgccaccctcgccaaacaggagggcgaagtcgcgttcgagaagctcttcgcgcactacccggaggtgtcgctgggcgtcgcaccggaacaactggaaaggacaccactgcccggcagctggcggctcgattccctgccgctgcggttgcggtaa

SEQ ID NO: 26 - protein, 430 aa, Streptomyces
MSALSSSPFAAHVGKHPGEPNVMDPALIADPFGGYGALREQGPVVRGRFMDDSPVWLVTRFEEVRQVLRDQRFLNDPTAPSLGRSFDDSPTARLLEMMGLPEHFRPYLLGSILNNDAPDHTRLRRLVSRAFTARKITDLRPRVEQIADELLTRLPEYAEDGVVDLIKHFAYPLPIAVICELVGIAEADRPQWRKWGADLVSLQPDRLSTSFPAMIEHIHELIRERRGALTDDLLSELIRAHDDDGGRLSDVEMVTMILTVVLAGHETTAHLIGNGTAALLTHPDQLRLLRDDPALFPRAVHELLRWCGPVHMTQMRFASEDVDIAGTKIRKGDAVQLILVSANFDPRHYTDPERLDLTRHPAGHAENHVGFGHGMHYCLGATLAKQEGEVAFEKLFAHYPEVSLGVAPEQLERTPLPGSWRLDSLPLRLR

SEQ ID NO: 27 - DNA, 1293 bp, Streptomyces
atgtcggcattatccagctctccgttcgctgcgcatgtcgggaaacacccgggtgagccgaatgtgatggagccggcgctgctcaccgacccgttcgcgggctacggcgcgctgcgtgagcaggccccggtcgtacggggccggttcgtggacgactcaccggtctggttcgtgacgcgcttcgaggaggtccgccaagtcctgcgcgaccagcggttcgtgaacaatccggccgcgccgcccctggccccatcggccgaggagaacccgctgaccaggctgatggacatgctgggcctccccgagcacctccgcgtctacatgctcgggtcgattctcaactacgacgcccccgaccacacccggctgcgccgtctggtgtcgcgcgcgttcacggcgcggaagatcaccgatctgcgaccgcgtgtcgagcagatcgccgacgagctgctggcccgcctccccgagtacgccgaggacggcgtcgtcgacctcatccagcatttcgcctacccgctgccgatcaccgtcatctgcgagctggtcggcatacccgaagcggaccgcccgcagtggcggaagtggggcgccgacctcatctcgatggacccggaccggctcggcgcaacgttcccggcgatgatcgagcacatccatgagatggtccgggagcggcgcgcggcgctcaccgatgatctgctcagcgagctgatccgtacccatgacgacgatggcggccggctcagcgacgtcgagatggtcaccatgatcctcacgctcgtcctcgccggtcacgagaccaccgcccacctcatcagcaacggcacggcggcgctgctcacccaccccgaccagctgcgcctgctcaaggacgacccggccctgctcccccgggccgtccatgagctgatgcgctggtgcgggccggtgcagatgacgcagctgcgctacgcggccgccgacgtcgacctcgccggtacgcggatccacaagggcgacgccgtacaactcctcctggttgcggcgaacttcgacccccgccactacaccgaccccgaccgtctcgatctgacgcgtcaccccgccggccacgccgagaaccatgtgggtttcggccacggtgcgcattactgcctgggtgccaccctcgccaagcaggagggcgaagtcgcgttcggcaagctgctcgcgcactacccggagatgtccctgggcatcgaaccggaacgtctggagcgattgccgctgcctggcaactggcggctgaattccctgccgttgcggctggggtga

SEQ ID NO: 28 - protein, 430 aa, Streptomyces; identical to SEQ ID NO: 4

SEQ ID NO: 29 - DNA, 1293 bp, Streptomyces lydicus
atgtcggcattacccagcaacacgttcaccgagcacgtcggcaagcacccgggcgaaccgaacgtgatggatccggcgctgatcggtgatccgttcgccggttacggcgcgctgcgcgagcagggcccggtcgtgcgggggcggttcgtggacgactcccccgtgtggttcgtgacccgcttcgaggaggtccgcgaggtcctgcgggaccagcggttccggaacaatccggtctcctcggcgccggacgcggaccccgaggacaccccgctgtcccggctgatggacatgatgggtttccccgagcacctgcgcgtctatctgctcggctcgatcctcaacaacgacgcccccgaccacacccggctgcgccgcctggtctcccgggccttcaccgcgcggaagatcaccgatctgcggccgcgcgtcgcacagatagccgacgagctgctggcccggctgccggagcacgccgaggacggcgtcgtcgacctgatccagcacttcgcctatcccctgccgatcaccgtcatctgcgaactggtcggcatccccgaggaggaccgcccgcagtggcgcacctggggcgccgacctggtctcgctgcagccggaccggatgagccggtccttcccggcgatgatcgaccacatccacgagctgatcgcggcgcggcgccgggcgctcaccgacgacctgctcagcgagctgatccgaacccatgacgacgacggcagccggctcagcgacgtcgagatggtcaccatggtcctcaccgtcgtcctggccggccacgagaccaccgcgcacctcatcggcaacggcacggcggccctgctcacccaccccgaccagctgcggctgctcaaggacgacccggcgctgctgccgcgcgcggtgcacgagttgatgcgctggtgcggcccggtgcacatgacccagctgcgctacgccgccgaggacgtcgagctggcgggcgtccggatccgcaagggggacgccgtccagctcatcctggtgtcggcgaaccgcgatccgcgccactacaccgaacccgaccgtctggacctgacccggcaccccgccggccacgccgagaaccatgtggggttcggccacggggcgcactactgtctgggcgccacgctcgccaagcaggagggcgaggtcgccctcggcgccctgctcaggcacttccccgagctgtcgctggccgtcgcgccggacgccctggagcgcacaccggtaccgggcagctggcggctgaatgcgctgccgctgcgtctgcgctga

SEQ ID NO: 30 - protein, 430 aa, Streptomyces lydicus
MSALPSNTFTEHVGKHPGEPNVMDPALIGDPFAGYGALREQGPVVRGRFVDDSPVWFVTRFEEVREVLRDQRFRNNPVSSAPDADPEDTPLSRLMDMMGFPEHLRVYLLGSILNNDAPDHTRLRRLVSRAFTARKITDLRPRVAQIADELLARLPEHAEDGVVDLIQHFAYPLPITVICELVGIPEEDRPQWRTWGADLVSLQPDRMSRSFPAMIDHIHELIAARRRALTDDLLSELIRTHDDDGSRLSDVEMVTMVLTVVLAGHETTAHLIGNGTAALLTHPDQLRLLKDDPALLPRAVHELMRWCGPVHMTQLRYAAEDVELAGVRIRKGDAVQLILVSANRDPRHYTEPDRLDLTRHPAGHAENHVGFGHGAHYCLGATLAKQEGEVALGALLRHFPELSLAVAPDALERTPVPGSWRLNALPLRLR

SEQ ID NO: 31 - DNA, 1293 bp, Streptomyces lydicus
atgtcggcatcgaccagctctcccctcagcgcccacgtcggcaagcacccgggcgaaccccatgtgatggatccggcgctgatcagcgatccgttcggcggctacggtgccctgcgcgagcagggaccggtcgtccgcggacggttcttcgacgactcgcccttgtggttagtgacccgcttcgaggaagtccgccaggtcctgcgcgaccagcggttcgtgaacaaccccgccgacccggcgctcggcgtcgcgccggaggactccccgcagctgcgcgcgctggcgatgctgggcatccccgagcacctgcacggctatctgctcaactcgatcctcaactacgacgcccccgaccacacccggctgcgccgcctggtctcccgcgccttcaccgcccgcaagatcaccgatcttcggccgcgggtggcgcagataaccgccgagctgctggaccgactcccggagcacgccgaggacggcgtggtcgacctgatcgagcacttcgcctacccgctgccgatcacggtgatctgcgaacttgtcggcatcgccgcggaggaccggccccagtggcgttcctggggcgccgacctggtctcggtggaccccgaccggctcggccggaccttcccggcgatgatcgaccacatccacgcgctgatcggccagcggcgggccgcgctcaccgacgacctgctcagcgagctgatccggacccatgacgacgacggcagccggctcagcgacgtcgagatggtcaccctggtcctcaccctcgtgctggccggccacgagaccaccgcacacctcatcggcaacggcaccgcggccctgctcacccaccccgaccagctgcggctgctcaaggacgacccggcgctgctgccgcgcgccgtccacgagctgatgcgctggtgcgggccggtgcacgtcacccagctgcggtacgccgccgaggacgtcgacctcgccggcacccggatccgcaggggcgacgccgtgcaggccgtcctggtctcggcgaaccacgacccgcgccactacaccgaccccgaacgcctggacctgacccggcagcccgcgggccgcgccgagaaccacgtgggcttcgggcacggggcgcactactgcctgggcgccagcctcgccaggcaggagggtgaggtcgccctgggcgccctgttcgaccgctaccccgacctggcgctggcggtggcgcccgaggagctggagcgcaccccggtgcccggtacctggcggctgacgtcgctgccggtgcgcctgggctga

SEQ ID NO: 32 - protein, 430 aa, Streptomyces lydicus
MSASTSSPLSAHVGKHPGEPHVMDPALISDPFGGYGALREQGPVVRGRFFDDSPLWLVTRFEEVRQVLRDQRFVNNPADPALGVAPEDSPQLRALAMLGIPEHLHGYLLNSILNYDAPDHTRLRRLVSRAFTARKITDLRPRVAQITAELLDRLPEHAEDGVVDLIEHFAYPLPITVICELVGIAAEDRPQWRSWGADLVSVDPDRLGRTFPAMIDHIHALIGQRRAALTDDLLSELIRTHDDDGSRLSDVEMVTLVLTLVLAGHETTAHLIGNGTAALLTHPDQLRLLKDDPALLPRAVHELMRWCGPVHVTQLRYAAEDVDLAGTRIRRGDAVQAVLVSANHDPRHYTDPERLDLTRQPAGRAENHVGFGHGAHYCLGASLARQEGEVALGALFDRYPDLALAVAPEELERTPVPGTWRLTSLPVRLG

SEQ ID NO: 33 - DNA, 1281 bp, Streptomyces tubercidicus
atgaactctccgttcgccgcgcacgtcgggaaacacccgggcgagccgaatgtgatggaccccgccctgatcaccgacccgttcaccggctacggcgcgctgcgtgagcagggcccggtcgtacggggccggttcatggacgactcgcccgtctggctggtgacgcggttcgaggaggtccgccaggtcctgcgcgaccagcggttcgtgaacaatccggcctcgccgtccctgaactacgcgcccgaggacaacccgctgacccggctgatggagatgctgggcctccccgagcacctccgcgtctacctgctcggatcgatcctcaactacgacgcccccgaccacacccggctgcgccgtctggtgtcgcgggcgttcacggcccgcaagatcaccgacctgcggccccgggtcgagcagatcgccgacgcgctgctggcccggctgcccgagcacgccgaggacggcgtcgtcgacctcatccagcacttcgcctaccccctgccgatcaccgtcatctgcgaactggtcggcatacccgaagcggaccgcccgcagtggcgaacgtggggcgccgacctcatctcgatggatccggaccggctcggcgcctcgttcccggcgatgatcgagcacatccatcagatggtccgggaacggcgcgaggcgctcaccgacgacctgctcagcgaactgatccgcacccatgacgacgacggcgggcggctcagcgacgtcgagatggtcaccatgatcctcacgctcgtcctcgccggccacgagaccaccgcccacctcatcagcaacggcacggcggcgctgctcacccaccccgaccagctgcgtctggtcaaggacgatccggccctcctcccccgtgccgtccacgagctgatgcgctggtgcgggccggtgcacatgacccagctgcgctacgccaccgccgacgtcgacctcgccggcacaccgatccgccagggcgatgccgttcaactcatcctggtatcggccaacttcgacccccgtcactacaccgaccccgaccgcctcgatctcacccggcaccccgcgggccacgccgagaaccatgtgggtttcggccatggagcgcactactgcctgggcgccacactcgccaaacaggaaggtgaagtcgccttcggcaaactgctcacgcactacccggacatatcgctgggcatcgccccggaacacctggagcggacaccgctgccgggcaactggcggctgaactcgctgccggtgcggttggggtga

SEQ ID NO: 34 - protein, 426 aa, Streptomyces tubercidicus
MNSPFAAHVGKHPGEPNVMDPALITDPFTGYGALREQGPVVRGRFMDDSPVWLVTRFEEVRQVLRDQRFVNNPASPSLNYAPEDNPLTRLMEMLGLPEHLRVYLLGSILNYDAPDHTRLRRLVSRAFTARKITDLRPRVEQIADALLARLPEHAEDGVVDLIQHFAYPLPITVICELVGIPEADRPQWRTWGADLISMDPDRLGASFPAMIEHIHQMVRERREALTDDLLSELIRTHDDDGGRLSDVEMVTMILTLVLAGHETTAHLISNGTAALLTHPDQLRLVKDDPALLPRAVHELMRWCGPVHMTQLRYATADVDLAGTPIRQGDAVQLILVSANFDPRHYTDPDRLDLTRHPAGHAENHVGFGHGAHYCLGATLAKQEGEVAFGKLLTHYPDISLGIAPEHLERTPLPGNWRLNSLPVRLG

SEQ ID NO: 35 - DNA, 195 bp, Streptomyces tubercidicus
atgcggatcacgatcgacaccgacatctgtatcggcgccggccagtgcgccctgaccgcgccgggagtgttcacccaggacgacgacggcttcagcgccctgctgcccggccgcgaggacggtgcgggcgacccgctggtgcgggaggccgcccgcgcctgcccggtgcaggccatcacggtcacggacgactga

SEQ ID NO: 36 - protein, 64 aa, Streptomyces tubercidicus
MRITIDTDICIGAGQCALTAPGVFTQDDDGFSALLPGREDGAGDPLVREAARACPVQAITVTDD

SEQ ID NO: 37 - DNA, 195 bp, Streptomyces tubercidicus
atgcggatcaccatcgacaccgacatctgcatcggcgccggccagtgcgccctgaccgcgccgggagtcttcacccaggacgacgacggtttcagcgccctgctgcccggccgcgaggacggcgcgggcgacccgctggtgcgcgaggccgcccgcgcctgccccgtgcaggccatttcggtcacggacgactga

SEQ ID NO: 38 - protein, 64 aa, Streptomyces tubercidicus
MRITIDTDICIGAGQCALTAPGVFTQDDDGFSALLPGREDGAGDPLVREAARACPVQAISVTDD

SEQ ID NO: 39 - protein, 9 aa, Artificial Sequence
  Synthetic peptide.
YPYDVPDYA

SEQ ID NO: 40 - protein, 10 aa, Artificial Sequence
  Synthetic peptide.
EQKLISEEDL

SEQ ID NO: 41 - protein, 11 aa, Artificial Sequence
  Synthetic peptide.
QPELAPEDPED

SEQ ID NO: 42 - protein, 11 aa, Artificial Sequence
  Synthetic peptide.
YTDIEMNRLGK

SEQ ID NO: 43 - protein, 7 aa, Streptomyces tubercidicus
IAGHETT

SEQ ID NO: 44 - DNA, 20 bp, Streptomyces tubercidicus
  misc_feature (6)..(18): Nucleotides 6, 9 and 18 are “s” wherein “s” = g or c.
atcgcsggscacgagacsac

SEQ ID NO: 45 - protein, 7 aa, Streptomyces tubercidicus
VAGHETT

SEQ ID NO: 46 - DNA, 20 bp, Streptomyces tubercidicus
  misc_feature (3)..(18): Nucleotides 3, 6, 9, and 18 are “s” wherein “s” = g or c.
gtsgcsggscacgagacsac

SEQ ID NO: 47 - protein, 7 aa, Streptomyces tubercidicus
LAGHETT

SEQ ID NO: 48 - DNA, 20 bp, Streptomyces tubercidicus
  misc_feature (3)..(18): Nucleotides 3, 6, 9, and 18 are “s” wherein “s” = g or c.
ctsgcsggscacgagacsac

SEQ ID NO: 49 - protein, 9 aa, Streptomyces tubercidicus
LLLIAGHET

SEQ ID NO: 50 - DNA, 25 bp, Streptomyces tubercidicus
  misc_feature (2)..(17): Nucleotides 2, 5, 8, 14, and 17 are “s” wherein “s” = g or c.
tsctsctsatcgcsggscacgagac

SEQ ID NO: 51 - protein, 9 aa, Streptomyces tubercidicus
HQCLGQNLA

SEQ ID NO: 52 - DNA, 26 bp, Streptomyces tubercidicus
  misc_feature (12)..(24): Nucleotides 12, 15, and 24 are “s” wherein “s” = g or c.
gtggtcacggasccstgcttggascg

SEQ ID NO: 53 - protein, 8 aa, Streptomyces tubercidicus
FGHGVHQC

SEQ ID NO: 54 - DNA, 24 bp, Streptomyces tubercidicus
  misc_feature (6)..(15): Nucleotides 6, 12, and 15 are “s” wherein “s” = g or c.
aagccsgtgccscasgtggtcacg

SEQ ID NO: 55 - protein, 8 aa, Streptomyces tubercidicus
FGFGVHQC

SEQ ID NO: 56 - DNA, 24 bp, Streptomyces tubercidicus
  misc_feature (6)..(15): Nucleotides 6, 12, and 15 are “s” wherein “s” = g or c.
aaggcsaagccscasgtggtcacg

SEQ ID NO: 57 - protein, 8 aa, Streptomyces tubercidicus
FGHGIHQC

SEQ ID NO: 58 - DNA, 24 bp, Streptomyces tubercidicus
  misc_feature (6)..(12): Nucleotides 6 and 12 are “s” wherein “s” = g or c.
aagccsgtgccstaggtggtcacg

SEQ ID NO: 59 - protein, 8 aa, Streptomyces tubercidicus
FGHGVHFC

SEQ ID NO: 60 - DNA, 24 bp, Streptomyces tubercidicus
  misc_feature (6)..(15): Nucleotides 6, 12, and 15 are “s” wherein “s” = g or c.
aagccsgtgccscasgtgaagacg

SEQ ID NO: 61 - protein, 24 aa, Streptomyces tubercidicus
HPGEPNVMDPALITDPFTGYGALR

SEQ ID NO: 62 - protein, 21 aa, Streptomyces tubercidicus
FVNNPASPSLNYAPEDNPLTR

SEQ ID NO: 63 - protein, 19 aa, Streptomyces tubercidicus
LLTHYPDISLGIAPEHLER

SEQ ID NO: 64 - protein, 17 aa, Streptomyces tubercidicus
VYLLGSILNYDAPDHTR

SEQ ID NO: 65 - protein, 13 aa, Streptomyces tubercidicus
TWGADLISMDPDR

SEQ ID NO: 66 - protein, 13 aa, Streptomyces tubercidicus
EALTDDLLSELIR

SEQ ID NO: 67 - protein, 12 aa, Streptomyces tubercidicus
FMDDSPVWLVTR

SEQ ID NO: 68 - protein, 12 aa, Streptomyces tubercidicus
LMEMLGLPEHLR

SEQ ID NO: 69 - protein, 11 aa, Streptomyces tubercidicus
VEQIADALLAR

SEQ ID NO: 70 - protein, 11 aa, Streptomyces tubercidicus
LVKDDPALLPR

SEQ ID NO: 71 - protein, 8 aa, Streptomyces tubercidicus
DDPALLPR

SEQ ID NO: 72 - protein, 8 aa, Streptomyces tubercidicus
TPLPGNWR

SEQ ID NO: 73 - protein, 7 aa, Streptomyces tubercidicus
LNSLPVR

SEQ ID NO: 74 - protein, 7 aa, Streptomyces tubercidicus
ITDLRPR

SEQ ID NO: 75 - protein, 7 aa, Streptomyces tubercidicus
EQGPVVR

SEQ ID NO: 76 - protein, 7 aa, Streptomyces tubercidicus
AVHELMR

SEQ ID NO: 77 - protein, 5 aa, Streptomyces tubercidicus
AFTAR

SEQ ID NO: 78 - protein, 5 aa, Streptomyces tubercidicus
FEEVR

SEQ ID NO: 79 - protein, 7 aa, Streptomyces tubercidicus
PGEDNVM

SEQ ID NO: 80 - DNA, 21 bp, Streptomyces tubercidicus
  misc_feature (3)..(18): Nucleotides 3, 6, 12, and 18 are “s” wherein “s” = c or g.
ccsggsgarccsaaygtsatg

SEQ ID NO: 81 - protein, 7 aa, Streptomyces tubercidicus
ALITDPF

SEQ ID NO: 82 - DNA, 21 bp, Streptomyces tubercidicus
  misc_feature (3)..(18): Nucleotides 3, 6, 12, and 18 are “s”wherein “s” = c or g.
gcsctsatyacsgacccsttc

SEQ ID NO: 83 - protein, 8 aa, Streptomyces tubercidicus
FMDDSPVW

SEQ ID NO: 84 - DNA, 24 bp, Streptomyces tubercidicus
  misc_feature (13)..(13): Nucleotide 13 is “w” wherein “w” = a or t.
ttcatggacgacwssccsgtstgg

SEQ ID NO: 85 - protein, 8 aa, Streptomyces tubercidicus
LNYDAPDH

SEQ ID NO: 86 - DNA, 24 bp, Streptomyces tubercidicus
  misc_feature (3)..(18): Nucleotides 3, 15 and 18 are “s” wherein “s” = c or g.
ctsaaytaygacgcsccsgaccac

SEQ ID NO: 87 - protein, 8 aa, Streptomyces tubercidicus
VEQIADAL

SEQ ID NO: 88 - DNA, 24 bp, Streptomyces tubercidicus
  misc_feature (3)..(24): Nucleotides 3, 15, 21, and 24 are “s” wherein “s” = c or g.
gtsgarcagatygcsgacgcscts

SEQ ID NO: 89 - protein, 8 aa, Streptomyces tubercidicus
DLISMDPD

SEQ ID NO: 90 - DNA, 24 bp, Streptomyces tubercidicus
  misc_feature (6)..(21): Nucleotides 6, 11, 12, and 21 are “s” wherein “s” = c or g.
ctggastarwsstacctgggsctg

SEQ ID NO: 91 - DNA, 36 bp, Streptomyces tubercidicus
agattaattaatgtcggaattaatgaactgtccgtt

SEQ ID NO: 92 - DNA, 32 bp, Streptomyces tubercidicus
aaactcaccccaaccgcaccggcagcgagttc

SEQ ID NO: 93 - protein, 7 aa, Streptomyces tubercidicus
MSELMNS

SEQ ID NO: 94 - DNA, 1293 bp, Streptomyces
atgtcggcaatatccagctccccgttcgccgcacacgtcggaaagcatcccggcgagccgaatgtgatggacccggcgctgatcaccgacccgttcggcggctacggcgcactgcgtgagcaaggccccgtcctaccgggccggttcatggacgactcacccgtctggctcgtgacgcgcttcgaagaggtccgccaagtcctgcgcgatcagcggttcctgaacaacccggccgcgtcgtcaccggggcattcgatcgacgagagccccacggccaggctgctggacatgatggggatgcccgaacatttccggccgtatctgatggggtcgatcctcaacaacgacgcccccgaccacacccggctgcgccgtctggtgtcacgcgcgttcacggcacgcaagatcaccgatctgcggccgcgggtcgagcagctcgccgacgagctgctggcccggcttcccgagcacgccgaggacggtgtggtcgacctgatcaagcacttcgcctatcccctgccgatcaccgtgatctgcgaactggtcggcatcccggaagcggaccgcccgcaatggcggaagtggggcgccgacctcgtttcgctgcagccggagcggctcagcacctcgttcccggcgatgatcgagcacatccatgaactgatccgcgagcggcgcggcgcgctcaccgacgatctgctcagcgagctgatccgtacccatgacgacgacggcagccggctcagcgacgtcgagatggtcaccatggtcctcaccgtcgtcctggccggccacgagaccaccgcccacctgataggcaacggcacggcggcgctgctcacccaccccgaccagctgcgcctggtcaaggacgacccggagctgcttccgcgtgccgtccacgagctgctgcgctggtgcgggccggtccagatgacccagctgcggtacgcctccgaggatgtcgagatcgccgggacgccgatccgtaagggcgacgccgtacaactcatcctggtatcggcgaacttcgacccccgccactacaccgcccccgaacgcctcgacctgacccgccaccccgccggccacgccgagaaccatgtgggcttcggccacggaatgcactactgcctgggcgccaccctcgccaaacaggagggcgaagtcgcgttcggcaagctcttcacgcactacccggagctgtcgctggccgtcgcaccggacgagttggagcgaacgccggtgcccggcagctggcggttggattcgctgccggtgcggttggggtga

SEQ ID NO: 95 - protein, 430 aa, Streptomyces
MSAISSSPFAAHVGKHPGEPNVMDPALITDPFGGYGALREQGPVLPGRFMDDSPVWLVTRFEEVRQVLRDQRFLNNPAASSPGHSIDESPTARLLDMMGMPEHFRPYLMGSILNNDAPDHTRLRRLVSRAFTARKITDLRPRVEQLADELLARLPEHAEDGVVDLIKHFAYPLPITVICELVGIPEADRPQWRKWGADLVSLQPERLSTSFPAMIEHIHELIRERRGALTDDLLSELIRTHDDDGSRLSDVEMVTMVLTVVLAGHETTAHLIGNGTAALLTHPDQLRLVKDDPELLPRAVHELLRWCGPVQMTQLRYASEDVEIAGTPIRKGDAVQLILVSANFDPRHYTAPERLDLTRHPAGHAENHVGFGHGMHYCLGATLAKQEGEVAFGKLFTHYPELSLAVAPDELERTPVPGSWRLDSLPVRLG

SEQ ID NO: 96 - DNA, 18 bp, Streptomyces
cgsccsccsctswssaas

SEQ ID NO: 97 - DNA, 21 bp, Streptomyces
sassgcsttsbcccartgytc

SEQ ID NO: 98 - DNA, 1266 bp, Streptomyces
gtggtcgacgcacaccagacgttcgtcatcgtcgggggtggcctggccggcgcaaaggccgcggagactctccgcgcggaggggttcaccggccgggtgatcctcatctgtgacgagcgcgaccacccgtacgagcgccccccgctctccaaggggttcctgctcggcaaggaagagcgcgacagcgtgttcgtccatgagcccgcctggtacgcccaggcacagatcgaactgcacctgggccagcccgccgtccgcctcgaccccgagggcaggaccgtccgcctcggcgacggcaccctgatcgcctacgacaagctgctgctggccaccggcgccgaaccgcggcgcctggacatccccggcaccggcctggccggcgtgcaccacctgcgccgcctcgcccacgccgaacggctgcgcggcgtcctggcctccctcggccgcgacaacggccatctggtgatcgccggagccggctggatcggcctggaggtcgccgccgcggcccgctcctacggcgccgaggtgaccgtcgtcgaggccgccccgacgccgctgcacggcatcctggggcccgaactcggcggtctgttcaccgatctgcaccgcgagcacggcgtccgcttccacttcggcgcccgcttcaccgagatcgtcggagagggcggcatggtgctcgccgtgcgcaccgacgacggcgaggaacaccccgcccacgatgtgctcgccgcgatcggcgccgccccgcgcaccgcgctcgccgaacaggccgggctggatctcgccgacccggagaccggcggcggggtggccgtcgacgcggcgctgcgcacctccgacccgtacatctacgccgccggtgacgtcgccgccgccgaccacccgctgctggacacccggctgcgggtcgaacactgggccaacgccctcaacggcggcccggccgccgcccgcgccatgctcggccaggacatcagctacgaccgcatcccgtacttcttctccgaccagtacgacgtcggcatggagtactccggctacgccccgcccggctcgtacgcccaggtcgtctgccgcggcgacgtcgccaagcgggagttcatcgccttctggctggcggcggacggccggctgctcgcgggcatgaacgtcaacgtctgggacgtcgccgagtccatccagcaactcatccgctccggggcgccgttggagcccggcgcactggccgatccgcaggttccgctggcggcactgctcccgtag

SEQ ID NO: 99 - protein, 421 aa, Streptomyces
VVDAHQTFVIVGGGLAGAKAAETLRAEGFTGRVILICDERDHPYERPPLSKGFLLGKEERDSVFVHEPAWYAQAQIELHLGQPAVRLDPEGRTVRLGDGTLIAYDKLLLATGAEPRRLDIPGTGLAGVHHLRRLAHAERLRGVLASLGRDNGHLVIAGAGWIGLEVAAAARSYGAEVTVVEAAPTPLHGILGPELGGLFTDLHREHGVRFHFGARFTEIVGEGGMVLAVRTDDGEEHPAHDVLAAIGAAPRTALAEQAGLDLADPETGGGVAVDAALRTSDPYIYAAGDVAAADHPLLDTRLRVEHWANALNGGPAAARAMLGQDISYDRIPYFFSDQYDVGMEYSGYAPPGSYAQVVCRGDVAKREFIAFWLAADGRLLAGMNVNVWDVAESIQQLIRSGAPLEPGALADPQVPLAALLP

SEQ ID NO: 100 - DNA, 1314 bp, Streptomyces
atgcccgctgcacgccgccgccttcgacctccgcaccggagcggcgacctgcctgcccgcccgccgggccgtgcgcacccaccccgtgaccgtccaggacggcatgatctacgtccatcacgccgcggaggagggcaccgccgcatgaagtcggtcgctgtcatcggggcctcgctggcgggcctgtacgccgcgcggtccctgcgttcccaggggttcgacggccgcctggtgatcgtcggggacgagtgccacggcccctacgaccggcccccgctgtccaaggacttcctcaccggcgccaccgacccgggccgactcgccctggccgacgccgaggagatcgccgaactcgacgccgaatggctgctgggcacccgggccaccgggctcgacaccggcggacgcacggtgctgctcgatggcggccggtccctgaccaccgacggcgtggtcctcgccaccggcgccgccccgcgcctgctccccggaccggtgcccgccggggtccacaccctgcgcaccctcgacgacgcccaggcgctccgtgcggatctggcgccggcgccggtccgggtcgtggtgatcggcggcggcttcatcggcgccgaggtcgcctcgtcctgcgccgccctaggccatgacgtcaccgtggtcgaggccgcgccgctccccctcgtcccccaactcggccacgccatggccgagatctgcgccgccctgcatgcggaccacggcgtcacgctgctcaccggaaccggtgtcgcccggctgcgcagcgagggcgacggccggcgcgtcaccggcgtcgagctgaccgacggccgcctgctccccgccgacgtggtcgtcgtcggcatcggggtacgcccccgcaccgcctggctcacggactccggactgccgctcgacgacggtgtgctctgcgacgcgggctgtgtcaccccgctgcccgccgtcgtggccgtcggcgacgtcgccagggtggacggcgcccgtgccgagcactggaccagcgccaccgaacaggccgccgtggcggcgcggaacctgctggccggcagcaccgtcgcgacccaccggagcctgccgtacttctggtccgaccagtacggcgtccgcatccagttcgcgggccaccggctgcccaccgacacaccgcgcgtcctcgaaggctcccccgacgaccgcagcttcctcgcctgttacgaacgggacggacgcaccaccgcggtgctcgccctcaaccggccccgccccttcatgcggctccgccgcgaactcgcccgcaccgccctgtcggccaccacctga

SEQ ID NO: 101 - protein, 437 aa, Streptomyces
MPAARRRLRPPHRSGDLPARPPGRAHPPRDRPGRHDLRPSRRGGGHRRMKSVAVIGASLAGLYAARSLRSQGFDGRLVIVGDECHGPYDRPPLSKDFLTGATDPGRLALADAEEIAELDAEWLLGTRATGLDTGGRTVLLDGGRSLTTDGVVLATGAAPRLLPGPVPAGVHTLRTLDDAQALRADLAPAPVRVVVIGGGFIGAEVASSCAALGHDVTVVEAAPLPLVPQLGHAMAEICAALHADHGVTLLTGTGVARLRSEGDGRRVTGVELTDGRLLPADVVVVGIGVRPRTAWLTDSGLPLDDGVLCDAGCVTPLPAVVAVGDVARVDGARAEHWTSATEQAAVAARNLLAGSTVATHRSLPYFWSDQYGVRIQFAGHRLPTDTPRVLEGSPDDRSFLACYERDGRTTAVLALNRPRPFMRLRRELARTALSATT

SEQ ID NO: 102 - DNA, 1233 bp, Streptomyces
atggcccagaacacggcattcatcatcgcgggagcggggctggccggggcgaaggccgcggagacactgcgcgcggagggcttcggcggccccgtcctgctgctgggcgacgagcgcgagcgtccctacgagcggccgccgctgtccaagggctacctcttgggcacctccgagcgggagaaggcgtacgtccatccgccccagtggtacgccgagcacgacgtcgatctgcggctgggcaacgccgtcaccgccctcgacccggccggccacgaggtgaccctcgccgacggcagccggctgggctacgccaagctgctgctggccaccggctccactccgcgccggctgccggtgcccggcgccgacctcgacggggtccacacgctgcggtacctggcggacagcgaccgcctcaaggacctcttccggtccgcgtcccggatcgtggtgatcggcggcggctggatcggcctggagaccacggccgccgcgcgtgcggcgggggtcgaggtgaccgtgctggagtcggcgccgctgcccctgctgggggtgctgggccgcgaggtcgcccaggtcttcgccgatctgcacaccgagcacggtgtcgcgctgcgctgcgacacccaggtcacggagatcaccggcacgaacggcgcggtcgacggggtacggctggccgacggcacccggatcgcggccgacgcggtgatcgtcggcgtcgggatcacccccaactccgagacggccgccgcggccgggctcaaggtcgacaacggcgtcgtcgtggacgagcggctgtgctcctcccacccggacatctacgccgccggcgacgtcgccaacgcctaccaccccctcctgggcaagcacctccgcgtcgagcactgggccaacgccctccaccagccgaagaccgcggcccgggccatgctgggcggggaggccggctacgaccggctgccgtacttcttcaccgaccagtacgacctgggcatggagtacacggggcatgtggagccgggcgggtacgaccgcgtggtgttccgcggcgacaccggtgcccgcgagttcatcgccttctggctctccggcggccgggtgctggccgggatgaatgtgaacgtatgggacgtcaccgacccgatccgggccctggtggcgagcgggcgggccgtggaccccgagcggctcgccgacgcggacgtaccgctggcggatctggtcccctga

SEQ ID NO: 103 - protein, 410 aa, Streptomyces
MAQNTAFIIAGAGLAGAKAAETLRAEGFGGPVLLLGDERERPYERPPLSKGYLLGTSEREKAYVHPPQWYAEHDVDLRLGNAVTALDPAGHEVTLADGSRLGYAKLLLATGSTPRRLPVPGADLDGVHTLRYLADSDRLKDLFRSASRIVVIGGGWIGLETTAAARAAGVEVTVLESAPLPLLGVLGREVAQVFADLHTEHGVALRCDTQVTEITGTNGAVDGVRLADGTRIAADAVIVGVGITPNSETAAAAGLKVDNGVVVDERLCSSHPDIYAAGDVANAYHPLLGKHLRVEHWANALHQPKTAARAMLGGEAGYDRLPYFFTDQYDLGMEYTGHVEPGGYDRVVFRGDTGAREFIAFWLSGGRVLAGMNVNVWDVTDPIRALVASGRAVDPERLADADVPLADLVP

SEQ ID NO: 104 - DNA, 1266 bp, Streptomyces
gtggtcgacgcacaccagacgttcgtcatcgtcgggggtggcctggccggcgcaaaggccgcggagactctccgcgcggaagggttcaccggccgggtgatcctcatctgtgacgagcgcgaccacccgtacgagcgccccccgctctccaaggggttcctgctcggcaaggaagagcgcgacagcgttttcgtccacgaacccgcctggtacgcccaggcacagatcgaactgcacctgggccagcccgccgtccgcctcgaccccgaggcgaagaccgtccgcctcggcgacggcaccctgatcgcctacgacaagctgctgctggccaccggcgccgagccgcgccgcctggacatccccggcaccggcctggccggcgtgcaccacctgcgccgcctcgcccacgccgaacggctgcgcggcgtcctggcctccctcgggcgggacaacgggcatctggtgatcgccggcgccggctggatcggcctggaggtcgccgccgcggcccgctcctacggcgccgaggtcaccgtcgtcgaggccgccccgacaccgctgcacggcatcctggggcccgaactcggcggcctgttcaccgaactgcaccgcgcacacggcgtgcgcttccacttcggcgcccgtttcaccgagatcgtcggacaggacggcatggtgctcgccgtgcgcaccgacgacggcgaggagcaccccgcccacgacgtgctcgccgcgatcggcgccgccccgcgcaccgcactcgccgaacaggccggactcgacctcgccgacccggaggccggcggcggcgtggccgtcgacgcgacgctgcgcacctccgacccgtacatctacgccgccggcgacgtggccgccgccgaccaccccctcctggacacccggctgcgcgtcgaacactgggccaacgccctcaacggcggcccggccgccgcgcgcgccatgctcggccaggacatcagctacgaccgcgtcccgtacttcttctccgaccagtacgacgtcggcatggagtactccggctacgccccgcccggctcctacgcacaggtcgtctgccgcggcgacgtcgccaaacgggagttcatcgcgttctggctcggcgaggacggacggctgctcgcggggatgaacgtcaacgtctgggacgtcgccgaaaccatccagcaactcatccgcggcggggtgcggttggagcccggcgagctggctgatccggaggttccgctgacctcactgctcccgtag

SEQ ID NO: 105 - protein, 421 aa, Streptomyces
VVDAHQTFVIVGGGLAGAKAAETLRAEGFTGRVILICDERDHPYERPPLSKGFLLGKEERDSVFVHEPAWYAQAQIELHLGQPAVRLDPEAKTVRLGDGTLIAYDKLLLATGAEPRRLDIPGTGLAGVHHLRRLAHAERLRGVLASLGRDNGHLVIAGAGWIGLEVAAAARSYGAEVTVVEAAPTPLHGILGPELGGLFTELHRAHGVRFHFGARFTEIVGQDGMVLAVRTDDGEEHPAHDVLAAIGAAPRTALAEQAGLDLADPEAGGGVAVDATLRTSDPYIYAAGDVAAADHPLLDTRLRVEHWANALNGGPAAARAMLGQDISYDRVPYFFSDQYDVGMEYSGYAPPGSYAQVVCRGDVAKREFIAFWLGEDGRLLAGMNVNVWDVAETIQQLIRGGVRLEPGELADPEVPLTSLLP